SEQUENCE LISTING (10 sequences)

SEQ ID NO: 1 - protein, 28 aa, Homo sapiens
DFGISAQIGATLARRLSFIGTPYWMAPE

SEQ ID NO: 2 - DNA, 24 bp, Artificial Sequence
  Description of Artificial Sequence Synthetic primer
gggagccaagaaatttgagagctc

SEQ ID NO: 3 - DNA, 21 bp, Artificial Sequence
  Description of Artificial Sequence Synthetic primer
ccggtggatgtggaatgtgtg

SEQ ID NO: 4 - DNA, 21 bp, Artificial Sequence
  Description of Artificial Sequence Synthetic primer
cccttctgtctcctccaccac

SEQ ID NO: 5 - DNA, 64 bp, Artificial Sequence
  Description of Artificial Sequence Synthetic nucleotide construct
gatcccctagagacccccgggaccacttcaagagagtggtcccgggggtctctatttttggaaa

SEQ ID NO: 6 - DNA, 64 bp, Artificial Sequence
  Description of Artificial Sequence Synthetic nucleotide construct
gatccccttctgtggggctggttctcttcaagagagagaaccagccccacagaatttttggaaa

SEQ ID NO: 7 - protein, 28 aa, Homo sapiens
DFGVSGELTASVAKRRSFIGTPYWMAPE

SEQ ID NO: 8 - protein, 28 aa, Homo sapiens
DFGVAAKITATIAKRKSFIGTPYWMAPE

SEQ ID NO: 9 - protein, 28 aa, Homo sapiens
DFGVSAQITATIAKRKSFIGTPYWMAPE

SEQ ID NO: 10 - protein, 28 aa, Homo sapiens
DFGFCAQINELNLKRTTMVGTPYWMAPE

(19) United States
(12) Patent Application Publication
(10) Pub. No.: US 2007/0087988 A1
(43) Pub. Date: Apr. 19, 2007

(54) Hematopoietic progenitor kinase 1 for modulation of an immune response

(75) Inventors: Sansana Sawasdikosol, Jersey City, NJ (US); Saba Alzabin, New York, NY (US); Steven Burakoff, New York, NY (US)

Correspondence Address: WILMER CUTLER PICKERING HALE AND DORR LLP, 60 STATE STREET, BOSTON, MA, 02109, US

(73) Assignee: New York University, New York City, NY (US)

(21) Appl. No.: 11/542,031

(22) Filed: Oct. 2, 2006

Related U.S. Application Data
(60) Provisional application No. 60/722,133, filed on Sep. 30, 2005.

(51) Int. Cl. A61K 48/00; C12Q 1/68

(52) U.S. Cl. 514/44; 435/6

(57) ABSTRACT

Methods of regulating hematopoietic progenitor kinase 1 (HPK1) are described as are methods of identifying compounds that can regulate HPK1.

### FEDERALLY SPONSORED RESEARCH OR DEVELOPMENT

The U.S. Government may have certain rights in this invention pursuant to Grant No. 5R01 CA070758-06 awarded by National Cancer Institute.

### TECHNICAL FIELD

This application relates to the field of immunology, and more particularly to T cell receptor-mediated pathways.

### BACKGROUND

Hematopoietic progenitor kinase 1 (HPK1) is a hematopoietic cell-restricted Ste20 serine/threonine kinase. HPK1 kinase activity can be induced by activation signals generated by various different cell surface receptors found in hematopoietic cells upon ligand engagement. Ligand engagement or antibody-mediated crosslinking of T cell receptors (TCR), B cell antigen receptor (BCR) (Liou et al., 2000, Immunity 12:399), transforming growth factor β receptor (TGF-βR) (Wang et al., 1997. J. Biol. Chem. 272:22771; Zhou et al., 1999, J. Biol. Chem. 274:13133), erythropoietin receptor (EPOR) (Nagata et al., 1999, Blood 93:3347), and Fas (Chen et al., 1999, Oncogene 18:7370) can induce HPK1 kinase activity. Each receptor utilizes unique, but sometimes overlapping, signaling mechanisms to activate HPK1. HPK1 acts as a down-modulator of T and B cell functions through the AP-1, NFκB, Erk2, and Fos pathways, and for example, HPK1 has been implicated as a negative regulator of signal transduction in T and B cells.

Lipopolysaccharide (LPS) is a component of the gram-negative bacterial cell wall. It is a potent maturation stimulus that activates dendritic cells upon binding to toll-like receptors (TLRs) 2 and 4 as well as to CD14 (Tsan et al., 2004, Am. J. Physiol. Cell Physiol. 286:C739-C744). In macrophages, LPS has been shown to activate the cyclooxygenase-2 (COX-2) pathway, which leads to prostaglandin production (Metzger et al., 1981, J. Immunol. 127:1109-1113; Lee et al., 1992, J. Biol. Chem. 267:25934). Prostaglandin E2 produced by activated macrophages creates a negative feedback loop by acting as an inhibitor of early and late processes involved in macrophage activation (Inoue et al., 2000, J. Biol. Chem. 275:28028-28032). This happens through the binding of PGE₂ to two of its four G-protein-coupled receptors, the E-prostanoid receptor 2 (EP2) and 4 (EP4), which are known to be inducers of cyclic adenosine monophosphate (cAMP) production (Ikegami et al., 2001, J. Immunol. 166:4689-4696. Recently it has been shown that PGE₂ also regulates dendritic cell functions through EP2 and EP4 receptors (Harizi et al., 2003, J. Leuk. Biol. 73:756-763).

### SUMMARY

The invention relates to the biological cascade that regulates HPK1 activity, identification of compounds that modulate HPK1, and methods of modulating immune system activity by modulation of HPK1.

Accordingly, in one aspect, the invention relates to a method of identifying a candidate compound for modulating hematopoietic progenitor kinase 1 (HPK1) activity. The method includes contacting an HPK1 or a fragment thereof that includes ser171 of HPK1 with a test compound; and determining whether the test compound binds to the HPK1 or fragment thereof at a site that modulates protein kinase A (PKA) binding or phosphorylation of the ser171, such that a compound that binds to the HPK1 or fragment thereof at a site that modulates the PKA binding or phosphorylation of the ser171 is a candidate compound for modulating HPK1 activity. In some embodiments the test compound decreases PKA binding or phosphorylation of the ser171 and is a candidate compound for inhibiting HPK1 activity. In another embodiment, the compound increases PKA binding or phosphorylation of the ser171 and is a candidate compound for enhancing HPK1 activity. In some cases, the compound inhibits HPK1 activation and enhances dendritic cell maturation or migration or the compound increases HPK1 activation and decreases dendritic cell maturation or migration. The compound is, in some embodiments, a cell-permeable peptide, a pseudo substrate for HPK1, or a competitive inhibitor of ATP binding to a PKA kinase domain.

Another aspect of the invention relates to a method of identifying a candidate compound for enhancing dendritic cell (dendritic cell) maturation or migration. The method includes contacting a cell with a test compound; and determining whether the test compound decreases HPK1 expression or activity compared to a control cell that is not contacted with the test compound, such that a test compound that decreases HPK1 expression or activity is a candidate compound for enhancing dendritic cell maturation or migration. In certain embodiments of the invention, dendritic cell maturation is induced by lipopolysaccharide (LPS). Other embodiments include determining whether the test compound increases dendritic cell maturation or migration. In another embodiment, increased dendritic cell maturation is indicated by increased expression or increased activity of at least one of CD80 (B7.1), CD86 (B7.2), CD83, MHC class II, or CCR7.

An aspect of the invention also includes a method of enhancing maturation or migration of dendritic cells. The method includes contacting a cell with a compound that decreases the level of HPK1 expression or HPK1 activity in a dendritic cell compared to a dendritic cell that is not contacted with the compound, such that the decrease in the HPK1 expression or activity is indicative of increased maturation or migration of dendritic cells. In the method, the compound contacted cell is a dendritic cell. In some embodiments, dendritic cell maturation is induced by lipopolysaccharide (LPS). In another embodiment, the compound is an siRNA. The expression or activity of CD80 (B7.1), CD86 (B7.2), CD83, MHC class II, or CCR7 is increased by contact with the compound. In some embodiments, the dendritic cells are in a human. In other embodiments, the cells are in a non-human subject.

Another aspect of the invention relates to a method of identifying a candidate compound for modulating HPK1 activity. The method includes contacting a cell that expresses HPK1 with a test compound; and determining whether the test compound modulates Casita lineage lymphoma-b (Cbl-b) expression or activity in the contacted cell, such that the ability of the test compound to modulate Cbl-1 is indicative of the ability of the compound to modulate HPK1 activity. The method can also include determining whether the test compound can modulate HPK1 activity. In some embodiments of the invention, the test compound decreases Cbl-b expression or activity and decreases HPK1 activity in the cell, and in certain embodiments, the ubiquitin ligase activity of Cbl-b is not decreased by the test compound in the contacted cell. The test compound can be an siRNA.

The invention is, in another aspect, a method of identifying a candidate compound for modulating an immune response. The method includes contacting a cell that expresses HPK1 with a test compound; determining whether the test compound modulates Cbl-b expression or activity in the contacted cell; and determining whether the test compound modulates HPK1 activity in the contacted cell, such that the test compound that modulates Cbl-1 and HPK1 activity is a candidate compound for modulating an immune response. In certain embodiments of the invention, the test compound decreases Cbl-1 expression or activity and decreases HPK1 activity in the contacted cell. In some embodiments, the test compound does not decrease the ubiquitin ligase activity of Cbl-1 in the contacted cell.

In another aspect, the invention relates to a method of identifying a candidate compound for modulating a T cell response in an animal, e.g., via modulation of HPK1 activity, in which the method includes administering a test compound to an animal, the compound having the ability to decrease Cbl-b expression or activity in a cell that expresses HPK1; and determining whether the animal has an increased T cell response to T cell receptor (TCR) stimulation compared to a wild type animal to which the test compound was not administered, such that an increased or decreased T cell response in the animal is indicative of the ability of the test compound to modulate T cell activity and the test compound is a candidate compound for modulating HPK1 activity. In some embodiments of the invention, the increased response to TCR stimulation is at least one of splenomegaly, hyperproliferation of at least one type of hematopoietic cell, resistance to PGE₂-induced immune suppression, or augmented dendritic cell function. Augmented dendritic cell function can be assayed by, for example, at least one of expression of at least one maturation marker, priming/activation of T cells, migration of cells to regional lymph nodes, secretion of IL-12, secretion of IL-6, or secretion of TNF-alpha

In some aspects, the invention relates to a method of identifying a compound that enhances an immune response. The method includes administering a compound to a hematopoietic cell; and assaying the hematopoietic cell for at least one indication of HPK1 inhibition, such that HPK1 inhibition is indicative of an enhanced immune response. The indication of HPK1 inhibition is, in certain embodiments, at least one of splenomegaly, hyperproliferation of at least one type of hematopoietic cell, resistance to PGE₂-induced immune suppression, or augmented dendritic cell function. The hematopoietic cell can be in an animal, e.g., a human or a non-human mammal such as a mouse, rat, dog, cat, cow, goat, sheep, guinea pig, or non-human primate. In certain embodiments, augmented dendritic cell function is assayed by comparing dendritic cells from an animal that was administered the compound, with dendritic cells from an animal that was not administered the compound, and the augmented dendritic cell function is indicated by increased expression of at least one maturation marker, e.g., increased expression of CD80 (B7.1), CD86 (B7.2), CD83, MHC class II, CCR7, CD1a, CD1b, CD1c, CD1d, CD40, DC-LAMP, or DC-SIGN. In certain embodiments of the invention, the maturation marker is assayed using a Western blot, Northern blot, real time PCR, or FACS. In other embodiments, HPK1 inhibition is indicated by at least one of enhanced migration of cultured dendritic cells toward a CCL-21/CCL-19 gradient, increased proliferation of T cells, increased stimulation T cells, increased migration of dendritic cells in vivo, or increased cytokine production by T cells. Embodiments of the invention include those in which the enhanced immune response is increased T cell proliferation in the presence of an activator of TCR, concanavalin A (ConA), or other T cell mitogen. In some cases, the enhanced immune response is assayed in T cells that are restimulated with an activator or TCR or ConA T cell mitogen. In yet another embodiment, the level PGE₂-induced immune suppression is determined by assaying IL-2 production in the presence of physiological concentration of PGE₂, and sustained release of IL-2 in the presence of PGE₂ indicates HPK1 inhibition.

In another aspect, the invention relates to a method of modulating an immune response, the method comprising contacting an immune system cell with a compound that modulates HPK1 expression or activity. In certain embodiments of the invention, the compound decreases HPK1 activity and increases dendritic cell maturation relative to a dendritic cell that was not contacted with the compound. In other embodiments, the compound increases HPK1 activity and decreases dendritic cell maturation is relative to a dendritic cell that was not contacted with the compound. In yet another embodiment of the invention, the compound decreases HPK1 activity in the presence of a dendritic cell maturation factor relative to a dendritic cell that was not contacted with the compound. The dendritic cell maturation factor is, in some cases, a lipopolysaccharide (LPS). The immune system cell is, in some embodiments of the invention, a dendritic cell, T cell, a B cell, a monocyte/macrophage, neutrophil, polymorph, natural killer cell, natural killer T cell, eosinophil, granulocyte, erythrocyte, or mast cell. In some cases the subject is, e.g. a human or the subject is a non-human mammal such as a mouse, rat, dog, cat, cow, goat, sheep, guinea pig, or non-human primate. In certain embodiments of the invention, the compound is an siRNA.

Another aspect of the invention is a method of identifying a subject at risk for or having an immune disorder or cancer, e.g., an immune system cancer (antigenic tumor/cancer). The invention includes determining the level of HPK1 activity in a cell obtained from the subject; and comparing the level of HPK1 activity in the cell to a reference level of HPK1, such that a decreased level of HPK1 compared to the reference indicates that the subject is at risk for or has an immune or autoimmune disorder. In certain embodiments, the cell is a hematopoietic cell. The subject can be, e.g. a human or a non-human mammal such as a mouse, rat, dog, cat, cow, goat, sheep, guinea pig, or non-human primate. In some embodiments, the disorders include adult leukemia lymphoma (ALL), chronic myelogenous leukemia, Hodgkin's disease, Hodgkin's lymphoma, plasmacytoma, multiple sclerosis, rheumatoid arthritis, diabetes (type I), and lupus.

Another aspect of the invention is method of identifying a subject at risk for or having a cancer. The method includes determining the level of HPK1 activity in a cell obtained from the subject; and comparing the level of HPK1 activity in the cell to a reference level of HPK1, such that a decreased level of HPK1 compared to the reference indicates that the subject is at risk for or has a cancer. In some embodiments, the cancer is lung cancer, breast cancer, prostate cancer, testicular cancer, a head or neck carcinoma, liver cancer, or bladder cancer. In certain embodiments of the invention, the cell is a hematopoietic cell. In some embodiments, the subject is a human or a non-human mammal such as a mouse, rat, dog, cat, cow, goat, sheep, guinea pig, or non-human primate.

The invention, in another aspect, includes a method of treating a subject at risk for or having an immune disorder. The method comprises providing to a subject in need thereof a pharmaceutically effective amount of a compound that inhibits HPK1 expression or activity, thereby treating or preventing the cancer. In some cases, the compound is provided in a pharmaceutically acceptable excipient. In certain embodiments, the subject is a human or a non-human mammal such as a mouse, rat, dog, cat, cow, goat, sheep, guinea pig, or non-human primate.

In another aspect, the invention relates to a method of treating a subject at risk for or having a cancer. The method includes providing to a subject in need thereof a pharmaceutically effective amount of a compound that inhibits HPK1 expression or activity, thereby treating or preventing the cancer. In one embodiment, the compound is provided in a pharmaceutically acceptable excipient. The subject can be a mammal, e.g., a human or a non-human mammal such as a mouse, rat, dog, cat, cow, pig, goat, or sheep.

In one aspect, the invention relates to a method of altering at least one HPK1-mediated effect, the effect includes increasing IL-2 production, increasing TNF secretion, increasing IFN-γ production increasing T cell proliferation, increasing B cell proliferation decreasing synthesis of an immunosuppressive cytokine, or decreasing apoptosis of T cells, decreasing tumor-induced apoptosis of hematopoetic cells. The method includes providing a cell or organism that can express IL-2, TNF, IFN-γ, or an immunosuppressive cytokine, or providing a T cell that can proliferate, a B cell that can proliferate, or a tumor cell; and contacting the cell or organism with a compound that inhibits HPK1 expression or activity in an amount and for a time sufficient to inhibit HPK1 expression or activity compared to a reference, thereby altering at least one HPK1-mediated effect, the effect comprising increasing IL-2 production, increasing TNF secretion, increasing IFN-γ production increasing T cell proliferation, increasing B cell proliferation, decreasing synthesis of an immunosuppressive cytokine, or inducing apoptosis of a tumor cell. In some embodiments, the cell or organism is a non-small lung cancer cell or an organism having a non-small lung cancer.

In another aspect, the invention relates to a method of specifically altering PGE₂ modulation of the immune system. The method includes contacting an immune cell with a modulator of HPK1, resulting in a change in a PGE2-modulated effect on the immune system. In one embodiment, the modulator increases expression or activity of HPK1 and decreases immune activity of the immune cell, for example, in some embodiments, the modulator increases expression or activity of HPK1 and decreases immune activity of the immune cell. In another embodiment, the modulator decreases expression or activity of HPK1 and increases immune activity of the immune cell, e.g., a T cell. In certain embodiments, the cell is a T cell and the increased immune activity comprises increased expression of at least one TH1 cytokine compared to a reference. In another embodiment of the invention, the cell is a T cell and the increased immune activity comprises increased expression of at least one TH1 cytokine (Il-2 or gamma-interferon) compared to a reference. In some embodiments, the PGE₂ activity is not substantially altered in non-immune system cells when the non-immune system cell is contacted with the modulator of HPK1. The modulator of HPK1 can, in some aspects, specifically bind HPK1. In certain aspects, the compound enhances a function selected from the group consisting of T cell effector function, natural killer cell function, and antigen presentation function. In some embodiments, the enhanced function is in a dendritic cell.

In another aspect, the invention further relates to a method of promoting anti-tumor immunity. The method includes administering to a subject a compound that inhibits HPK1 expression or activity, thereby increasing anti-tumor immunity of the subject. In certain embodiments of the invention, the compound is a small molecule. In some embodiments, the compound specifically binds to HPK1. In yet another embodiment of the invention, the compound enhances a function selected from the group consisting of T cell effector function, natural killer cell function, and antigen presentation function. In certain embodiments, the enhanced function is in a dendritic cell.

Another aspect of the invention relates to a transgenic mouse whose somatic and germ cells comprise a nucleic acid construct that disrupts the endogenous HPK1 sequence, the disruption resulting in the transgenic mouse having a level of HPK1 activity that is less than the level of HPK1 activity observed in a control mouse lacking the disruption, such that the transgenic mouse is homozygous for the disruption and lacks HPK1 polypeptide expression. In some embodiments, the transgenic mouse includes somatic and germ cells that are heterozygous for a nucleic acid construct that disrupts the endogenous HPK1 sequence of the transgenic mouse. In certain embodiments, the first exon of an endogenous HPK1 gene is disrupted.

Yet another aspect of the invention is a composition comprising a compound identified using any of the methods for identifying a compound that can modulate HPK1 activity as described herein. In certain embodiments, the composition can include a pharmaceutically acceptable excipient.

Other features and advantages of the invention will be apparent from the detailed description, drawings, and from the claims.

### DESCRIPTION OF THE DRAWINGS

FIG. 1 is a photographic representation of an, immunoblot of HPK1 from bone marrow derived dendritic cells (BMDCs). Lane 1: immature BMDCs, lane 2: matured with E. coli lipopolysaccharide (LPS), lane 3: BMDCs matured with Salmonella LPS.

FIG. 2A is a diagrammatic representation of the gene targeting strategy for generating HPK1-deficient mice. The top section is a diagram of a portion of the wild type murine HPK1 locus showing relevant restriction sites: Bam HI, Eco RI, Xho I, Xba I. Exons are shown as filled rectangles and the position of the 3′ flanking probe is indicated. The structure of the targeting vector (middle section) and the mutant locus (bottom section) are also shown.

FIG. 2B is a photographic representation of a PCR-based genomic analysis of HPK1^{+/+} (+/+), HPK1^{+/-} (+/−), and HPK1^{-/-} (−/−) mice.

FIG. 2C is a photographic representation of a Southern blot of EcoRI digested DNA from tail biopsies of littermates from crosses of HPK1^{+/-} mice.

FIG. 2D is a photographic representation of a Western blot analysis of protein lysates from mouse spleens using antibodies directed against HPK1. Lane 1: HPK1^{+/+} splenocytes, lane 2: HPK1^{-/-} splenocytes.

FIG. 3A is a graphic representation of a FACS plot of immature dendritic cells (iDC) stained using anti-CD80 antibody, wherein filled histograms depict HPK1^{+/+}, while the empty histograms represent HPK1^{-/-} BMDCs.

FIG. 3B is a graphic representation of a FACS plot of 48 hour LPS matured dendritic cells (mDC) stained using anti-CD80 antibody, wherein filled histograms depict HPK1^{+/+}, while the empty histograms represent HPK1^{-/-} BMDCs.

FIG. 3C is a graphic representation of a FACS plot of immature dendritic cells (iDC) stained using anti-CD86 antibody, wherein filled histograms depict HPK1^{+/+}, while the empty histograms represent HPK1^{-/-} BMDCs.

FIG. 3D is a graphic representation of a FACS plot of 48 hour LPS matured dendritic cells (mDC) stained using anti-CD86 antibody, wherein filled histograms depict HPK1^{+/+}, while the empty histograms represent HPK1^{-/-} BMDCs.

FIG. 3E is a graphic representation of a FACS plot of immature dendritic cells (iDC) stained using anti-I-ab antibody, wherein filled histograms depict HPK1^{+/+}, while the empty histograms represent HPK1^{-/-} BMDCs.

FIG. 3F is a graphic representation of a FACS plot of 48 hour LPS matured dendritic cells (mDC) stained using anti-I-ab antibody, wherein filled histograms depict HPK1^{+/+}, while the empty histograms represent HPK1^{-/-} BMDCs.

FIG. 3G is a graphic representation of a FACS plot of immature dendritic cells (iDC) stained using anti-CD11c antibody, where filled histograms depict HPK1^{+/+}, and the empty histograms represent HPK1^{-/-} BMDCs.

FIG. 3H is a graphic representation of a FACS plot of 48 hour LPS matured dendritic cells (mDC) stained using anti-CD11c antibody, where filled histograms depict HPK1^{+/+} and the empty histograms represent HPK1^{-/-} BMDCs.

FIG. 4A is a graphic representation depicting the results of experiments in which a mixed lymphocyte reaction (MLR) was performed for 48 hours. Shown in the Y-axis is a fixed T cell: variable dendritic cell ratios where the number of T cells is 2×10⁵ per well. +/+ (wild type) and −/− (HPK1^{-/-}) are immature dendritic cell controls. −/−LPS and +/+LPS refer to the HPK1^{-/-} and wild type dendritic cells matured in LPS, respectively. T cells plus ConA is a positive control for T cell stimulation.

FIG. 4B is a graphic representation depicting the results of experiments in which a MLR was performed for 96 hours. Shown in the Y-axis is a fixed T cell: variable dendritic cell ratios where the number of T cells is 2×10⁵ per well. +/+ (wild type) and −/− (HPK1^{-/-}) are immature dendritic cell controls. −/−LPS and +/+LPS are the HPK1^{-/-} and wild type dendritic cells matured in LPS, respectively. T cells plus ConA is a positive control for T cell stimulation.

FIG. 5A is a graphic representation of an in vitro cell migration assay measuring BMDC migration to a CCL-21 chemotactic gradient after 45 minutes of culture at 37° C. and 5% CO₂.

FIG. 5B is a graphic representation of an in vitro cell migration experiment assaying BMDC migration to a CCL-21 chemotactic gradient after 90 minutes of culture at 37° C. and 5% CO₂.

FIG. 5C is a graphic representation of an in vitro cell migration experiment assaying BMDC migration to a CCL-21 chemotactic gradient after 180 minutes of culture at 37° C. and 5% CO₂.

FIG. 5D is a graphic representation of an in vivo cell migration experiment assaying dendritic cell migration to both the popliteal and inguinal lymph nodes (assayed by FACS). Y axis: percentage of CFSE positive cells in the lymph nodes.

FIG. 6A is a representation of a Western blot of whole cell lysates stimulated with PGE₂ or by anti-CD3-mediated antibody crosslinking and probed with a monoclonal antibody directed against phosphotyrosine (anti-phosphotyrosine).

FIG. 6B is a representation of a Western blot of immunoprecipitates from cells stimulated as described for FIG. 6A, immunoprecipitated with anti-HPK1, and probed with anti-HPK1.

FIG. 6C is a representation of a Western blot of immunoprecipitates from cells stimulated as described for FIG. 6A, immunoprecipitated with anti-HPK1, and probed with Anti-pTyr.

FIG. 6D is a representation of a Western blot of immunoprecipitates from cells stimulated as described for FIG. 6A, immunoprecipitated with anti-HPK1, and assayed by IVK.

FIG. 7A is a representation of a phosphoimage of ³²P incorporated into the exogenous substrate (histone H2A, arrow) of cells treated as describe for FIG. 6A. The numbers under each lane represent the fold increase relative to the baseline kinase activity.

FIG. 7B is a representation of a Western blot of proteins as in FIG. 7A probed with anti-HPK1.

FIG. 8A is a representation of a schematic drawing of HPK1 structure depicting the domain structure of wild type and proline-deleted mutants (Δ proline).

FIG. 8B is a photographic representation of an IVK assay in which hemaglutinin-tagged (HA-tagged) wild type HPK1 or a proline-deleted mutant were transfected into a Jurkat cell line, stimulated as described for FIG. 6A, exogenous HPK1 immunoprecipitated using an anti-HA antibody and the catalytic activities assayed using an IVK assay.

FIG. 9 is a photographic representation of the results of an IVK assay in which cells stably expressing EP2 or EP4 were transiently transfected with HA-tagged HPK1, transfectants were untreated or stimulated by PGE₂ (as for FIG. 6A), exogenous HPK1 immunoprecipitated and the immunoprecipitates subjected to IVK assay.

FIG. 10 is a photographic representation of the results of an IVK assay in which cells were treated with phosphatase inhibitors, lysed, HPK1 immunoprecipitated, and HPK1 activity determined using IVK assay. CA is calyculin A, OK is okadaic acid, CSA is cyclosporine A, and Per is pervanadate.

FIG. 11 is a photographic representation of the results of IVK assays from experiments in which Jurkat cells were untreated or pretreated with H-89 (PKA inhibitor) and subsequently stimulated with CTX, DB (adenosine-3′,5′-cyclic monophosphate, N⁶, O², -Dibutyryl-, sodium salt), 8BM (adenosine-3′,5′-cyclic monophosphate, 8-bromo-, sodium salt), or forskolin (Fors), then HPK1 immunoprecipitated from cell lysates and activity determined using an IVK assay.

FIG. 12A is a diagrammatic representation of a comparison of the activation loop of HPK1 to other Ste20 family members. The gray box indicates conserved serine or threonine residues at the position 171. The consensus PKA site is indicated by a bar.

FIG. 12B is a photographic representation of the results of an immunoblot of an anti-phospho PKA substrate antibody that recognizes the arginine-based motif that is present at sites that are phosphorylated by PKA using cell extracts from control cells, CD3-treated cells, and PGE₂-treated cells.

FIG. 12C is a photographic representation of the results of an immunoblot of the HA-immunoprecipitates as in FIG. 12B probed with anti-HPK1.

FIG. 12D is a photographic representation of the results of an IVK assay in which HA-tagged wild type or point mutants of HPK1 (R168K, R169K, and S171A) were transfected into Jurkat cells, transfectants were untreated or stimulated by PGE₂ as described herein, exogenous HPK1 was then immunoprecipitated using an anti-HA antibody, and the catalytic activity of the immunoprecipitated HPK1 was determined using an IVK assay.

FIG. 13A is a graphic representation of assay data of relative IL-2 promoter activity in cells stimulated by anti-CD3 antibody and PMA or unstimulated cells. Y axis: vector is uninserted vector; C373A is Cbl-b is an ubiquitin ligase-defective mutant; C373A/2Y>F is a C373A mutant with tyrosine to phenylalanine mutations at the major TCR-induced phosphorylation sites, residues 665 and 709.

FIG. 13B is a graphic representation of IL-2 assay data of adjusted luciferase value of unstimulated transfectants subtracted from the stimulated value. Y axis is as in FIG. 13A.

FIG. 13C is a graphic representation of assay data of relative NFAT activity in cells stimulated by CD3 and PMA or unstimulated cells. Y axis is as in FIG. 13A.

FIG. 13D is a graphic representation of NFAT assay data of adjusted luciferase value of unstimulated transfectants subtracted from the stimulated value. Y axis is as in FIG. 13A.

FIG. 14A is a graphic representation of relative luciferase values reflecting NFAT/AP-1 expression of resting and anti-CD3/PMA-stimulated transfectants.

FIG. 14B is a photographic representation of an anti-Crk Western blot of anti-HA immunoprecipitated Crk proteins.

FIG. 15A is a representation of an autoradiogram of SDS-PAGE-resolved proteins associated with Crk fusion proteins.

FIG. 15B is a representation of a Western blot of Jurkat cell p95 (HPK1) protein pulled down by a GST-Crk SH3 domain or an SH3-defective W169L mutant of the GST-Crk SH3 domain and probed with an anti-HPK1 antibody.

FIG. 15C is a representation of a Western blot of proteins associated with Crk after immunoprecipitation. HPK1 as visualized using anti-HPK1 antibody.

FIG. 16A is a photographic representation of immunoprecipitated HPK1 subjected to kinase analysis. Lanes represent data from experiments using are vector alone, vector encoding wild type Cbl-b, and a ligase defective C373A Cbl-b mutant.

FIG. 16B is a photographic representation of immunoprecipitated HPK1 subjected to in vitro kinase analysis in the presence of ³²P y-ATP and histone H2A as exogenous substrate.

FIG. 16C is a photographic representation of anti-Cbl-b Western blot of whole cell lysates prepared from transfectants.

FIG. 16D is a photographic representation of an autoradiogram of ³²P-incorporated exogenous substrate (histone H2A) (upper panel). Lower panel: Anti-murine HPK1 Western blot of the immunoprecipitated HPK1.

FIG. 17A is a schematic representation depicting the domain structure of wild type and the kinase domain-deleted dominant interfering mutant (HPK1 Akin).

FIG. 17B (upper panel) is a graphic representation of relative NFAT/AP-1 expression in cells transfected with empty vector or vector encoding wild type HPK1 or the kinase domain-deleted HPK1 mutant and stimulated with anti-CD3/PMA as for the experiments depicted in FIG. 13. Open bars: unstimulated cells; shaded bars: stimulated cells. Lower panel: Anti-HA Western blotting to detect the expression level of HPK1 proteins.

FIG. 17C (upper panel) is a graphic representation of the relative NFAT/AP1 expression in Jurkat cells transfected with siRNA complementary to human HPK1 mRNA and stimulated with anti-CD3/PMA. Open bars: unstimulated cells; shaded bars: stimulated cells. Lower panel: Anti-HPK1 Western blot demonstrating the level of endogenous HPK1 expression.

FIG. 18A is a photographic representation of spleens from a 12-month-old HPK1^{-/-} mouse (right) and an age-matched C57BL/6 control.

FIG. 18B is a photomicrographic representation of a section the spleen of a 12 month old HPK1^{-/-} with splenomegaly mouse fixed in hematoxylin/eosin with 100× magnification.

FIG. 18C is a graphic representation of proliferation represented as ³H-thymidine uptake (cpm incorporated) of wild type C57BL/6 and HPK1 splenocytes untreated (empty bars) or stimulated with ConA (shaded bars) for 48 hours.

FIG. 18D is a graphic representation of IL-2 production by splenocytes from C57BL/6 and HPK1^{-/-}. Animals that were left untreated (empty squares) or were stimulated by either ConA (black bars) or by plate-bound anti-CD3 and soluble anti-CD28 for antibody-mediated crosslinking (gray bars).

FIG. 18E is a graphic representation of IL-2 production by wild type C57BL/6 or HPK1^{-/-} cultured splenic T cells primed for five days with either anti-CD3+CD28 antibody-mediated crosslinking or by ConA stimulation (empty bars). Primed cells were re-stimulated by ConA (black bars) or anti-CD3+CD28 Ab-mediated crosslinking (gray bars).

FIG. 18F is a graphic representation of the percentage of IL-2-producing CD4⁺ splenic T cells from wild type and HPK1^{-/-} CD4⁺ primed with anti-CD3 and anti-CD28 antibodies.

FIG. 19A is a photographic representation of results of an in vitro kinase assay (IVK) experiment in which RBC-excluded splenocytes were exposed to anti-CD3 and anti-CD28 antibody-mediated crosslinking or to PGE₂ for 5 minutes prior to lysis and immunoprecipitation with HPK1 antibodies. The IVK assay was performed in the presence of histone H2A, as the exogenous substrate. ³²P-incorporated into HPK1 was detected.

FIG. 19B is a photographic representation of the endogenous immunoprecipitated HPK1^{-/-} levels of the samples described in FIG. 19A.

FIG. 20A is a graphic representation of ³H-thymidine incorporation into wild type (WT) and HPK1^{-/-} (KO) cells subjected to an MLR with LPS-matured BMDCs in the presence (open bars) or absence (black bars) of PGE₂, then pulsed with ³H-thymidine.

FIG. 20B is a graphic representation of ³H-thymidine incorporation into wild type (WT) and HPK1^{-/-} (KO) cells subjected to an MLR with LPS-matured BMDCs expanded by anti-CD3 and anti-CD28 antibody mediated crosslinking in the presence (open bars) or absence (black bars) of PGE₂, then pulsed with ³H-thymidine.

FIG. 21A is a graphic representation of IL-2 levels assayed by ELISA in supernatants from ConA stimulated cells with or without PGE₂ and then re-stimulated with anti-CD3/anti-CD28 in the presence of GolgiStop™. Solid bars represent the number of IL-2 positive cells in the absence of PGE₂ and open bars represent IL-2 positive cells in the presence of PGE₂. T cells are indicated by WT and KO indicated HPK1^{-/-} cells.

FIG. 21B is a graphic representation as in FIG. 21A in which intracellular IL-2 was assayed.

FIG. 22A is a graphic representation of IL-2 production in T cells stimulated with anti-CD3 and anti-CD28 in a primary stimulation experiment.

FIG. 22B is a graphic representation of IFN-γ (IFN-g) production in T cells stimulated with anti-CD3 and anti-CD28 in a primary stimulation experiment.

FIG. 22C is a graphic representation of IL-4 production in T cells stimulated with anti-CD3 and anti-CD28 in a primary stimulation experiment.

FIG. 23A of IFN-γ production in T cells stimulated with anti-CD3 and anti-CD28 in a in a secondary stimulation experiment after a 5 day rest and restimulation.

FIG. 23B of IL-2 production in T cells stimulated with anti-CD3 and anti-CD28 in a primary stimulation experiment in a secondary stimulation experiment after a 5 day rest and restimulation.

FIG. 23C is a graphic representation of TCR-induced proliferation of HPK1+/+ or HPK1−/− cells that were stimulated with TCR-crosslinking for 3 days and proliferation measured by thymidine incorporation.

FIG. 23D is a graphic representation of TCR-induced proliferation of HPK1+/+ or HPK1−/− cells that were stimulated with concanavalin A for 3 days and proliferation measured by thymidine incorporation.

FIG. 24A is a graphic representation IL-2 production from 3 day stimulated TCR-stimulated cells that were collected prior to thymidine addition and assayed for IL-2 levels by ELISA.

FIG. 24B is a set of reproductions of raw FACS scatter of plots T cells (HPK1^{+/+}) and HPK1^{-/-} cells, were stimulated with anti-CD3 anti-CD28, and detected by anti-CD4 staining in the presence or absence of PGE₂ for 48 hours with GolgiStop™ added for the last 6 hours, permeabilized and stained for IL-2.

FIG. 24C is a graphic representation of FACS results as in FIG. 24B depicting the percentage of CD4+IL-2+ cells. Black bars represent the number of IL-2 positive cells without PGE₂ and white bars represent IL-2 positive cells with PGE₂.

FIG. 24D is a graphic representation of proliferation assayed by thymidine incorporation into cells stimulated as in FIG. 24A. Black bars represent the number of IL-2 positive cells without PGE₂ and white bars represent IL-2 positive cells with PGE₂. Error bars represent the standard deviation from the average of three or five different experiments respectively.

FIG. 25A is a graphic representation of proliferation of cells in the presence of anti-CD3 in titration experiments in which cells were added to the plates coated with various concentrations of anti-CD3 with a fixed concentration of anti-CD28 (1 ug/ml) and were incubated for 3 days in the presence of PGE₂. Dark bars are results using wild type cells and light grey bars are results using HPK1^{-/-} cells.

FIG. 25B is a graphic representation of proliferation of cells in the presence of anti-CD3 (0.75 μg/ml or 3.0 μg/ml) in experiments in which cells were added to the plates coated with various concentrations of anti-CD3 with a fixed concentration of anti-CD28 (1 ug/ml) and were incubated for 3 days in the presence of PGE₂. Dark bars are results using wild type cells and light grey bars are results using HPK1^{-/-} cells.

FIG. 26A is reproduction of FACS plots of fluorescence intensity of T cells (HPK1+/+ or HPK1^{-/-}) that have been stimulated via TCR crosslinking, in the presence or absence of PGE₂ for 3 days then stained for CD4 and CD25.

FIG. 26B is a reproduction of FACS plots of fluorescence intensity of T cells (HPK1^{+/+} or HPK1^{-/-}) that have been stimulated via TCR crosslinking, in the presence or absence of PGE₂ for 3 days then the CD4 gated population was subsequently stained with CD25 and Foxp3.

FIG. 27A is a reproduction of a set of FACS fluorescence intensity plots of T cells (HPK1^{+/+} and HPK1^{-/-}) stimulated as for the experiment of FIG. 26, and stained for 7-amino-actinomycin D (7AAD) and annexin V and the 7AAD negative population, which represents living cells, was assayed for annexin V staining.

FIG. 27B is a graphic representation of the percent of annexin V positive cells stimulated as for FIG. 25B and stained for annexin V and the percent of annexin positive cells is plotted. Black squares represent results using wild type cells in the absence of PGE₂, white squares represent the results using wild type cells in the presence of PGE₂, black circles represent results using HPK1^{-/-} cells in the absence of PGE₂, and the white circles represent the results using HPK1^{-/-} cells in the presence of PGE₂.

FIG. 28A is a graphic representation of data monitoring tumor growth in 3LL cells that were injected subcutaneously into HPK1^{-/-} and wild type cells.

FIG. 28B is a graphic representation of data monitoring tumor volume in 3LL cells that were injected subcutaneously into HPK1^{-/-} and wild type mice that were treated with Cox2 inhibitor 3 times weekly after 3LL cell injection and monitored as in 28A. Tumor size represents the tumor area as a product of the small and large diameters.

FIG. 28C is a graphic representation of data surveying the total number of foci in lung in mice in which 3LL cells were injected intravenously into mice in the presence of Cox-2 inhibitor as in 28B. Mice were sacrificed 14 days later and their lungs were stained with hematoxylin and eosin (H&E). The histogram represents the total number of foci per lung.

### DETAILED DESCRIPTION

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It has been found that HPK1 has a unique expression pattern in dendritic cells. It has also been determined that LPS-matured bone marrow derived dendritic cells (BMDCs) lacking HPK1 are functionally superior to their wild type counterpart. For example, matured HPK1^{-/-} BMDCs express higher levels of maturation markers, migrate more efficiently to the regional lymph nodes, and stimulate T cells more effectively. Thus, decreasing HPK1 expression or activity in BMDCs is useful, e.g., for increasing an immune response. Conversely, increasing HPK1 expression or activity in BMDCs can decrease an undesirable immune response, e.g., in autoimmune disease such as rheumatoid arthritis.

HPK1 becomes catalytically active in response to stimulation by physiological concentrations of prostaglandin E2 (PGE₂). Thus, HPK1 plays a role in suppression of the immune response, e.g., in suppression of an anti-tumor immune response. It has been found that this stimulation results in increased activity of protein kinase A, which phosphorylates ser171 of HPK1. Thus, inhibiting PGE₂ can decrease PKA activity, which in turn, can decrease activation of HPK1. Conversely, increasing PGE₂ activity or increasing PKA activity are methods for increasing HPK1 activity.

PGE₂ is secreted by tumor cells and by activated antigen presenting cells (APCs) and has been implicated as a major cause of inflammatory and cancer mediated immune suppression. The immunosuppressive effects elicited by PGE₂ hinder efficient treatment of infections and may impair successful immunotherapeutic treatment of certain cancers. Via a poorly defined mechanism, PGE₂ suppresses the immune system primarily through inhibiting T cell proliferation and interleukin 2 (IL-2) production. It is demonstrated herein that murine T cells genetically lacking hematopoietic progenitor kinase 1 (HPK1) exhibit resistance to PGE₂-mediated inhibition, that is, a decrease in inhibitory effects of PGE₂. Specifically, HPK1^{-/-} T cells produce higher levels of IL-2 and proliferate better than wild type T cells in the presence of PGE₂. This suppression is, in part, mediated by a novel resistance of HPK1^{-/-} T cells to PGE₂-induced apoptosis. Thus, the invention also relates to inhibiting immune suppression by decreasing HPK1 expression or activity.

The invention also relates to the finding that tumors develop more slowly in HPK1^{-/-} animals and there are more lymphocytic infiltrates found in engrafted tumors in HPK1^{-/-} mice. Thus, tumor development can be inhibited and lymphocytic activity increased by decreasing HPK1 expression or activity.

The invention further relates to identification and use of compounds that can modulate HPK1 expression or activity, including compounds that bind to HPK1 or a bind to a molecule that modulates HPK1.

Screening Assays

Aspects of the invention provide methods (also referred to herein as “screening assays”) for identifying modulators, i.e., candidate compounds (e.g., proteins, peptides, peptidomimetics, peptoids, small molecules or other drugs) that have a stimulatory or inhibitory effect on HPK1 expression or HPK1 activity. Compounds thus identified can be used to modulate the activity of HPK1 in a therapeutic protocol or to elaborate the biological function of HPK1, e.g., can be sold commercially for such uses.

The invention provides assays for screening test compounds for their ability to modulate HPK1 expression or activity, e.g., by modulating Cbl-b expression or activity or PKA expression or activity. Such compounds can act indirectly to modulate HPK1 activity e.g., by modulating Cbl-b or PKA expression or activity, or by directly interacting with HPK1, e.g., by binding to HPK1 and interfering with PKA-mediated phosphorylation of PKA.

In a particular embodiment, an assay is a cell-based assay in which a cell that expresses an HPK1 protein or biologically active portion thereof is contacted with a test compound, and the ability of the test compound to modulate HPK1 activity is determined. Determining the ability of the test compound to modulate HPK1 activity can be accomplished by monitoring consequential activities of HPK1 activity including, without limitation, T cell activation, dendritic cell maturation, or dendritic cell migration. Such assays are described infra. The cell can be of mammalian origin, e.g., human, murine, or rat.

The ability of the test compound to modulate HPK1 binding to a compound, e.g., a PKA can also be evaluated according to the methods of the invention. This can be accomplished, for example, by coupling the compound, e.g., PKA, with a radioisotope or enzymatic label such that binding of the compound can be determined by detecting the labeled compound in a complex. Alternatively, an HPK1 can be coupled with a radioisotope or enzymatic label to monitor the ability of a test compound to modulate HPK1 binding to, e.g., PKA in a complex. For example, compounds can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

The ability of a compound to interact with a protein, e.g., HPK1, PKA, or Cbl-b, with or without the labeling of any of the interactants, can be evaluated by this method. For example, the interaction of a compound with an HPK1, PKA, or Cbl-b can be detected, e.g., using a microphysiometer, without the labeling of either the compound or the protein (McConnell et al. (1992, Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and the protein.

In general, cell-free assays involve preparing a reaction mixture of the protein and the test compound under conditions and for a time sufficient to allow the two components to interact and bind, thus forming a complex that can be removed and/or detected.

In yet another embodiment, a cell-free assay is provided in which a protein or biologically active portion thereof is contacted with a test compound, and the ability of the test compound to bind to the protein or biologically active portion thereof is evaluated. Biologically active portions of the proteins to be used in assays of the present invention include fragments that participate in interactions with non-HPK1, Cbl-1, or PKA molecules, e.g., fragments with high surface probability scores.

Soluble and/or membrane-bound forms of isolated proteins or biologically active portions thereof can be used in the cell-free assays of the invention. When membrane-bound forms of the protein are used, it may be desirable to utilize a solubilizing agent. Non-limiting examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylammonio]-1-propane sulfonate (CHAPS), 3-[(3-cholamidopropyl)-dimethylammonio]-2-hydroxypropane-1-sulfonate (CHAPSO), or N-dodecyl=N,N-dimethyl-3-ammonio-1-propane sulfonate.

The interaction between two molecules can also be detected, e.g., using fluorescence energy transfer (FET) (see, for example, U.S. Pat. Nos. 5,631,169 and 4,868,103; and fretimaging.org/mcnamaraintro.html). A fluorophore label on the first, ‘donor’ molecule is selected such that the emitted fluorescent energy of the donor is absorbed by a fluorescent label on a second, ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art, e.g., using a fluorimeter.

In another embodiment, determining the ability of the protein to bind to a target molecule can be accomplished using real-time Biomolecular Interaction Analysis (BIA) (e.g., Sjolander and Urbaniczky, 1991, Anal. Chem. 63:2338-2345 and Szabo et al., 1995, Curr. Opin. Struct. Biol. 5:699-705). “Surface plasmon resonance” or “BIA” detects biospecific interactions in real time, without labeling any of the interactants (e.g., BIAcore). Changes in the mass at the binding surface, indicative of a binding event, result in alterations of the refractive index of light near the surface (the optical phenomenon of surface plasmon resonance (SPR)), resulting in a detectable signal that can be used as an indication of real-time reactions between biological molecules.

In a particular embodiment, the protein or the test compound is anchored onto a solid phase. The target gene product/test compound complexes anchored on the solid phase can be detected at the end of the reaction. In general, the target gene product can be anchored onto a solid surface, and the test compound (which is not anchored) can be labeled, either directly or indirectly, with detectable labels discussed herein.

The protein, an antibody that binds to the protein, or a target molecule that binds to the protein, may be immobilized to facilitate separation of complexed from uncomplexed forms of one or both of the proteins, as well as to accommodate automation of the assay. Binding of a test compound to a protein, or interaction of a protein with a target molecule in the presence and absence of a candidate compound, can be accomplished in any vessel suitable for containing the reactants. Non-limiting examples of such vessels include microtiter plates, test tubes, and micro-centrifuge tubes. In one embodiment, a fusion protein is provided that adds a domain that allows one or both of the proteins to be bound to a matrix. For example, glutathione-S-transferase/fusion proteins or glutathione-S-transferase/target fusion proteins can be adsorbed onto glutathione Sepharose® beads (Sigma Chemical, St. Louis, Mo.) or glutathione derivatized microtiter plates, which are then combined with the test compound or the test compound and either the non-adsorbed target protein (e.g., PKA) or HPK1 protein, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes are dissociated from the matrix, and the level of protein (e.g., HPK1, PKA, or Cbl-b) binding or activity is determined using standard techniques.

Other techniques for immobilizing an HPK1, PKA, or Cbl-b protein or a target molecule on matrices include using conjugation of biotin and streptavidin. Biotinylated protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). To conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the immobilized component (the antibody, in turn, can be directly labeled or indirectly labeled with, e.g., a labeled anti-Ig antibody).

In certain embodiments, this assay is performed utilizing antibodies reactive with an HPK1, PKA, or Cbl-b protein or target molecules, but which do not interfere with binding of the protein to its target molecule. Such antibodies can be derivatized to the wells of the plate, and unbound target or protein trapped in the wells by antibody conjugation. Methods for detecting such complexes, in addition to those described above for the GST-immobilized complexes, include immunodetection of complexes using antibodies reactive with the protein or target molecule, as well as enzyme-linked assays that rely on detecting an enzymatic activity associated with the protein or target molecule.

Alternatively, cell-free assays can be conducted in a liquid phase. In such an assay, the reaction products are separated from unreacted components, by any of a number of standard techniques, including but not limited to: differential centrifugation (see, for example, Rivas et al., 1993, Trends Biochem. Sci. 18:284-287); chromatography (gel filtration chromatography, ion-exchange chromatography); electrophoresis (see, e.g., Ausubel et al., eds. Current Protocols in Molecular Biology 1999, J. Wiley: New York.); and immunoprecipitation (see, for example, Ausubel et al., eds. (1999, Current Protocols in Molecular Biology J. Wiley: New York). Such resins and chromatographic techniques are known to one skilled in the art (see, e.g., Heegaard, 1998, J. Mol. Recognition. 11: 141-8; Hage et al., 1997 J. Chromatogr. B. Biomed. Sci. Appl. 699:499-525). Further, fluorescence energy transfer may also be conveniently utilized, as described herein, to detect binding without further purification of the complex from solution.

In another embodiment, the assay includes contacting an HPK1 protein, PKA, of Cbl-b or biologically active portion thereof with a known compound that binds to the protein to form an assay mixture, contacting the assay mixture with a test compound, and determining the ability of the test compound to interact with the protein, wherein determining the ability of the test compound to interact with the protein includes determining the ability of the test compound to preferentially bind to the protein or biologically active portion thereof, or to modulate the activity of the protein, as compared to the known compound.

For the purposes of this discussion, target proteins are cellular and extracellular macromolecules are referred to herein as “binding partners,” that interact with an HPK1. Examples include, without limitation, SH2/SH3 domain-containing adaptor proteins that bind to the polyproline-rich region of HPK1 e.g., Crk, Crk-L, Grb-2, Nck, and Grap/Gad), scaffolding proteins that bind to the polyproline-rich region of HPK1 (e.g., Clnk and HIP-55); tyrosine kinases that bind to the polyproline-rich region of HPK1 (e.g., Abl and Src); serine/threonine kinases that bind to the CITRON homology domain of HPK1 (e.g., MEKK1 and MLK), guanine nucleotide exchange factors specific for the member of Rho family of GTPases (e.g., αPIX and βPIX); and actin.

Additional candidate binding partners include proteins identified as co-precipitating with HPK1 isolated from cells, A kinase anchor protein (AKAP), or proteins that bind to a Cbl-b. Examples of Cbl-b binding partners include Crk and Crk-L SH2/SH3 domain-containing adaptor proteins. Other suitable Cbl-b binding proteins include, CIN85 (which is a CD2AP family member that binds to Cbl-b), Eps15 (an adaptor protein that plays a role in receptor endocytosis), ZAP-70, and EGF receptor. ZAP-70 and EGF receptor are tyrosine kinases that bind to the N-terminal TKB domain of Cbl-b and are ubiquitinated by the RING-type ligase domain of Cbl-b. Compounds that disrupt such interactions are useful in regulating the activity of the protein. Such compounds include, but are not limited to, molecules such as antibodies, peptides, and small molecules. In an alternative embodiment, the invention provides methods for determining the ability of the test compound to modulate the activity of an HPK1 protein through modulation of the activity of a downstream effector of an upstream molecule (e.g., Cbl-b). For example, the activity of the effector molecule on an appropriate target can be determined, or the binding of the effector to an appropriate target can be determined, as previously described. In general, the assay includes both a determination of the effect of a compound on the upstream effector molecule (e.g., Cbl-b or PKA) and the activity of HPK1.

In general, to identify compounds that interfere with the interaction between the target gene product and its cellular or extracellular binding partner(s), a reaction mixture containing the protein and the binding partner is prepared under conditions and for a time sufficient, to allow the two products to form complex. To test an inhibitory agent, the reaction mixture is provided in the presence and absence of the test compound. The test compound can be initially included in the reaction mixture, or can be added at a time subsequent to the addition of the target gene and its cellular or extracellular binding partner. Control reaction mixtures are incubated without the test compound or with a placebo. The formation of any complexes between the protein and the cellular or extracellular binding partner is then detected. The formation of a complex in the control reaction, but not in the reaction mixture containing the test compound, indicates that the compound interferes with the interaction of the protein and the interactive binding partner. Additionally, complex formation within reaction mixtures containing the test compound and normal protein (e.g., HPK1, PKA, or Cbl-b) can also be compared to complex formation within reaction mixtures containing the test compound and mutant target gene product. This comparison is useful in those cases wherein it is desirable to identify compounds that disrupt interactions of mutant but not normal target gene products.

These assays can be conducted in a heterogeneous or homogeneous format. Heterogeneous assays involve anchoring either the protein (e.g., HPK1, PKA, or Cbl-b) or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the protein and the binding partner, e.g., by competition, can be identified by conducting the reaction in the presence of the test substance. Alternatively, test compounds that disrupt preformed complexes, e.g., compounds with higher binding constants that displace one of the components from the complex, can be tested by adding the test compound to the reaction mixture after complexes have been formed.

In another aspect, the invention is directed to a combination of two or more of the assays described herein. For example, a compound that modulates the expression or activity of a molecule that regulates HPK1 expression or activity is also assayed for the ability to modulate HPK1 activity. In another example, a compound that modulates PKA expression or activity is also assayed for its ability to modulate HPK activity.

Useful assays also include assays that confirm a predicted effect of a compound that can modulate (increase or decrease) HPK1 expression or activity. Examples of such assays, described herein and known in the art include, without limitation, assays of PKA, Cbl-1b, indicia of dendritic cell maturation, T cell proliferation, IL-2 production, TNF secretion, IFN-γ, B cell proliferation, synthesis of an immunosuppressive cytokine, apoptosis of T cells, assay of T cell resistance to PGE₂-mediated suppression of T cell receptor-induced activation, apoptosis of a tumor cell, total cytokine profiling, apoptosis of dendritic cells, activation of dendritic cells, T cell priming and activation by antigen presenting cells or by TCR cross linking, T cell and dendritic cell migration. Such assays can be conducted in vivo and/or in vitro.

In some cases, the compound is further tested in an animal model. Examples of useful animal models are described infra.

Agents identified by the screening assays described herein can be used for treatments as described herein.

Test Compounds

Aspects of the present invention encompass compounds that directly or indirectly modulate expression or activity of an HPK1 protein. In some cases, the compound modulates expression or activity of a cAMP-activated protein kinase A (PKA) or other molecule that is in a cascade that regulates HPK1 activity. As used herein, the term “modulate” includes increasing or decreasing expression (e.g., RNA or protein) or activity. The increase or decrease in expression or activity is generally determined by comparison to a control or established reference.

An agent can, for example, be a small molecule. Such small molecules include, but are not limited to, peptides (including peptides that can cross a cell membrane (cell permeable peptide)), peptidomimetics (e.g., peptoids), amino acids, amino acid analogs, polynucleotides (e.g. an anti-sense nucleic acid or a short interfering RNA (siRNA)), polynucleotide analogs, nucleotides, nucleotide analogs, non-nucleic acid organic compounds, inorganic compounds (including heteroorganic and organometallic compounds). In general, such small molecules have a molecular weight less than about 10,000 grams per mole, e.g., a molecular weight less than about 5,000 grams per mole, a molecular weight less than about 1,000 grams per mole, a molecular weight less than about 500 grams per mole, and salts, esters, and other pharmaceutically acceptable forms of such compounds.

Larger compounds are also useful in the invention. For example, antibodies that specifically bind to an HPK1, or HPK1-binding fragments of such antibodies, are useful for modulating the activity of HPK1. Methods of facilitating entry of such compounds into a cell are known in the art. For example, Chariot™ protein transfection (Active Motif, Carlsbad, Calif.) can be used in vitro or in vivo to facilitate entry of whole antibody or a fragment of an antibody or other compound into a target cell. In one example, the compound is an antibody or a Fab or other binding fragment that targets the ATP binding site in the kinase domain of HPK1. A polyclonal antibody such as the anti-HPK1 AB#2 described in (Kiefer et al., 1996, EMBO J. 15:7013) blocks ATP access to HPK1 and render HPK1 catalytically inactive, thereby decreasing HPK1 activity. A monoclonal antibody can also be used and in general has higher affinity for the ATP site of HPK1; a monoclonal antibody is a reproducible resource. In addition, a useful compound for modulating HPK1 activity can be constructed using a monoclonal antibody that specifically binds to the ATP binding site of HPK1 or other site that affects activity, and the cDNA encoding at least the CDR (complementarity determining region) can be used to generate a chimeric single chain molecule that maintains target specificity (against HPK1), but can be expressed as transgene. Once synthesized and isolated, such a “single chain Ig” directed against the HPK1 ATP binding site can be delivered via using means known in the art, including, without limitation, transfection, and viral-mediated transduction.

Compounds that are pseudo-substrates for HPK1 are useful, e.g., for decreasing HPK1 activity. Such compounds can be identified using methods known in the art, for example, by determining the kinase motif preferred by HPK1 using, e.g., the dual-oriented degenerate peptide libraries technique (Hutti et al., 2004, Nat. Methods 1:27). Once the kinase motif is determined, databases are searched for proteins that contain that motif. Such proteins are then further examined for their ability to act as a substrate for HPK1, e.g., in a cell that is genetically engineered to express both HPK1 and the putative HPK1 substrate, in a system in which the putative substrate is knocked out (e.g., by expression of an siRNA targeted to a sequence encoding the substrate). Following identification of the kinase motif in the substrate, which can be accomplished, e.g., by analyzing the sequence, a peptide fragment corresponding to the kinase motif sequence is synthesized and can be used to inhibit HPK1 activity. If necessary, the fragment can also be modified to enable it to be membrane permeable, or it can be delivered using methods that facilitate entry into the cell. Such fragments compete with endogenous substrate for HPK1-mediated phosphorylation, and can be used to inhibit such phosphorylation. If an auto-inhibitory domain is identified in HPK1, a similar strategy can be employed to compete with that domain. Other compounds that can function as competitive inhibitors of ATP binding to a PKA kinase domain are also useful.

The compounds of the present invention can be obtained by any of the numerous approaches in combinatorial library methods known in the art, including, e.g., (biological libraries) peptoid libraries (libraries of molecules having the functionalities of peptides, but with a novel, non-peptide backbone that are resistant to enzymatic degradation but that nevertheless remain bioactive; see, e.g., Zuckermann et al. (1994, J. Med. Chem. 37:2678-85)), spatially addressable parallel solid phase or solution phase libraries) synthetic library methods requiring deconvolution) the “one-bead one-compound” library method and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art (DeWitt et al., 1993, Proc. Natl. Acad. Sci. U.S.A. 90:6909; Erb et al., 1994, Proc. Natl. Acad. Sci. USA 91:11422; Zuckermann et al., 1994, J. Med. Chem. 37:2678; Cho et al. 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233).

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner, U.S. Pat. No. 5,223,409), spores (Ladner U.S. Pat. No. 5,223,409), plasmids (Cull et al. (1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. 87:6378-6382; Felici, 1991, J. Mol. Biol. 222:301-310; Ladner, supra.). Commercial companies and libraries made available by such companies can also be used, e.g., to design and identify suitable compounds (e.g., RTI International, Research Triangle Park, N.C.).

In some cases, a compound that can modulate HPK1 specifically binds to its target molecule. By “specifically binds” is meant a molecule that binds to a particular entity, e.g., an HPK1 polypeptide in a sample, but that does not substantially recognize or bind to other molecules in the sample, e.g., a biological sample, that includes the particular entity, e.g., an HPK1 polypeptide.

Assaying the Modulation of HPK1

In certain aspects of the invention, HPK1 activity is assayed. Such activity can be assayed directly or indirectly and assays are described throughout the present application. Indirect assays include, for example, migration assays. In one such representative migration assay, immature or LPS matured dendritic cells are labeled with CFSE and then injected into the footpad of a wild type mouse. After various periods of time, the regional lymph nodes of the mouse are harvested and the lymph node cells are analyzed using FACS analysis to identify the CFSE positive population. The CFSE positive cells represent cells that have migrated. The lymph nodes are harvested and analyzed at different time points to determine speed and efficiency of dendritic cell migration in the presence and absence of a test compound.

Another method of assaying HPK1 activity is to analyze the expression or activity of cytokines that are affected by HPK1 activity. For example, cytokine production is assayed in dendritic cells from animals that were administered a test compound and compared to cytokine production in dendritic cells from untreated controls. Similarly, cytokine skewing of dendritic cells can be assayed by measuring cytokines that are produced by Th1 and Th2 cells in animals that were administered a test compound. Cytokine skewing is related to the process by which an activation signal induces a certain cytokine profile (mainly in APCs) that cause naïve T cells to either become Th1 or Th2 in phenotype. Cytokine skewing refers to APCs producing more of one set of cytokines that favor the development of either the TH 1 or the TH2 sub-population. Nonlimiting examples of cytokines that can be tested are interferon gamma (IFN-gamma), interleukin (IL)-2, lymphotoxin (LT) and granulocyte-macrophage colony-stimulating factor (GM-CSF) and IL-6 for Th1 cells and IL-3, IL-4, IL-5, IL-10 and IL-13 for Th2 cells. The cytokine measurements are compared to those in untreated cells. Induction of Th1 or Th2 cytokines by immune cells in the presence of a test compound indicates that the test compound is a candidate compound for inhibiting HPK1 expression or activity. Cytokines can be assayed using methods known in the art including, e.g., enzyme-linked immunosorbent assay (ELISA) or using flow cytometry (using a FACS), or by intracellular staining of specific cytokines.

In general, when an immune response is assayed by analyzing a T cell response, such as the production of specific cytokines, the test sample is compared to a reference that can be a predetermined baseline values or set of values or a control that is assayed in conjunction with the test sample(s). For example, a reference for testing immune response can be T cells in which there is no stimulation of an immune response. In general in such studies, a primary stimulation sample is included in which a test sample is stimulated once (e.g., by administering to an animal a compound that is an activator of TCR or administering ConA to a sample in vitro. Optionally, a secondary response sample is also assayed. In a secondary response sample, a second stimulus is administered to an animal about 3 to 5 weeks after administration of the first stimulus.

PKA Binding and Activity

In certain embodiments, a compound that affects HPK1 activity is a compound that modulates PKA expression or activity. PKA expression can be assayed using methods known in the art, e.g., Northern blot, Western blot, quantitative mass spectroscopy, or immunoassay (e.g., enzyme-linked immunosorbent assay; ELISA). A compound that decreases PKA expression or activity is a candidate compound for decreasing HPK1 activity. A compound that increases PKA expression or activity is a candidate for increasing HPK1 activity. In such experiments, a cultured cell or animal that can express PKA is contacted with a test compound. The cell or a sample from the animal is assayed for PKA expression and compared to a corresponding sample that was not contacted with the test compound (a control). A difference in the expression of PKA in a sample contacted with the test compound compared to the control indicates that the test compound can affect HPK1 activity.

In some cases, PKA activity is assayed. Methods of measuring PKA activity include any known assay, such as assaying phosphorylation of a ser171 in an HPK1 or HPK1 peptide containing ser171 of HPK1. Essentially, lysates from cells for PKA kinase activity assays are reacted with HPK1 activation loop peptide (DFGISAQIGATLARRLSFIGTPYWMAPE; SEQ ID NO:1) so that PKA can phosphorylate the peptide substrate. Mutant peptide with serine to alanine mutation at residue 171 can be used as a control. The assay is performed in the presence or absence of PKA inhibitor to control for the non-specific phosphorylation of the preferred PKA site on the peptide. After the phosphorylation is complete, the peptide is immobilized, and to determine the degree of phosphorylation, the peptide is immunoblotted using an antibody that recognizes only the phosphorylated form of PKA substrate. The degree of phosphorylation reflects the relative kinase activity in each sample tested. Other methods of assaying PKA include, without limitation, StressXpress PKA Kinase Expression Activity Kit (Stressgen, Victoria, BC, Canada) PKA Assay Kit (Molecular Devices, Sunnyvale, Calif.); QTL Lightspeed™ protein kinase A (PKA) Assay Kit (QTL Biosystems, Santa Fe, N. Mex.). Such assay systems can also be modified. For example, the StressXpress system can be modified so that the peptide substrate provided by the assay kit can be swapped with the peptide corresponding to the activation loop of HPK1. A peptide sequence that includes the activation loop with a serine to alanine mutation at S171 is useful as a negative control. In another non-limiting example of a PKA assay, PKA is immunoprecipitated from the cell or tissue to be assayed, and an in vitro kinase assay is performed by incubating immunoprecipitated PKA in the presence of ³²P γ-ATP, and an HPK1 peptide for 10 minutes at 30° C. The reaction is then terminated by adding 100 μl of 75 mM H₃PO₄. The supernatant from the reaction is collected and spotted onto P-81 phosphocellulose paper. Free γ ³²P-ATP is separated from the labeled substrate by washing four times for 5 minutes each in 75 mM H₃PO₄. The P-81 papers are dried and radioactivity incorporated into syntide-2 is determined by scintillation counting.

PKA assays can be combined with other methods described herein to identify compounds that modulate HPK1 activity. For example, the ability of a compound that modulates PKA activity can also be assayed for its ability to affect dendritic cell migration. A compound that decreases PKA activity and increases dendritic cell migration is a compound that decreases HPK1 activity. A compound that increases PKA activity and decreases dendritic cell migration is a compound that increases HPK1 activity.

Methods of Assaying Dendritic Cell Maturation and Activity

Dendritic cells are cellular bridges that couple the innate and adaptive immune systems. Dendritic cell maturation is governed by a unique set of functional and phenotypic changes, both of which are orchestrated by a highly integrated network of biochemical signals that are initiated upon the binding of cytokines and pathogen-derived antigens to their respective receptors. Upon antigen uptake, dendritic cells transition from an immature to a mature state and move from peripheral tissues to T cell-rich areas of the regional draining lymph nodes. Dendritic cells then, in a synchronized manner, up-regulate the transcription of co-stimulatory receptors and other pertinent signaling molecules involved in lymphocyte activation and trafficking. As a result, a phenotypically mature dendritic cell is able to up regulate functionally unique maturation markers such as the co-stimulatory molecules CD80 (B7.1) and CD86 (B7.2) as well as CD83, MHC class II, and CCR7 (Chen et al., 1999, Oncogene 18:7370), and becomes proficient in moving to the draining lymph nodes to stimulate T cells and catalyze the adaptive immune system.

The invention described herein includes methods of identifying compounds that modulate dendritic cell activity (e.g., activity of bone marrow derived dendritic cells (BMDCs)) via modulation of HPK1. Methods of assaying modulation of BMDC activity include, but are not limited to, assaying the expression or activity of dendritic cell maturation markers, e.g., CD80, CD86, CD83, MHC class II, and CCR7. Methods of assaying the activity of these molecules are known in the art. DC maturation markers such as CD80, CD86, CD83, and MHC class II are identified as adhesion molecules that function as co-receptors that facilitate cell-cell interaction by increasing avidity of the immunological synapse formed between antigen presenting cells such as DC and effector cells such as T cells. Up regulation of these molecules is an indicator that there is a corresponding increase in function (i.e., increased avidity if there are more adhesion molecules). Similarly, cells that up regulate chemotactic receptor for CCL-19/CCL-21 migrate more rapidly towards CCL-21 concentration gradient. The expression of these molecules can be determined by FACS analysis using labeled antibodies that are specific for each molecule to be detected. These antibodies are available from commercial sources (e.g., Pharmingen, San Jose, Calif.). Cytokine levels can generally be assayed using commercially available kits (e.g., from R&D Systems, Inc., Minneapolis, Minn.; Qiagen, Valencia, Calif.; BD Biosciences, Bedford, Mass.;), and Pierce Biotechnology (Endogen®; Rockville, Ill.). Accordingly, compounds that increase dendritic cell maturation are compounds that increase the expression of at least one dendritic cell marker in a cell. Compounds that increase dendritic cell maturation are candidate compounds for decreasing HPK1 expression or activity.

In certain embodiments of the invention, a test compound is administered to a cultured cell or to an animal and the migration or dendritic cells or the induction of one or more maturation markers is assayed. A change in the migration (e.g., the rate or number of migrating cells) or expression of a maturation marker indicates that the test compound is a candidate compound for modulating HPK1 activity.

In some cases, maturation of dendritic cells is induced. Methods for inducing dendritic cell maturation are known in the art and include without limitation, contacting a dendritic cell with one or more of any of the toll-like receptor (TLR) ligands such as lipopolysaccharide (LPS; e.g., about 5 μg/ml, Sigma, St. Louis, Mo.), poly I:C (e.g., about 12 μg/ml, Sigma, St. Louis, Mo.), PGE₂ (e.g., about 1 μg/ml, Sigma), or cytokine mixture (TNF-α, 5 ng/ml; rhIL-1β, about 5 ng/ml; and rhIL-6, and about 150 ng/ml; R&D Systems). Compounds of the invention that decrease HPK1 expression or activity can increase the rate or magnitude of dendritic cell maturation. Magnitude of dendritic cell maturation can be assayed, for example, by assaying the levels of cytokines. A compound that alters cytokine levels from normally observed levels in a manner associated with increased maturation is indicative of induction of maturation. An enhanced magnitude of dendritic cell maturation could be due to an increase in the number of mature cells produced in a specified time or an increase in the amount of maturation-inducing cytokine production that is higher than what is normally observed.

Methods of Assaying an Immune Response

In some embodiments, the invention includes assay of an immune response, for example, the ability of a compound to modulate (increase or decrease) an immune response. The assayed immune response can be an in vitro or an in vivo response.

Methods of assaying an immune response in vitro include, without limitation: up regulation of maturation markers, CD-80, CD-86, MHC-I and MHC-II, CCR-7, e.g., using FACS or RT-PCR; mixed lymphocyte/leukocyte reaction (MLR), which measures the ability of a dendritic cell to stimulate a T cell by thymidine incorporation; transwell migration assay to measure migration towards a chemokine gradient, which mimics in vivo migration to the regional lymph nodes; and FITC-dextran or ovalbumin antigen uptake and processing.

Methods of assaying an immune response in vivo include, without limitation, e.g., migration of dendritic cells, activation of T cells to the regional lymph nodes upon antigen challenge, antigen clearance, for example, if an animal is infected with Listeria monocytogenes, the speed with which the animal is able to clear Listeria from the spleen and liver is an indication of how effective the immune response is. Listeria clearance is dependent on macrophages/DCs, but also dependent on T cells. Other nonlimiting methods of assaying an immune response include measuring serum cytokine levels of an infected animal, measurement of helper and cytotoxic T cell responses, and activation of T cells in an animal model.

Cbl-b Assays

The invention also relates to methods of identifying compounds that modulate HPK1 via modulation of Casitas lineage lymphoma b (Cbl-b) expression or activity. In general, a cell is contacted with a test compound, and the ability of the compound to modulate Cbl-1 expression or activity is assayed. A compound that decreases Cbl-1 expression or activity is a candidate for decreasing HPK1 expression or activity. Accordingly, such a compound is also a candidate compound for enhancing an immune systems response such as enhancing migration of dendritic cells. A compound that increases Cbl-b expression or activity is a candidate compound for increasing HPK1 expression or activity. Such compounds are also useful for inhibiting an immune response. Compounds that modulate Cbl-b expression or activity and modulate HPK1 expression or activity are useful for treating a subject having a disorder for which it is desirable to modulate an immune system response. “Subject”, as used herein, refers to a mammal, e.g., a human, or to an experimental or animal or disease model. The subject can also be a non-human animal, e.g., a mouse, rat, guinea pig, horse, cow, goat, or other domestic animal.

Cbl-b expression can be determined by assaying expression of Cbl-b protein or RNA. Methods known in the art can be used for such determinations, including, without limitation, Western blot assay, Northern blot assay, quantitative PCR, immunoassay methods, and mass spectroscopy. Cbl-b sequences are known in the art (e.g., GenBank® (NCBI) Accession No. NM 008279 (murine HPK1), NP 009112 (human HPK1). Antibodies that are useful for immunoassay methods of detecting Cbl-b have been described (Soubeyran et al., 2002, Nature 416:183-187) and anti-Cbl-b are commercially available (e.g., Santa Cruz Biotechnology, Santa Cruz, Calif.).

Cbl-b has multiple activities. In general, certain methods of the invention relate to identifying compounds that do not modulate the ubiquitin ligase activity of Cbl-b, e.g., compounds that modulate HPK1 activity and do not modulate Cbl-b ubiquitin ligase activity. Signals that activate HPK1 are associated with the C-terminal portion of Cbl, in particular the tyrosine residues that forms the Crk binding site. Compounds that bind to the SH2 domain of Crk or Crk-L, such as a phosphorylated peptide that corresponds to at least one of the two Crk binding sites on Cbl-b are useful in the invention. Blocking the interaction between Cbl-b and Crk family members will block Cbl-b-mediated HPK1 activation. Accordingly, a screen for compounds that effect this block are performed using, e.g., a surface plasmon resonance (SPR) method (such as a BIACore system, Uppsala, Sweden), which takes advantage of this SPR property. An assay is developed in which a protein:protein interaction system containing the SH2 domain of Crk or Crk-L is fixed onto the surface of flow cells and the tyrosine phosphorylated peptide corresponding to the phosphorylated tyrosine reside of Cbl-b is passed through the flow cells. If the peptide binds to the SH2 domain, the plasmon resonance index is altered. Thus addition of inhibitor that disrupts the binding alters the plasmon resonance index, revealing the inhibition of Crk:Cbl-b has occurred. Such compounds are candidate compounds for modulating HPK1 activity.

Animal Models

According to this aspect of the invention, compounds are tested for their ability to modulate HPK1 activity and thereby to modulate a T cell response or other immune system response such as modulation of dendritic cell migration. In such methods, a subject, e.g., a mammal, such as a mouse, rat, dog, cat, guinea pig, or non-human primate, is administered a test compound. In general, the test compound is a compound that has been identified as a compound that modulates HPK1 activity. The test compound is administered to the subject and the subject is monitored at appropriate times for modulation of at least one selected immune system function. For example, in a mouse, dendritic cell migration is monitored at intervals for about 24 hours after administration of the test compound. Sampling times are adjusted as appropriate for the species of the subject. The rate of migration is compared to the rate of migration in a matched subject or pool of subjects that were not administered the test compound. A change in the rate of migration indicates that the test compound is a modulator of HPK1-mediated modulation of an immune response. For example, a compound that decreases HPK1 activity increases the rate of dendritic cell migration in the subject.

Other indicators of HPK1 inhibition include splenomegaly, which can be assayed by palpitating the subject or measurement of the spleen, e.g., at one month, three months, six months, or longer. Another indicator of HPK1 inhibition that can be assayed is hyperproliferation of hematopoietic cells, e.g., T cells or B cells. This is generally assayed after one month, three months, six months, or longer. HPK1 inhibition can also be detected by assaying resistance to PGE₂-induced immune suppression. For example, primary peripheral T cells are purified from lymph nodes and spleens. Cells from wild type and HPK1^{-/-} mice are activated by ConA or by antibody-mediated TCR crosslinking in the presence or absence of PGE₂. Wild type T cells are expected to make less IL-2 and proliferate more slowly than the wild type in the presence of PGE₂. HPK1^{-/-} cells are expected to be resistant to PGE₂ suppression. Other useful methods include assaying augmented dendritic cell function. For example, immature dendritic cells are induced to undergo maturation by LPS. Matured DC are then analyzed by FACS for increased activation marker. This is followed by functional read out where varying amounts of DC from one MHC haplotype can be use to induce proliferation by responding T cells of different haplotypes in a “mixed lymphocyte reaction” assay. An increase in T cell proliferation (measured by the amount of incorporated ³H-thymidine after an 18 hour-pulse at 72 hours or 96 hours after DC are introduced into T cell cultures, indicates that blocking HPK1 activity renders DC more effective in its antigen presentation function. It is also useful to assay increased stimulation of T cells by assaying an increase in T cell activation markers such as CD69 or CD25; or measuring cytokine production (e.g., IL-2 levels) or cellular proliferation, as described herein. Augmented dendritic cell function can also be assayed using other methods known in the art such as by detecting priming/activation of T cells, migration of cells to regional lymph nodes, secretion of IL-12, secretion of IL-6, or secretion of TNF-alpha.

Another indicator of an immune response that can be assayed is PGE₂-induced immune suppression. A compound that inhibits this response is a candidate compound for inhibition of HPK1 activity. One method of assaying PGE₂ induction of immune suppression is assaying IL-2 production. Sustained release of IL-2 in the presence of PGE₂ and a test compound indicates that the compound inhibits PGE₂ induction of an immune response. IL-2 release is assayed using methods known in the art. Other methods of determining PGE₂ immune suppression include proliferation assays and cytokine release assays that are used to measuring immunosuppressive effects of PGE₂ (Walker et al., 1983, J. Immunol. 130:1770).

Examples of animal models useful, e.g., for testing a compound that modulates HPK1 expression or activity include an autoimmune encephalomyelitis EAE model of multiple sclerosis (reviewed in Martin et al., 1992, Ann. Rev. Immunol. 10:153). In this model, myelin oligodendrocyte glycoprotein (MOG) is injected into mice to induce CNS demyelination. It is generally accepted that EAE is caused by over-active CD4⁺ T cells producing excess pro-inflammatory cytokines. The loss of HPK1 leads to the increase in pro-inflammatory cytokines. MOG-treated mice can be treated with a compound that is a candidate for inhibiting HPK1 expression or activity and tested for the expression or activity of pro-inflammatory cytokines. An increase in one or much of the cytokines indicates that the candidate compound is effective in vivo for decreasing HPK1 activity.

Another example of a useful model is collagen-induced arthritis (CIA) in a mouse model of autoimmune polyarthritis, sharing similarities with rheumatoid arthritis (RA) (reviewed in Brand et al., 2003, Springer Semin. Immunopathol. 25:3). Mice are induced to develop CIA by immunizing them with collagen type II to produce auto-reactive B cells. Auto-reactive B cells secreting antibodies against collagen type II is the primary cause of the RA-like symptoms in this animal model. Thus, a compound that increases HPK1 activity is useful for reducing the production of auto-reactive antibodies by B cells. Compounds that are candidates for increasing HPK1 expression or activity can be administered to CIA mice and the mice monitored for physical symptoms of arthritis or biochemical markers such as the production of auto-reactive antibodies by B cells. A compound that ameliorates symptoms or, for example, reduces the production of auto-reactive antibodies is useful for treating arthritis.

Other useful animal models are those for cancers, for example, a BCR-Abl p210 transgenic model of leukemia. Expression of a BCR-Abl p210 transgene causes spontaneous expansion of hematopoietic cells that develop subsequently into leukemia (Honda, 1995, Rinsho Ketsueki 36:559). Even though c-Abl has been shown to be an activator of HPK1 kinase activity, as a response to disregulated Abl kinase activity (such as that found in BCR-Abl), HPK1 activation is merely a cellular response to BCR-Abl transformation (Ito et al., 2001, J Biol. Chem. 276:18130). Thus, an elevated expression of HPK1 can be useful for delaying or preventing BCR-Abl-induced transformation. Compounds that are candidates for increasing HPK1 expression or activity can be tested for their ability to ameliorate physical symptoms or biochemical markers of leukemia can be useful for treating such disorders.

In some cases, it is beneficial to decrease HPK1 activity. Animal models for cancers of this type include, without limitation, a PGE₂-producing tumor model. For example, murine Lewis lung carcinoma is a model that has been used to study both metastatic and local tumors. Variant lines of 3LL, a cell line originally isolated and cloned from metastatic lung nodules of C57BL/6 mice, differ in tumorigenecity or/and metastatic lung disease. In the metastatic model, mice become moribund or die within 4 to 5 weeks after intravenous injection of tumor cells and the time of death and number of lung nodules can be quantified. A less aggressive tumor cell line forms local solid tumors at the site of subcutaneous (sc) inoculation that can be assessed for tumor size and mass. Increased tumor growth and migration has been attributed to the response of the tumor to the production of PGE₂ (Young et al., 1991, Int. J. Cancer 49:150; Teicher et al., 1994, Cancer Chemother. Pharmacol. 33:515). Investigators have established a more direct link between systemic levels of PGE₂ and tumor activity by using various cyclooxygenase and lipoxygenase inhibitors (Stolina et al., 2000, J. Immunol. 164:361; Levin et al., 2000, Chemother. 46:429). It has also been demonstrated that the PGE₂ receptor EP2 is important for PGE₂ mediated inhibition of DC function and anti-tumor response, as shown using EP2^{-/-} animals (Yang et al., 2003, J. Clin. Invest. 111:727). A compound that is a candidate for decreasing HPK1 expression or activity can be administered to animals engrafted with 3LL cells and monitored for physical symptoms, biochemical, and histological markers of tumor development or metastasis. Compounds that ameliorate symptoms, reduce biochemical markers of tumor development, or result in improved pathology are useful for treating such tumors.

Methods described herein for identifying a compound that modulates HPK1 expression or activity can be combined. For example, identification of a compound that may indirectly modulates HPK1 (e.g., by modulating expression or activity of PKA or Cbl-b) can be combined with determining whether the compound modulates HPK1.

Transgenic animals that do not express HPK1 or have reduced expression of HPK1 are useful, e.g., for establishing a reference. A compound that inhibits expression or activity of an HPK1 is expected to have an effect on selected features corresponding to those in an HPK1^{-/-} animal. Such animals are also useful for identifying features that are characteristic of HPK1 inhibition.

Methods of making such transgenic animals are known in the art and a non-limiting example of generating an HPK1^{-/-} knockout mouse is provided in the Examples.

Methods of Treatment

Compounds that decrease HPK1 expression or activity can be used to enhance an immune response, e.g., increase dendritic cell migration (e.g., the rate of migration or the number of cells migrating), alter cytokine production, enhance a T cell response, or a combination of these functions.

The present invention provides for both prophylactic and therapeutic methods of treating a subject at risk of (or susceptible to) a disorder or having a disorder associated with aberrant or unwanted HPK1 expression or activity. As used herein, the term “treatment” is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. A therapeutic agent includes, but is not limited to, small non-nucleic acid organic molecules, small inorganic molecules, peptides, synthetic peptides, antibodies and fragments thereof, nucleic acids (e.g., ribozymes, siRNA, and antisense oligonucleotides).

In one aspect, the invention provides a method for preventing in a subject, a disease or condition associated with an aberrant or unwanted HPK1 expression or activity, by administering to the subject an agent that modulates HPK1 expression or at least one HPK1 activity. Subjects at risk for a disease that is caused or contributed to by aberrant or unwanted HPK1 expression or activity can be identified, for example, by unwanted or aberrant activity of the immune system or by a condition in which it is desirable to enhance immune system activity. Administration of a prophylactic agent can occur prior to the manifestation of symptoms, such that a disease or disorder is prevented or, alternatively, delayed in its progression. Depending on whether it is desirable to increase or decrease HPK1 expression or activity, for example, an HPK1, HPK1 agonist or HPK1 antagonist agent, can be used for treating the subject. The appropriate agent can be determined based on screening assays described herein.

It is possible that some disorders are associated with a deficiency of HPK1 activity or an aberrant overproduction of HPK1. As such, the reduction in the level and/or activity of such gene products brings about the amelioration of disorder symptoms.

Compounds that increase HPK1 activity are useful for treating, e.g., hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Generally, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease, Hodgkin's lymphoma, and Reed-Stemberg disease. Other disorders that may be treated with compounds that increase HPK1 activity include autoimmune diseases (for example, diabetes mellitus, arthritis (including rheumatoid arthritis, juvenile rheumatoid arthritis, osteoarthritis, psoriatic arthritis), systemic lupus erythematosis, autoimmune thyroiditis, dermatitis (including atopic dermatitis and eczematous dermatitis), psoriasis, Sjögren's Syndrome, Crohn's disease, aphthous ulcer, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy such as, atopic allergy.

The identified compounds that inhibit HPK1 activity can be administered to a subject (e.g., a non-human mammal such as a dog, cat, bovine, porcine, goat, mouse, rat, or horse; or to a human) at therapeutically effective doses to prevent, treat, or ameliorate disorders in which it is desirable to increase immune system activity, e.g., to enhance T cell function. A therapeutically effective dose refers to that amount of the compound sufficient to result in amelioration of symptoms of the disorders. Toxicity and therapeutic efficacy of such compounds can be determined by pharmaceutical procedures that are known in the art, for example, by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally selected. While compounds that exhibit toxic side effects may be used, care should be taken to design a delivery system that targets such compounds to the site of affected tissue to minimize potential damage to uninfected cells and, thereby, reduce side effects.

Data obtained from the cell culture assays and animal studies can be used in formulating a range of dosage for use in humans. The dosage of such compounds generally lies within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography or mass spectroscopy.

Another example of determination of effective dose for an individual is the ability to directly assay levels of “free” and “bound” compound in the serum of the test subject. Such assays may utilize antibody mimics and/or “biosensors” that have been created through molecular imprinting techniques. The compound that is able to modulate HPK1 activity is used as a template, or “imprinting molecule”, to spatially organize polymerizable monomers prior to their polymerization with catalytic reagents. The subsequent removal of the imprinted molecule leaves a polymer matrix that contains a repeated “negative image” of the compound and is able to selectively rebind the molecule under biological assay conditions, (see, e.g., Ansell et al, 1996, Curr. Op. Biotechnol. 7:89-94, and Shea, 1994, Trends Polymer Sc. 2:166-173. Such “imprinted” affinity matrixes are amenable to ligand-binding assays, whereby the immobilized monoclonal antibody component is replaced by an appropriately imprinted matrix. An example of the use of such matrixes is found in Vlatakis et al. (1993, Nature 361:645-647). Through the use of isotope labeling, the “free” concentration of compound that modulates the expression or activity of HPK1 can be readily monitored and used in calculations of IC₅₀.

Such “imprinted” affinity matrixes can also be designed to include fluorescent groups whose photon-emitting properties measurably change upon local and selective binding of target compound. These changes can be readily assayed in real time using appropriate fiber optic devices, in turn allowing the dose in a test subject to be quickly optimized based on its individual IC₅₀. A rudimentary example of such a “biosensor” is discussed in Kriz et al. (1995, Analy. Chem. 67:2142-2144).

An aspect of the invention pertains to methods of modulating HPK1 expression or activity for therapeutic purposes. Accordingly, in an exemplary embodiment, the modulatory method of the invention involves contacting a cell with an HPK1 or agent that modulates one or more of the activities of HPK1 activity associated with the cell. An agent that modulates HPK1 protein activity is an agent as described herein, such as a nucleic acid or a protein, a naturally-occurring target molecule of a HPK1 protein (e.g., a HPK1 substrate or receptor), a HPK1 antibody, a HPK1 agonist or antagonist, a peptidomimetic of a HPK1 agonist or antagonist, or other small molecule.

In one embodiment, the agent stimulates one or more HPK1 activities. Examples of such stimulatory agents are described herein. In another embodiment, the agent inhibits one or more HPK1 activities. These modulatory methods can be performed in vitro (e.g., by culturing a cell with the agent) or, alternatively, in vivo (e.g., by administering the agent to a subject). As such, the present invention provides methods of treating an individual afflicted with a disease or disorder characterized by aberrant or unwanted expression or activity of a HPK1 protein. In one embodiment, the method involves administering an agent (e.g., an agent identified by a screening assay described herein), or combination of agents that modulate (e.g., up regulates or down regulates) HPK1 expression or activity. In another embodiment, the method involves administering a HPK1 protein or nucleic acid molecule as therapy to compensate for reduced, aberrant, or unwanted HPK1 expression or activity.

Pharmaceutical Compositions

Compounds identified using the methods described herein can be incorporated into pharmaceutical compositions. Such compounds are useful for increasing an immune response, e.g., in an immunocompromised subject, or for decreasing a response, e.g., in a subject that has an autoimmune disorder. Such compositions typically include the compound and a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include, but are not limited to, parenteral, e.g., intravenous, intradermal, subcutaneous, inhalation, transdermal (topical), transmucosal, and rectal; or oral administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition is sterile and is fluid to the extent that easy syringability exists. The compounds stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some cases, isotonic agents are included, such as, for example, sugars, polyalcohols such as manitol, sorbitol, or sodium chloride in the composition. Prolonged absorption of the injectable compositions can be accomplished by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the pharmaceutical formulations according to the invention are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical formulation can also be prepared in the form of suppositories (e.g., with conventional suppository bases such as cocoa butter and other glycerides) or retention enemas for rectal delivery.

In one embodiment, the compounds identified as described herein are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from, e.g., Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

As defined herein, a therapeutically effective amount of protein or polypeptide (i.e., an effective dosage) ranges from about 0.001 mg/kg to about 30 mg/kg body weight, about 0.01 mg/kg to about 25 mg/kg body weight, about 0.1 mg/kg to about 20 mg/kg body weight, or about 1 mg/kg to about 10 mg/kg, about 2 mg/kg to about 9 mg/kg, about 3 mg/kg to about 8 mg/kg, about 4 mg/kg to about 7 mg/kg, or about 5 mg/kg to about 6 mg/kg body weight. The protein or polypeptide can be administered one time per week for between about 1 week to about 10 weeks, between about 2 weeks to about 8 weeks, between about 3 weeks to about 7 weeks, or for about 4 weeks, about 5 weeks, or about 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

For antibodies, the dosage is generally about 0.1 mg/kg of body weight (generally about 10 mg/kg to about 20 mg/kg). If the antibody is to act in the brain, a dosage of about 50 mg/kg to about 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. (1997, J Acquired Immune Deficiency Syndromes and Human Retrovirology 14:193).

The present invention encompasses agents that modulate expression or activity of an HPK1 gene or protein. An agent may be a small molecule, for example. Such small molecules include, but are not limited to, peptides, peptidomimetics (e.g., peptoids), amino acids, amino acid analogs, polynucleotides, polynucleotide analogs, nucleotides, nucleotide analogs, non-nucleic acid organic compounds or inorganic compounds (including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram (μg) per kilogram (kg) to about 500 milligrams (mg) per kg, about 100 mg per kg to about 5 mg per kg, or about 1 mg per kg to about 50 mg per kg. It is furthermore understood that appropriate doses of a small molecule depend upon the potency of the small molecule with respect to the expression or activity to be modulated. When one or more of these small molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A nucleic acid sequence encoding a molecule of the invention (e.g., a sequence encoding an siRNA that can modulate HPK1 activity) can be inserted into a vector and used as a gene therapy vector. Gene therapy vectors can be delivered to a subject by, for example, intravenous injection, local administration (see e.g., U.S. Pat. No. 5,328,470) or by stereotactic injection (see e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Compounds of the invention are used in the preparation of a medicament for treating an HPK1-related disorder, e.g., by enhancing an immune response in an immunosuppressed individual.

### EXAMPLES

The invention is further illustrated by the following examples. The examples are provided for illustrative purposes only. They are not to be construed as limiting the scope or content of the invention in any way.

### Example 1

### Role of HPK1 in Dendritic Cell Migration and Maturation

Materials

Rabbit anti-murine HPK1 polyclonal antibodies # 5 and #6 were used as the immunoprecipitating antibodies, and the rabbit anti-murine HPK1 antibody #7 was used for detecting HPK1 in Western blot (Tibbles et al., 1996, EMBO J. 15:7013-7025).

Horseradish peroxidase-conjugated (HRP-conjugated) anti-rabbit polyclonal antibody was from Amersham Biosciences (Piscataway, N.J.). The following antibodies were purchased from Pharmingen BD Biosciences (San Diego, Calif.): Fluorescein isothiocyanate (FITC)-conjugated hamster anti-mouse CD80, r-phycoerythrin (PE)-conjugated I-A^{b}, PE-conjugated CD11c and (PE)-conjugated rat anti-mouse CD86, and mouse anti-mouse I-A^{b}. RPMI 1640 (Cellgro, Va.) supplemented with 10% fetal bovine serum (FBS; Gemini BioProducts, Woodland, Calif.), 2-β-mercaptoethanol (2-ME, 50 μM) from Invitrogen/Gibco® (Carlsbad, Calif.), and L-glutamine (2 mM)/penicillin (100 U/ml)/streptomycin (100 μg/ml) from Gemini Bio-Products (Woodland, Calif.) was used as a complete dendritic cell medium. Mouse CCL-21 and recombinant murine granulocyte and macrophage colony stimulating factor (rmGM-CSF) were from R&D systems (Minneapolis, Minn.). Salmonella and E. coli lipopolysaccharide (LPS), concanavalin A (ConA; Canavalia ensiformis) and 5(6)-carboxyfluorescein diacetate N-succinimidyl ester (CFSE) were from Sigma-Aldrich (St. Louis, Mo.). Protein A Sepharose™ beads were from Amersham Biosciences (Piscataway, N.J.).

Dendritic Cell Preparation

Bone marrow derived dendritic cells (BMDCs) were prepared as described in Lutz et al., (1999, J. Immunol. Meth. 223:77-92). The femur and tibia were removed from a mouse and the bone marrow was flushed out using a 25-gauge needle. The cells were then grown in complete dendritic cell medium (supra) plus 20 ng/ml mrGM-CSF for 10 days in Petri dishes (2×10⁶ cells/dish). Non-adherent cells were then transferred to tissue culture dishes and left as is (immature dendritic cells), or matured in the presence of LPS or other maturation stimuli.

Immunoprecipitation and Western Blot Analysis

Cells were lysed in lysis buffer consisting of Tris, pH 7.6, 150 mM NaCl, 1 mM sodium orthovanadate, 10 mM sodium fluoride, 10 mM sodium pyrophosphate, 2 mM phenylmethylsulphonyl fluoride (PMSF), 1% NP-40, and a cocktail of protease inhibitors (Protease Inhibitor Cocktail Set I obtained from EMD Biosciences, Inc., La Jolla, Calif.) Lysates were then immunoprecipitated by incubation with 1 μl of polyclonal HPK1 antibodies #5 and #6 at and protein A beads at 4° C. overnight. The samples were then washed three times in a wash buffer containing 0.75 M NaCl, 1 M HEPES, pH 7.4, glycerol, sodium orthovanadate, 0.5 M NaF, 1% NP-40, and protease inhibitors, and then boiled in 1× loading buffer for 3 min. Samples were separated over an 8% polyacrylamide gel, transferred to a PVDF membrane, probed with anti-murine HPK1 #7 and then detected with HRP-conjugated anti-rabbit antibodies. Immunodecorated proteins were visualized with an enhanced chemiluminescence immunoblotting detection system (PerkinElmer, Wellesley, Mass.).

FACS Analysis

Immature and LPS matured dendritic cells were resuspended in labeling medium (5% FBS, 0.1% NaN3 in PBS) and then 10⁶ cells were incubated with the appropriate antibodies conjugated to FITC or PE for 50 min. at 4° C. The cells were then washed twice with labeling medium, the pellet was resuspended in 1×PBS, and then was analyzed using flow cytometry in a fluorescently activated cell sorter (FACS). Flow cytometry was done on a FACSCalibur™ (BD Biosciences, San Jose, Calif.) and analyzed with CellQuest™ software (BD Biosciences).

Mixed Leukocyte Reaction

The mixed leukocyte reaction (MLR) was performed as described in Salusto et al. (1994, J. Exp. Med. 179:1109-1118), with modifications. Briefly, naïve T cells from an allogeneic SJL mouse spleen were purified using CD62L magnetic beads (Miltenyi Biotech Inc., Auburn, Calif.). In a 96-well round bottom plate, purified naïve T cells (2×10⁵) were plated for use as responders and incubated with varying dilutions of HPK1^{-/-} and wild type, mature and immature, BMDCs as stimulators. Cells were pulsed with 1 μCi ³H-thymidine for 20 hours before harvesting. Naïve T cells treated with 10 ng ConA were used as a positive control.

Migration Assay

Assays were performed using Transwell® migration chambers (Costar, Corning, N.Y.). Transwell migration assays were carried out using Transwell® migration chambers with 5.0 M pore size inserts. Immature dendritic cells (2×10⁴) and LPS matured dendritic cells were resuspended in 1.5 ml of complete dendritic cell medium and added to the top of a migration chamber. The bottom chamber was filled with either 1.5 ml of medium alone, or medium plus 30 ng/ml CCL-21. Plates were incubated for 45 min., 90 min., or 180 min. at 37° C. with 5% CO₂. After the indicated time, cells collecting in the bottom chamber were centrifuged at 6000×g, and the number of migrated cells was counted.

In Vivo Migration

Immature bone marrow derived dendritic cells (6×10⁶) from HPK1^{+/+} (+/+) and HPK1^{-/-} (−/−) were labeled with carboxy-fluorescein diacetate succinimidyl ester (CFSE), washed, and injected along with 25 ng of E. coli LPS into the footpad of wild type C57B1/6 mice. After 12 or 24 hours, both the popliteal and inguinal lymph nodes are removed, and number of cell migrated into the nodes was examined using FACSCalibur™.

HPK1 Expression in Immature Dendritic Cells

To assess the expression of HPK1 in dendritic cells, bone marrow cells were cultured in the presence of GM-CSF (20 ng/ml) for 10 days to yield immature BMDCs. BMDCs were left in the immature state, or matured with 2 μg/ml E. coli lipopolysaccharide (LPS) or with Salmonella LPS for 24 hours. Cells were then lysed and immunoprecipitated with an anti-HPK1 antibody. Immunoprecipitates were Western blotted with anti-HPK1 antibody followed by HRP-conjugated anti-mouse Ig secondary antibody.

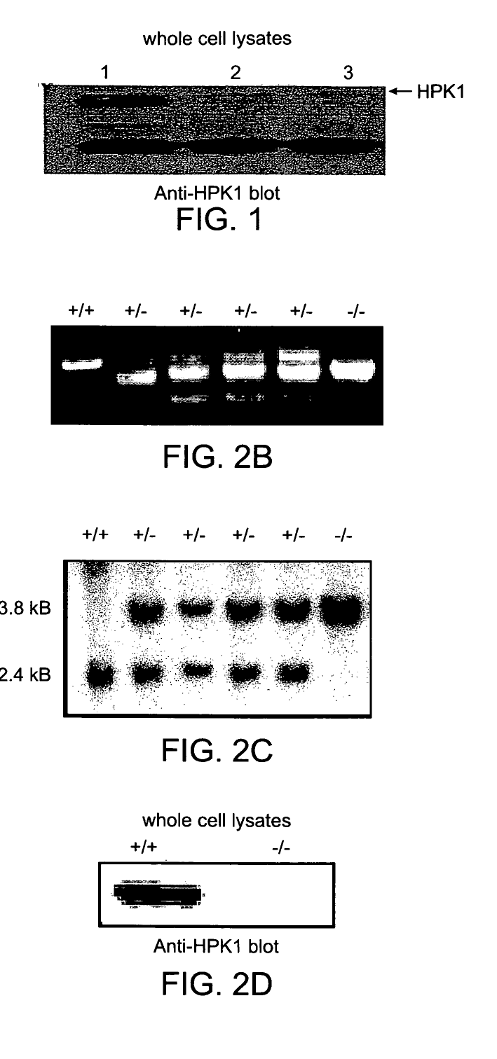

HPK1 was expressed in immature dendritic cells and HPK1 expression was markedly diminished upon incubation with LPS (FIG. 1). The disappearance of HPK1 upon exposure to a maturation stimulus contrasts with previous reports where the expression of HPK1 in other cell lineages is either unchanged, or is increased upon cell surface receptor antigen engagement.

HPK1^{-/-} Mice

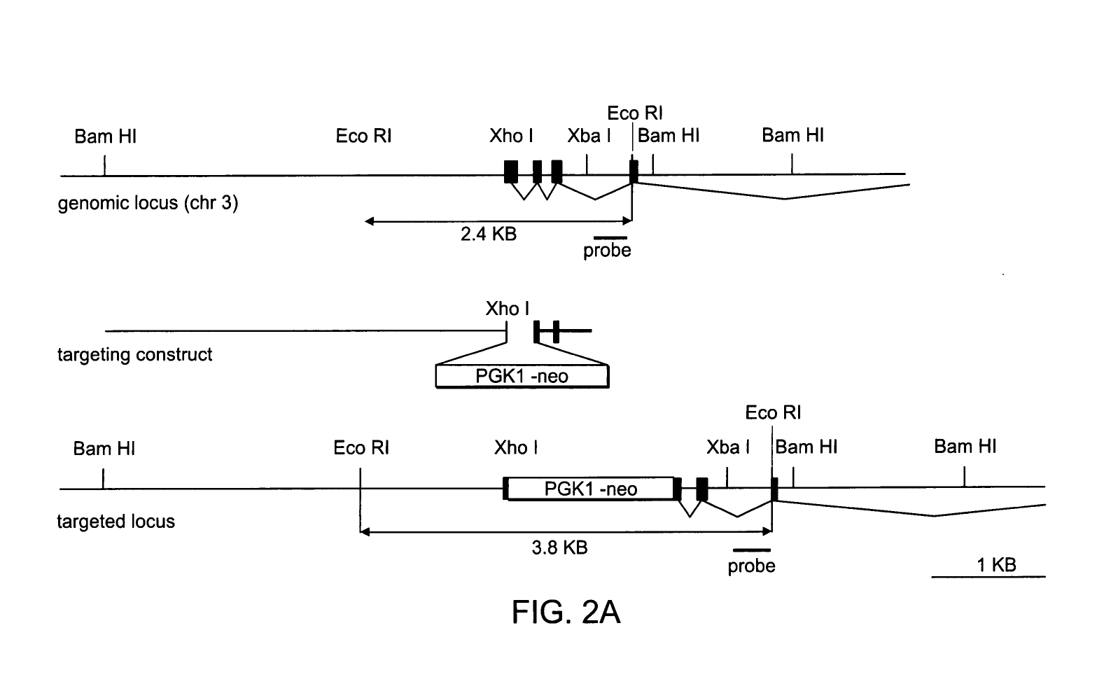

To more specifically define the role of HPK1 in dendritic cell function, studies were performed using mice that lack functional HPK1. HPK1^{-/-} mice that were functionally deleted in a portion of the first exon of the Hpk1 gene. The mice were generated using methods known in the art. FIG. 2A is a diagram illustrating the targeting strategy. Briefly, a 4.7 kbp genomic BamHI-XhoI fragment covering the first 3 exons of HPK1 was inserted into a pMC1-neo vector, which served as the long arm of the targeting construct. The second half of exon 1 and the adjacent intron were replaced with a PGK1-neo selection cassette in antisense orientation. A 466 bp fragment starting with exon 2 was generated by PCR amplification and served as the short arm of the targeting construct. The linearized construct was electroporated into E14 embryonic stem cells. Positive clones were identified by PCR with the primers: 5′-GGG AGC CAA GAA ATT TGA GAG CTC-3′ (common primer; SEQ ID NO:2), 5′-CCG GTG GAT GTG GAA TGT GTG-3′ (targeted allele; SEQ ID NO:3) and CCC TTC TGT CTC CTC CAC CAC (wild type allele; SEQ ID NO:4) and injected into C57BL/6 blastocysts. This resulted in a null mutation of the HPK1 locus as confirmed by PCR (FIG. 2B). Tail genomic DNA was subjected to PCR analysis using primer pairs specific for the wild type HPK1 allele and neo-specific primer. A 726 nucleotide fragment was expected for the wild type allele and a 670 nucleotide fragment was expected for the HPK1-disrupted allele. Southern blot analysis was performed on genomic DNA was digested with Eco RI and hybridized with the 3′ flanking probe. FIG. 2C illustrates the 2.4 kb fragment is expected for the wild type allele and a 3.8 kb fragment would indicate the presence of the neomycin cassette. These data demonstrate the deletion using a 3′-external probe. The wild type chromosomal locus gives rise to a 2.4 kb fragment, while a 3.8 kb fragment is generated from the targeted locus. Western blot analysis was performed to confirm the lack of HPK1 protein in the knockout mice (FIG. 2D). These mice, termed “Hpk1^{-/-}”, appeared to be were healthy, normal, reproduced with Mendelian ratios, and were somewhat less fertile compared to wild type.

It appears that a truncated HPK1 can bind to other signaling proteins and sequester those proteins from their normal binding partner, thereby functioning as a dominant negative molecule.

Expression of Maturation Markers in HPK1^{-/-} BMDCs

To assess the role of HPK1 in dendritic cell function, the generation of immature HPK1^{-/-} BMDCs was analyzed, and whether the ability of these cells to mature in response to LPS stimulation was affected. The generation of immature HPK1^{-/-} dendritic cells from bone marrow was analyzed by assaying the expression of a dendritic cell surface marker, CD11c. Subsequently, the development of immature dendritic cells to mature dendritic cells was assessed by the ability of dendritic cells to up-regulate known maturation markers.

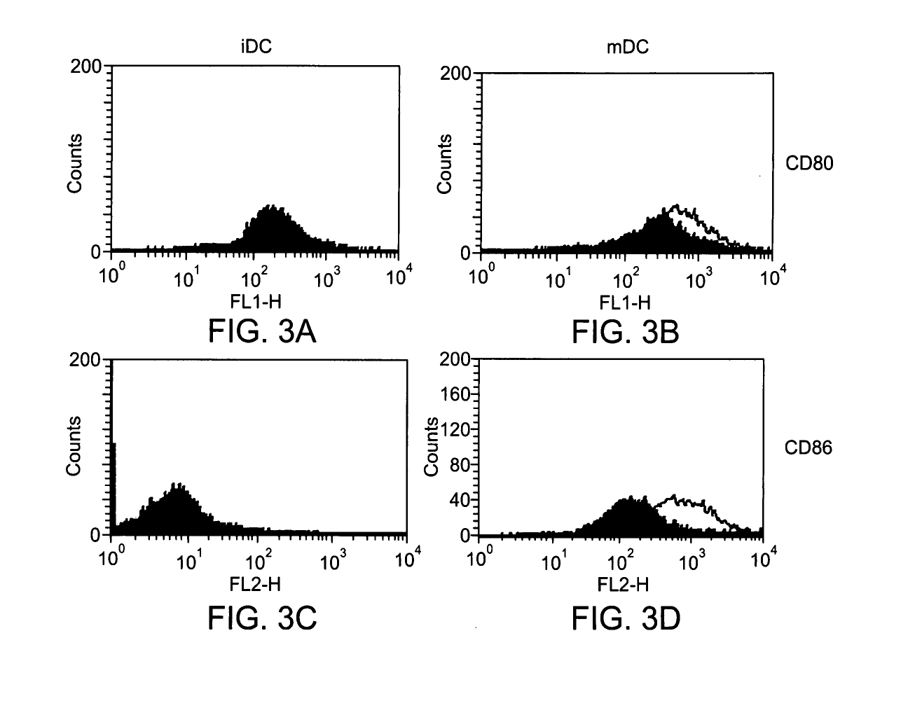

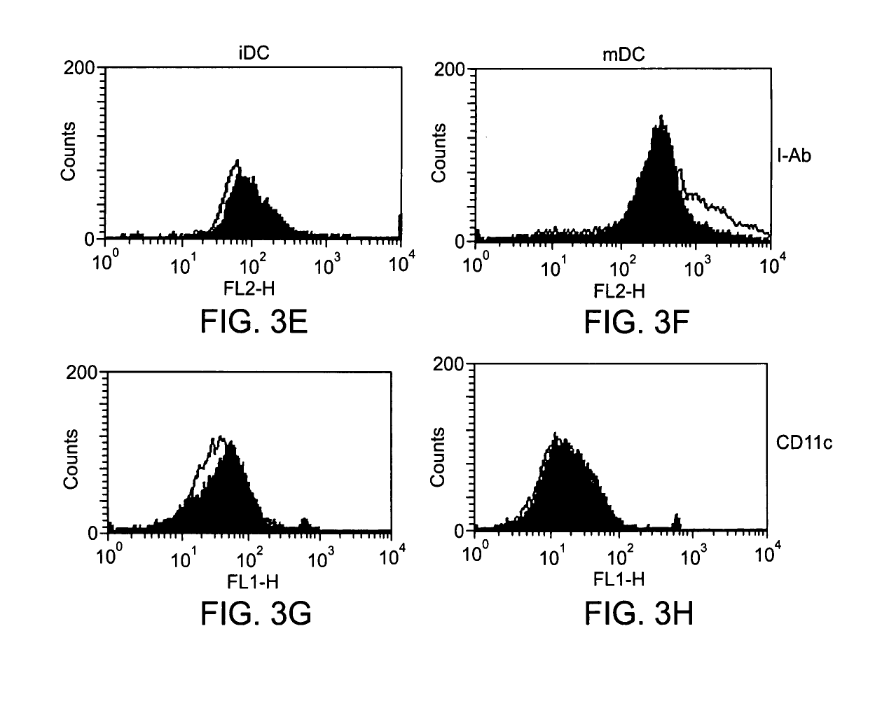

Briefly, bone marrow cells were cultured for ten days in complete dendritic cell medium with 20 ng/ml GM-CSF. The cells were then stained with FITC-conjugated anti-CD11c antibody and the level of FITC observed. There was no apparent difference in the level of CD11c on the surface of wild type and HPK1^{-/-} dendritic cells (FIGS. 3G and 3H). These results suggest HPK1 does not play a role in the development of immature dendritic cells.

To determine whether HPK1 plays a role in dendritic cell maturation, immature or LPS-matured BMDCs were stained with FITC-conjugated anti-CD80, as well as with PE-conjugated anti-CD86 and anti-1-A^{b} antibodies. These are antibodies that recognize known maturation markers involved in T cell signaling and antigen presentation. After 24 hours of LPS maturation, wild type BMDCs had up regulated maturation markers. In contrast, dendritic cells from the HPK1^{-/-} mice displayed a higher mean florescence intensity in the surface expression of CD80, CD86, and I-A^{b} (FIG. 3A-FIG. 3H). These data suggested that HPK1 is either directly or indirectly involved in the negative regulation of LPS-induced up-regulation of dendritic cell maturation markers. Therefore, HPK1 may prevent dendritic cells from ‘fully’ responding to LPS. Accordingly, induction of HPK1 activity can be a means of decreasing dendritic cell maturation. Conversely, decreasing HPK1 activity is a means of increasing dendritic cell maturation.

HPK1^{-/-} Bone Marrow-Derived Dendritic Cells as Improved T Cell Stimulators

Since LPS-matured BMDCs from the HPK1^{-/-} mice exhibited a higher expression of maturation markers on their cell surface, whether these cells are also functionally superior to wild type dendritic cells in activating T cells was investigated. Higher co-stimulatory molecule expression in the HPK1^{-/-} BMDCs suggested that those cells may be more proficient in signaling to T cells.

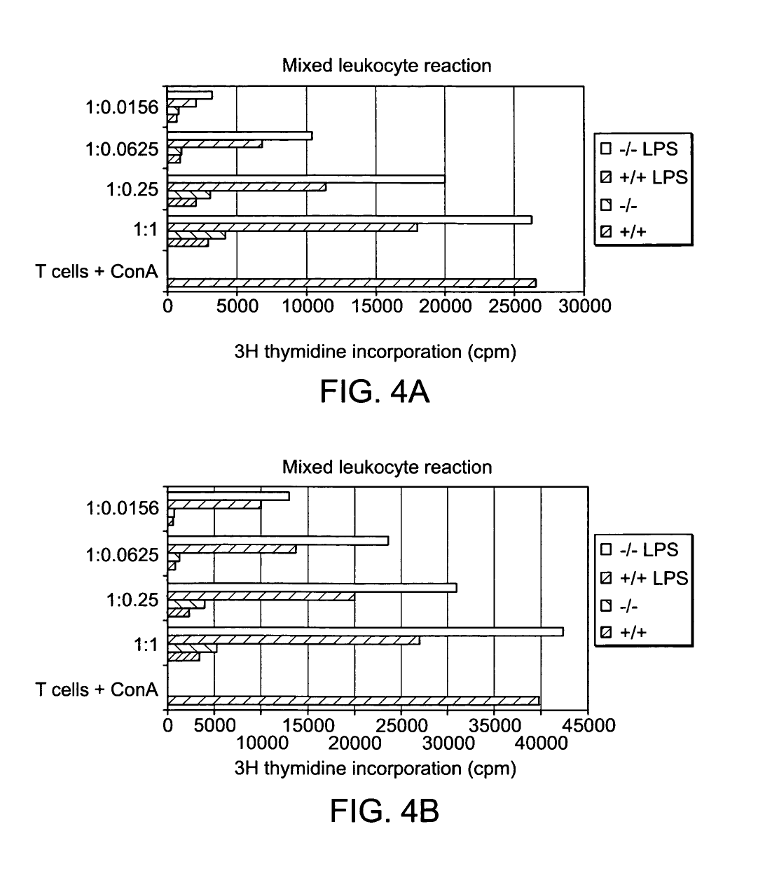

To determine the effect that HPK1 exerts on the ability of dendritic cells to stimulate T cells, immature or LPS-matured BMDCs from HPK1^{-/-} and wild type mice were co-cultured in various ratios with naïve T cells from the spleens of allogeneic mice. After 2 days of a mixed leukocyte reaction (MLR), wells containing mature HPK1^{-/-} dendritic cells already displayed greater stimulation than wild type dendritic cells (FIG. 4A). In fact, after two days of a mixed leukocyte reaction, T cell stimulation by the HPK1^{-/-} dendritic cells had already reached the maximal stimulation reached on day 4 by wild type dendritic cells (FIG. 4B). After 4 days, T cell stimulation by HPK1^{-/-} dendritic cells was nearly two-fold greater than simulation by wild type dendritic cells (FIG. 4B). The number of HPK1^{-/-} dendritic cells needed to stimulate a fixed number of T cells was assessed and was found to be four-fold less than the number of wild type dendritic cells needed to activate the same number of T cells (compare T: dendritic cell 1:0.0625 vs. 1:0.25) (FIG. 4A). These data indicate that dendritic cells lacking HPK1 not only up-regulate maturation markers better than dendritic cells that express functional HPK1, but that they are able to stimulate T cells more efficiently than wild type dendritic cells. Thus, decreasing HPK1 activity provides a method of increasing the efficiency of T cell stimulation, and thereby provides a method of enhancing an immune response or providing a general increase in immune system activity.

Chemotactic Migration of HPK1^{-/-} BMDCs

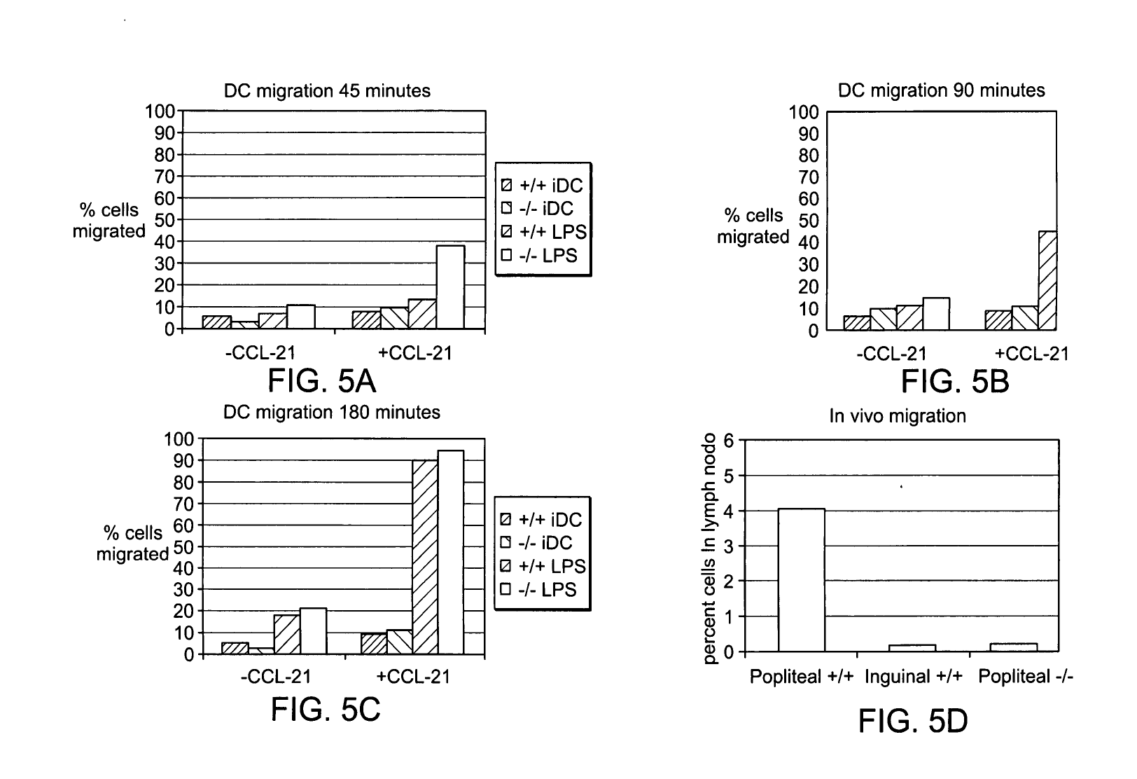

One well-accepted feature of mature dendritic cells is their ability to migrate to the regional lymph nodes and activate adaptive immune responses. The Transwell® migration assay was used as an in vitro measurement of this migratory ability. To determine the migration qualities of dendritic cells from HPK1^{-/-} mice, immature and LPS-matured HPK1^{-/-} and wild type dendritic cells were seeded on the top of a Transwell® chamber under conditions in which the bottom chamber contained medium alone or contained medium plus CCL-21. CCL-21 is expressed in peripheral lymph nodes and is the natural ligand for CCR7 on the surface of mature dendritic cells. Within 45 min., approximately 40% of the HPK1^{-/-} dendritic cells had migrated to the lower chamber, compared to fewer than 15% of the wild type cells (FIG. 5A). By 90 min., nearly 85% of HPK1^{-/-} cells had migrated to the lower chamber in contrast to 45% of the wild type dendritic cells (FIG. 5B). By 180 min., comparable levels of HPK1^{-/-} and wild type dendritic cell had migrated to the lower chamber (FIG. 5C). These results suggested that HPK1 can negatively regulate the ability of dendritic cells to migrate towards a CCL-21 chemotactic gradient. Therefore, a compound that decreases HPK1 expression or activity is useful for increasing dendritic cell migration to lymph nodes.

HPK1^{-/-} BMDC Migration to Regional Lymph Nodes

To confirm whether the in vitro pattern of migration of the HPK1^{-/-} BMDCs correlates with in vivo migration, migration efficiency was assayed in mice that were injected with CFSE labeled BMDCs. The migration assay permits tracking of the number of CFSE labeled dendritic cells that migrate from the site of injection to the regional lymph nodes after activation by LPS. In these experiments, immature dendritic cells were injected into the footpad of normal syngeneic recipient mice in the presence of LPS. After 24 hours, inguinal lymph nodes were removed from the mice, and the cells were analyzed by FACS.

It was found that CFSE-positive cells were only present in the inguinal nodes of mice injected with wild type dendritic cells but were not seen when HPK1^{-/-} dendritic cells were injected. Since the results in vitro chemotaxis assays suggest that the HPK1^{-/-} dendritic cells migrate more rapidly than wild type dendritic cells, inguinal and popliteal lymph nodes were examined 12 hours after injection. At 12 hours, when mice were injected with wild type dendritic cells, inguinal nodes had less than 0.5% CFSE positive cells and the popliteal nodes contained about 4% CFSE positive cells (FIG. 5D). These results suggest that by 12 hours wild type dendritic cells injected into the footpad have not yet reached inguinal nodes in any substantial numbers. However, in mice injected with HPK1^{-/-} dendritic cells, the popliteal nodes had few CFSE positive cells but the inguinal node had 5% CFSE positive dendritic cells (FIG. 5D). These data suggest that the HPK1^{-/-} dendritic cells migrated more quickly than their wild type counterparts, which is consistent with the in vitro migration assay.

### Example 2

### Mechanism of Prostaglandin E2 Activation of HPK1

In these tests, the ability of prostaglandin E2 (PGE₂) to activate the catalytic activity of HPK1 was studied to determine whether modulation of PGE₂ can be used to affect HPK1 activity. PGE₂ is an eicosanoid product of aracadonic acid metabolism that has immunosuppressive activity.

Reagents

Horseradish peroxidase (HRP)-coupled anti-phosphotyrosine antibody (RC20H) was purchased from Transduction Laboratories, Lexington, Ky. Anti-human HPK1 rabbit polyclonal antibody #47 was raised using methods known in the art (Sawasdikosol, et al., 2003, Blood 101:3687-3689) was used for immunoprecipitation and immunoblot assays of endogenous HPK1. Immunoprecipitation and Western blotting of ectopically expressed, hemagglutinin (HA)-tagged, murine HPK1 were performed with the 12CA5 anti-HA monoclonal antibody (mAb) and anti-murine HPK1 rabbit polyclonal antibody #7 Kiefer (1996 #805), respectively. Both the 12CA5 mAb and the anti-human CD3ε (OKT3) mAb used in TCR crosslinking experiments were purified from hybridoma supernatants in the laboratory. Rabbit anti-phospho-PKA antibody was obtained from CellSignaling, Lake Placid, N.Y. γ-[³²P]-ATP was obtained from Perkin Elmer Life Science (Boston, Mass.). Histone H2A, the exogenous substrate used in in vitro kinase reactions, was purchased from Roche Applied Science, Indianapolis, Ind., and PGE₂ was purchased from Calbiochem-Novabiochem, San Diego, Calif.

Molecular Constructs

The pcDNA3 vector containing mouse cDNA encoding the HA-tagged wild type murine HPK1 and a proline-rich-deleted construct were constructed by Dr. F. Kiefer (Liu et al., 2000, J. Immunol. 165:1417). QuickChange™ mutagenesis system was used to alter the wild type HPK1 construct to encode the desired point mutations (Stratagene, La Jolla, Calif.). Mutated constructs were sequenced to verify the presence of the desired mutation and for the absence of PCR-generated mutations.

Cells and Stimulations

JE6.1 Jurkat cells, obtained from the American Type Culture Collection (ATCC; Rockville, Md.) and its mutants were grown in RPMI 1640 complete medium (RPMI 1640 supplemented with 10% fetal bovine serum, 2 mM L-glutamine and 100 units of penicillin/streptomycin). Lck-deficient JCaM 1.6 cells and the ZAP-70-deficient p116 Jurkat cells were obtained from the American Type Culture Collection (ATCC, Manassas, Va.). The Lat-deficient Jurkat cell line was used (Zhang et al., 1999, Int. Immunol. 11:943) and the SLP-76 deficient J14 Jurkat was also used (Yablonski et al., 1998, Science 281:413). Human embryonic kidney cell lines (HEK) stably transfected with either EP2 or EP4 receptor were obtained and are described in (Desai et al., 2000, Mol. Pharmacol. 58:1279).

Stimulation of cells by the anti-CD3-antibody-mediated T cell receptor (TCR) crosslinking in suspension, cell lines (Jurkat or mutant Jurkat lines) were suspended in complete RPMI-1640 media (1×10⁷ cells/immunoprecipitation) and incubated with 1 μg of anti-human CD3ε (OKT3.14) at 4° C. as described previously (Pratt et al., 2000, J. Immunol. 165:4158). After incubation for 10 min. on ice with the stimulating antibodies, 7.5 μg of rabbit anti-mouse antibody was added for effecting crosslinking, and the samples were incubated for an additional ten min. at 4° C. The cells were then warmed to 37° C. for the indicated times.

Immunoprecipitations and Immunoblotting

Cells were lysed in a buffer containing 1% Nonidet P-40 (NP-40) and 50 mM Tris, pH 7.6, 150 mM NaCl, 1 mM Na₃V₄O₇, 10 mM NaF, 10 mM sodium pyrophosphate, 10 μg/ml each of aprotinin and leupeptin, and 2 mM phenylmethylsulfonyl fluoride (PMSF). The lysates were pre-cleared with protein A-Sepharose™ and subsequently, proteins were immunoprecipitated with either 2 μg of anti-Crk antibody or an equal amount of normal rabbit Ig by incubation at 4° C. for two hours. The beads were washed with 0.1% NP-40 in immunoprecipitation wash buffer (150 mM NaCl, 20 mM HEPES, pH 7.4, 10% glycerol, 1 mM Na₃V₄O₇, 5 mM NaF, and 10 μg/ml each of aprotinin and leupeptin), and the bead-bound proteins were separated by SDS-PAGE. The proteins were transferred to a polyvinylidene fluoride (PVDF) membrane, immunoblotted with the indicated antibodies and developed by the enhanced chemiluminescence (ECL) system (Amersham Corp., Arlington Heights, Ill.).

Transient Transfections and In Vitro Kinase Assays

Jurkat T cells (1.5×10⁷) were transfected as described in (Chang et al., 1998, Mol. Cell. Biol. 18:4986). Whole cell lysates derived from resting or stimulated transfectants were subjected to immunoprecipitations by the indicated antibodies, and were subjected to in vitro kinase reactions as described in (Kiefer et al., 1996, EMBO J. 15:7013 and (Sawasdikosol et al., 2003, Blood 101:3687). Anti-murine HPK1 #7 antibody was used to detect the ectopically expressed HPK wild type and mutant murine HPK1.

Results

Studies using mutant cell lines lacking TCR-associated PTKs revealed that the presence of Lck and ZAP-70 is required for HPK1 activation via TCR engagement (Liou et al., 2000 Immunity 2(4):399-408).

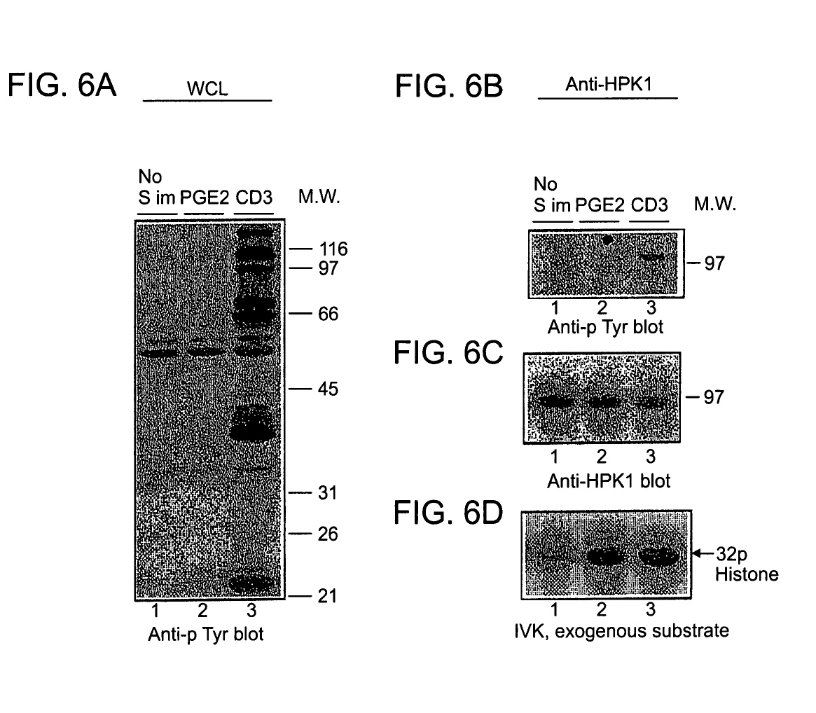

It has been reported that binding of PGE₂ to its GPCR leads to rapid activation of HPK1 kinase activity (Sawasdikosol et al., 2003, Blood 101:3687). Since other rhodopsin-like GPCRs such as the β-adrenergic receptor can utilize Lck to transduce signal via PTK-dependent pathways (Gu et al., 2000, J. Biol. Chem. 275:20726-20733), it was ascertained whether PGE₂ stimulation would engage PTKs to activate HPK1 kinase activity. First, it was assessed whether PGE₂ stimulation would induce general tyrosine phosphorylation in the Jurkat T cell line was examined. Cells were left untreated or stimulated either with 10 pM of PGE₂ or by anti-CD3ε (OKT3.14) mAb-mediated TCR crosslinking. Anti-phosphotyrosine immunoblotting of the whole cell lysates revealed no detectable change in global tyrosine phosphorylation level upon PGE₂ stimulation, whereas robust tyrosine phosphorylation was observed upon TCR crosslinking (FIG. 6A). Anti-phosphotyrosine blotting of immunoprecipitated HPK1 confirmed that, unlike a prominent tyrosine phosphorylation induced by TCR crosslinking (FIG. 6B, lane 3), stimulation by PGE₂ did not induce detectable tyrosine phosphorylation of HPK1 (FIG. 6B, lane 2). Western blot analysis using anti-human HPK1 antibody demonstrated that comparable amounts of HPK1 were present in all lanes (FIG. 6C). The immune complex in vitro kinase (IVK) assay confirmed that, both TCR and PGE₂ receptors can activate the catalytic activity of HPK1 (FIG. 6D, lanes 2 and 3).

It has been shown that Jurkat somatic mutant lines that lack Lck and ZAP-70, J.CaM1 and p116, respectively, cannot activate HPK1 upon TCR engagement (Liou et al., 2000 Immunity 12:399-408). Through the use of these mutant lines, it was assessed whether HPK1 would catalytically respond to the stimulation by PGE₂. Wild type or mutant Jurkat cell lines were left untreated or stimulated with either 10 pM PGE₂ or by antibody-mediated TCR crosslinking. These cells were lysed and the immunoprecipitated HPK1 were subjected to IVK analysis. The lost of Lck or ZAP-70 did not interfere with the ability of HPK1 to respond to PGE₂ stimulation. However, the presence of these PTKs is required for HPK1 response to TCR crosslinking. Thus, it was concluded from these studies that PGE₂ utilizes a PTK-independent pathway to activate HPK1 kinase activity.

Scaffolding proteins play a critical role in transducing activation signals from TCR to HPK1. Lat, and to a lesser extent, SLP-76 are required for TCR-induced HPK1 activation (Liou, et al., 2000 Immunity 12:399-408). To assess the role of these scaffolding proteins in PGE₂-induced HPK1 activation, mutant Jurkat T cell lines, ANJ3 and J14, which lack the expression of Lat and SLP-76, respectively, were examined for their ability to activate HPK1 in response to PGE₂ stimulation.

In these experiments, wild type Jurkat T cell line and Jurkat-derived cells containing signaling mutants were stimulated with 10 pM of PGE₂ or by TCR crosslinking. HPK1 immunoprecipitates of the samples were subjected to an in vitro kinase (IVK) assay (Sawasdikosol et al., 2003, Blood 101:3687).

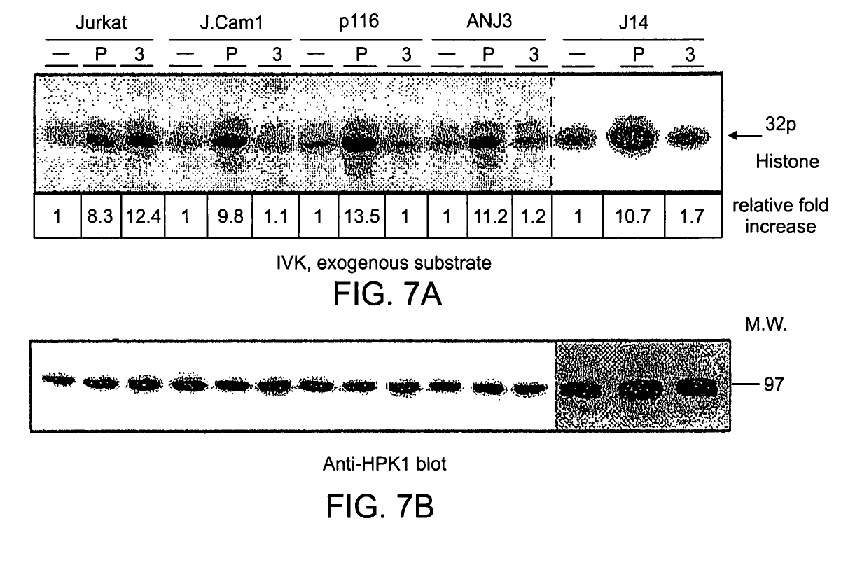

To analyze the effect of mutations in receptor-induced HPK1 kinase activity, cell lines mutant for Lck (JcaM1), ZAP-70 (p116), Lat (ANJ-3) or SLP-76 (J14) were tested for kinase activity using an IVK assay. Cells were stimulated by PGE or via TCR-crosslinking as described herein. HPK1 was immunoprecipitated from lysates and subjected to IVK assay. The relative amount of ³²P incorporated into the exogenous substrate (histone H2A) was visualized and quantitated (Storm 820 Phosphorimager, Molecular Dynamics, Eugene, Oreg.). The results are illustrated in FIG. 7A, in which the numbers under each lane represent fold increase relative to the baseline kinase activity. An anti-HPK1 immunoblot of the samples was also performed to determine amounts of HPK1 present in the samples. Analysis of the receptor-induced HPK1 kinase activity in these experiments revealed that all Jurkat cell lines could activate HPK1 kinase activity upon PGE₂ stimulation, while the mutant cell lines failed to activate HPK1 in response to TCR engagement (FIG. 7A). Western blot analysis using anti-HPK1 antibody indicated that comparable amounts of immunoprecipitated HPK1 were used in the IVK reactions (FIG. 7B).

TCR mediated signaling to HPK1 requires interaction between SH3 domain-containing adapter proteins and the proline-rich motifs of HPK1. Three of four proline-rich regions of HPK1 (P1, P2, and P4) conform to the class II consensus sequence for SH3 protein interacting domain (Liu et al., 2000, J. Immunol. 165:1417).

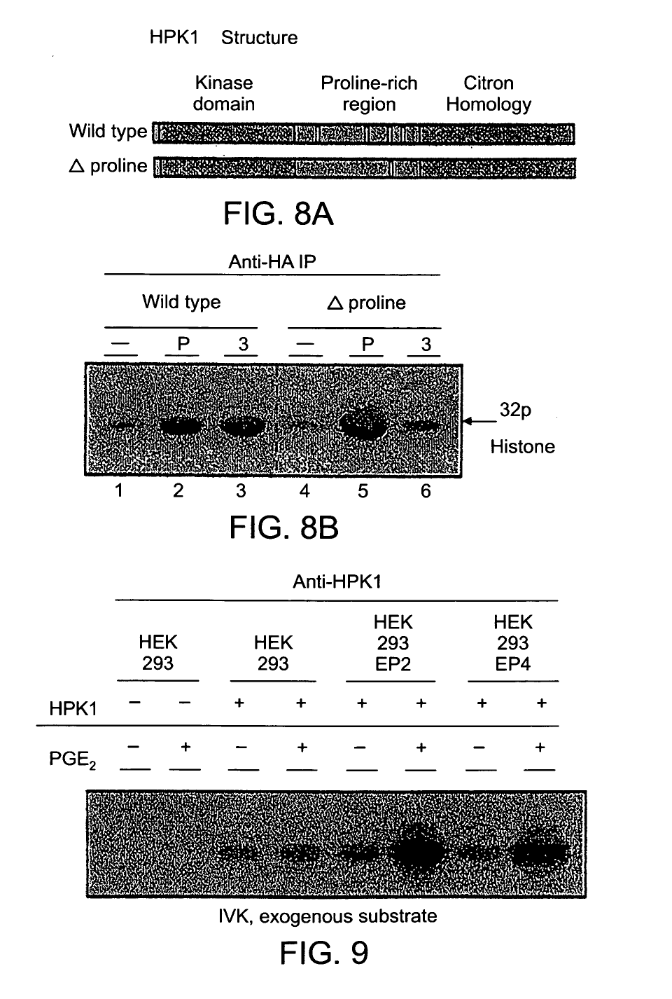

To assess the role of the proline-rich motifs in PGE₂-induced HPK1 activation, cells were transfected with constructs that encoded either the HA-tagged wild type HPK1 or a mutant form in which the P1, P2, and P4 proline-rich motifs (HA-ΔP-HPK1) were deleted (FIG. 8A). Transfectants were left untreated, stimulated by 10 pM of PGE₂, or stimulated by TCR crosslinking. The ectopically expressed HPK1 proteins were subjected to anti-HA immunoprecipitation and were subjected to IVK assay. Analysis revealed that, while the HPK1 proline-rich mutant was unable to respond to a TCR activation signal (FIG. 8B, lane 6), it responded to PGE₂ stimulation (FIG. 8B, lane 5). HA-HPK1 responded competently to both stimulations (FIG. 8B, lanes 2 and 3), suggesting that the inability of the HPK1 proline-deleted mutant was not due to epitope tagging or to overexpression of HPK1. Western blot analysis indicated that comparable amounts of HPK1 were present in all IVK reactions. This finding suggests that the proline-rich regions of HPK1, P1, P2 and P4 do not contribute to the PGE₂-induced HPK1 kinase activation. This limits the possibility that adapter proteins play a role in this process.

Prostaglandin E2 can bind with high affinity to each of the four E prostanoid receptors. The EP2 and EP4 receptors have been found in primary hematopoietic cells and hematopoietic cell lines examined.

To determine the response of the cells to PGE₂ stimulation as demonstrated by HPK1 induction. These experiments were designed to determine which of the two EP receptors can transmit an activation signal to HPK1. Briefly, a human embryonic kidney cell line that was stably transfected with either EP2 or EP4 receptor (293-EP2 and 293-EP4, respectively) (Desai et al., 2000, Mol. Pharmacol. 58:1279) was transfected with an HA-HPK1 construct. Transfectants were either left untreated or stimulated with PGE₂ as described supra. The exogenous (HA-tagged) HPK1 was immunoprecipitated using an anti-HA antibody and the immunoprecipitates were subjected to an IVK assay. HPK1 was isolated from cells and assayed

In absence of EP2 receptor and EP4 receptor, HPK1 isolated from the wild type HEK 293-EBNA cell line failed to respond to PGE₂ stimulation (FIG. 9, lane 2). HPK1 immunoprecipitates from both 293-EP2 and 293-EP4 transfectants responded robustly to PGE₂ stimulation (FIG. 9, lanes 6 and 8). HPK1 immunoprecipitated from the wild type HEK 293 cells was non-responsive to PGE₂ stimulation (FIG. 9, lane 6), as were the sham immunoprecipitates from the non-transfected control (FIG. 9, lanes 2 and 4). Comparable amounts of HPK1 were present in all lanes. These data suggest that, these receptors share the ability to activate HPK1. The use of a non-hematopoietic cells line as a model system also revealed that EP receptor signaling to HPK1 do not require any hematopoietic-cell specific factors.

Both EP2 receptor and EP4 receptor couple activation signal through the stimulatory Gα subunit (Gα_{S}) of a heterotrimeric G protein complex. The classical Gα_{S}-coupled signaling pathway relies on serine/threonine kinases (STPKs) as effectors to propagate signals. To ascertain whether HPK1 kinase activity is regulated by serine/threonine kinase-dependent pathways, HPK1 kinase activity from untreated Jurkat cells was compared to kinase activity of HPK1 from cells treated with a panel of serine/threonine and tyrosine phosphatase inhibitors.

Briefly, Jurkat cells (1×10⁷ cells) were treated with phosphatase inhibitors (CA, 100 nM calyculin A; OK, 10 nM okadaic acid; CSA, 2 μg/ml cyclosporine A; Per, 10 nM pervanadate) for 10 min. at 37° C. The cells were lysed and HPK1 was immunoprecipitated from the lysates. The HPK1 activity of the immunoprecipitates was determined by IVK assay.

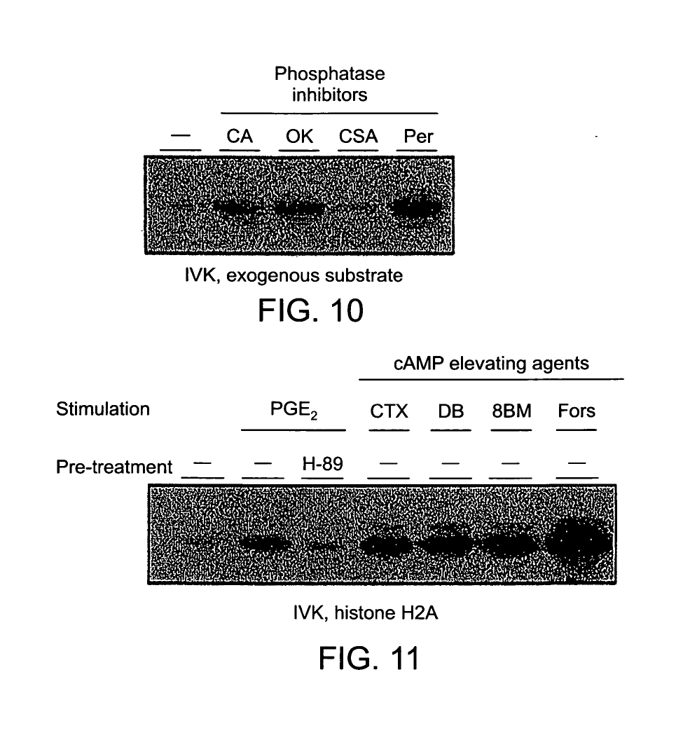

Analysis of HPK1 IVK activity revealed that both calyculin A and okadaic acid, which are both inhibitors of protein phosphatase 1 and protein phosphatase 2A, activated HPK1 kinase activity (FIG. 10, lanes 2 and 3). Cyclosporin A, a specific inhibitor of protein phosphatase 2B, failed to induce HPK1 kinase activity (FIG. 10, lane 4). Treating Jurkat cells with pervanadate, a tyrosine phosphatase inhibitor, induced a response in HPK1 catalytic activity (FIG. 10, lane 5). These data demonstrate that serine/threonine phosphatase inhibitors activate HPK1 kinase activity.

GTP-bound Gα_{S} subunit interacts with adenylyl cyclases and potentiates cAMP production. Because cAMP-dependent protein kinase A (PKA) is the dominant effector molecule downstream of Gα_{S}-coupled receptors, the question of whether PKA activity is required for the PGE₂-induced HPK1 activation was examined. To assess whether PKA activity is involved in HPK1 activation, cells were treated with pharmacological agents that activate or block PKA activity and their effect, if any, on the PGE₂ induced HPK1 activity was determined. Briefly, Jurkat cells were pretreated with H-89 (10 μM) for 1 hour prior to activation. H-89 is an isoquinolinesulphoamide drug that specifically inhibits PKA. The cells were then stimulated by PGE₂ for five min. at 37° C. Cells were lysed and an anti-HPK1 antibody was used to immunoprecipitate HPK1. Immunoprecipitated HPK1 was then assayed using the IVK to assess the role of activity. Further experiments were performed to determine the role of PKA in the PGE₂/HPK1 pathway. In these experiments, some cells were pretreated with H-89 for 30 min. before stimulating the cells with additional reagents. Stimulation of cells with the additional reagents (in H-89 pretreated cells and in cells that were not pretreated) was for 10 min. at 37° C. Lysates were then prepared from these cells and HPK1 was recovered from the cells using immunoprecipitation and assay using the IVK reaction. Briefly, Jurkat cells (1×10⁷ cells) were left untreated or were pre-treated for one hour at 37° C. with 10 μM of H-89 (PKA inhibitor) and subsequently stimulated with CTX (1 μg/ml), DB (100 μM adenosine-3′,5′-cyclic monophosphate, N⁶, O²,-dibutyryl-, sodium salt), 8BM (100 μM adenosine-3′,5′-cyclic monophosphate, 8-bromo-, sodium salt); forskolin (50 μM). The cells were lysed and HPK1 was then immunoprecipitated from the lysates and the HPK1 activities of the cells were determined by IVK assay. Anti-HPK1 immunoblots were also generated to determine the amounts of HPK1 in each sample.

Pretreatment of cells with H-89 blocked PGE₂-induced HPK1 activity (FIG. 11, lane 3). Consistent with this observation, treating Jurkat cells with known activators of PKA such as cholera toxin, cell-permeable cAMP analogues, and forskolin, robustly induced HPK1 kinase activity (FIG. 11A, lanes 4-7). Western blot analysis revealed that comparable amounts of HPK1 were present in all lanes. These data demonstrate that activation of PKA activates HPK1 kinase activity, and that PKA activity is necessary for activating HPK1 activity. Accordingly, certain compounds that modulate HPK1 activity are compounds that modulate PKA activity, and vice versa.

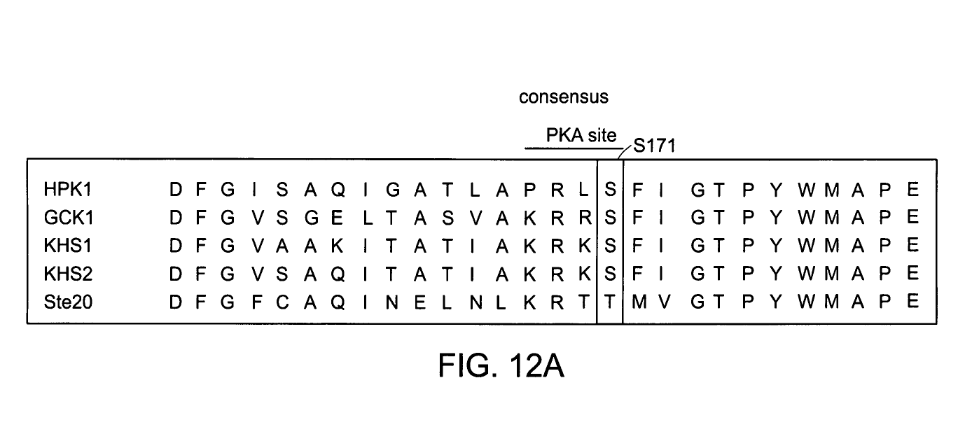

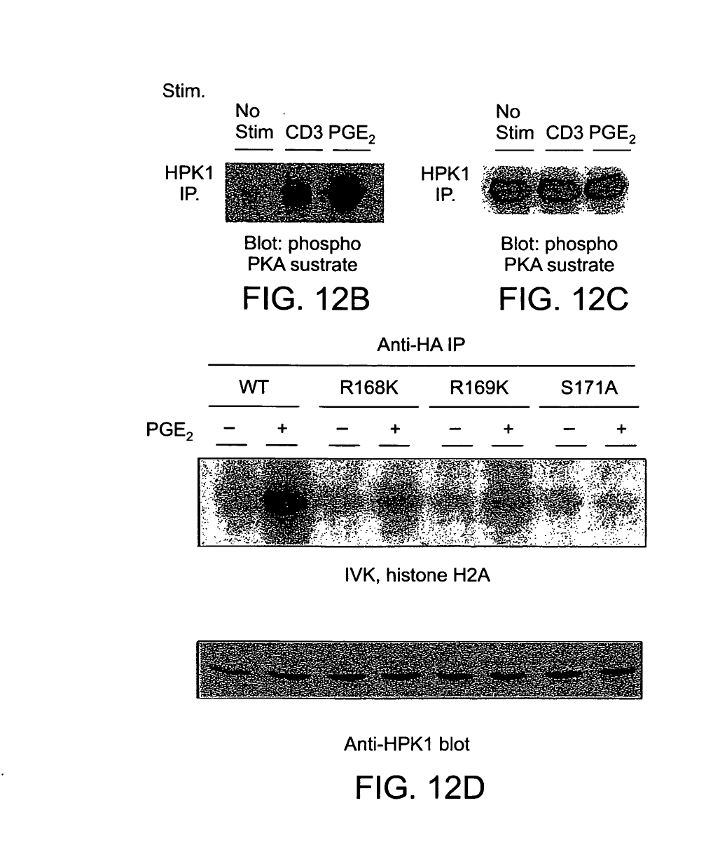

The susceptibility of PGE₂-induced HPK1 kinase activity to a PKA inhibitor, in conjunction with the catalytic responsiveness of HPK1 to cAMP elevating agents, indicates that PKA is an upstream regulator of PGE₂-induced HPK1 activation. The primary sequence of HPK1 was examined for the preferred PKA phosphorylation site on HPK1. Within the activation loop of HPK1, there exists a perfect PKA phosphorylation site at serine 171 (FIG. 12A). This serine is conserved in all members of the KHS subfamily of STE20 kinases. To determine whether PKA can directly phosphorylate HPK1 upon stimulation by PGE₂, experiments were conducted using an anti-phospho PKA substrate antibody that recognizes the arginine-based motif that is present at sites that are phosphorylated by PKA (FIG. 12A).

In these experiments, Jurkat T cells were stimulated with PGE₂ for 10 min., lysed, and immunoprecipitated HPK1 was subjected to Western blot analysis using anti-phosphorylated PKA substrate antibody. Western blot analysis revealed that HPK1 (HPK1 IP.) from non-stimulated cells was not recognized by an antibody that recognizes phosphorylated PKA substrate (FIG. 12B, lane 1), but the same antibody recognized the phosphorylated HPK1 upon stimulation by PGE₂ (FIG. 12B, lane 3). Similarly, the antibody also recognized HPK1 immunoprecipitated from cells that were treated with anti-CD3 (FIG. 12B, lane 2). When the Western blot membrane was stripped and reprobed with the anti-HPK1 antibody, a comparable amount of HPK1 was present in all lanes (FIG. 12C).

These data demonstrate that phosphorylation of serine 171 by PKA is a necessary event for activation of HPK1.

The recognition of PGE₂-activated HPK1 by the anti-phosphorylated PKA substrate antibody led to an analysis of HPK1 primary sequence for the presence of the optimal consensus PKA motif: (the amino acid sequence RRXS/T, where X represents any amino acid (Pearson et al. 1991, Methods Enzymol 200:62). Sequence analysis identified serine 171, located within the activation loop of the kinase domain, as the only optimal PKA site in HPK1 (FIG. 12D). Further analysis revealed that, while the arginine residue at the −2 position relative to the serine 171 (arginine 169) is conserved in all KHS family members, only HPK1 possess an arginine at the −3 (arginine 168) position relative to serine 171. The conserved double arginine sequence was also found in murine HPK1 sequence, but not in the majority of Ste20 orthologs.

To assess the importance of arginine 168, arginine 169 and serine 171 in PGE₂-induced HPK1 activation, a panel of mutant HPK1 expression constructs that encode a point mutation at each of these amino acids was created. In these mutants, arginines were changed to lysines and serine was changed to alanine. These mutant HPK1 constructs were transfected into Jurkat cells and the ectopically expressed HA-tagged HPK1 were immunoprecipitated from resting or PGE₂ stimulated cells.

Analysis of HPK1 kinase activity revealed that mutation of serine 171 ablated the response of HPK1 to PGE₂ stimulation signals. Arginine to lysine mutations at either residue 168 or 169 of HPK1 reduced the responsiveness to PGE₂ stimulation by approximately 90 percent. This response to PGE₂ was different from the complete loss of basal kinase activity observed when threonine 175, a residue conserved throughout all Ste20 family members, was mutated to alanine. These data suggest that the kinase that phosphorylates serine 171 requires both arginines 168 and 169 to direct its substrate specificity. Therefore, compounds that block access to one or more of serine 171, arginine 168, or arginine 169 are useful for decreasing activity of HPK1.

In summary, these experiments show that PGE₂ utilizes the E prostanoid 2 (EP2) receptor and E prostanoid 4 (EP4) receptor to generate a cyclic AMP-dependent pathway that in turn activates HPK1. Blocking cAMP-dependent protein kinase A using the inhibitor H89 that blocks PKA activity blocks PGE₂-induced HPK1 activation. Consistent with this finding, treatment of Jurkat cells with cAMP elevating agents activates HPK1 kinase activity. In addition, a mutation of serine 171, the locus that forms the PKA phosphorylation site within the activation loop of HPK1 to alanine, prevents the mutant from responding to PGE₂-generated stimulation signal. Thus, HPK1 can be activated by a cAMP-dependent pathway, and PKA-mediated phosphorylation of serine 171 in the activation loop of HPK1 appears to be the mechanism controlling this process.

### Example 3

### HPK1, a Downstream Effector of Cbl-b, Negatively Regulates T cell Activation

Animals

Cbl-b^{-/-} mice were obtained from Dr. H. Gu (Columbia University, New York, N.Y.) (Chiang et al., 2000, Nature 403:216). HPK1^{-/-} mice were also used in these experiments. Both types of mutant mice were backcrossed to a C57BL/6 background for at least 9 generations. Wild type C57BL/6 mice were purchased from Jackson Laboratories (Bar Harbor, Me.)

Molecular Constructs

The pCEFL vector encoding wild type or the C373A ligase-defective mutant Cbl-b constructs were obtained from Dr. S. Lipkowitz of the National Cancer Institute (Bethesda, Md.) (Ettenberg et al., 2001, J. Biol. Chem. 276:27677). The pcDNA3 vector encoding the HA-tagged, murine HPK1 was obtained from Dr. F. Kiefer and is described in (Kiefer et al., 1996, EMBO J. 15:7013). A FLAG-tagged human HPK1 was obtained from Dr. T-H Tan, (Baylor College of Medicine, Houston, Tex.) and is described in (Hu et al., 1996, Genes Dev. 10:2251. A kinase domain-deleted mutant of murine HPK1 molecule that is missing the first 291 amino acids from the N-terminus was created by PCR-assisted amplification and sub-cloned into the Nde1/Cla1 sites of the pEBB-HA vector. Wild type, SH2 (R38V) and SH3 (W169L) rat Crk II cDNAs were obtained from Dr. M. Matsuda (Research Institute for Microbial Diseases; Osaka, Japan) and is described in Tanaka et al. (1994, Proc. Natl. Acad. Sci. USA 91:3443). These sequences were sub-cloned into the KpnI/BamHI sites of the pEBB-HA vector. Luciferase reporter constructs regulated by either the IL-2P or the 3×NFAT/AP-1 enhancer element (nucleotide position relative to the IL-2 gene transcription start site: −288 to −268) were as described in Chang et al., 1998, Mol. Cell. Biol. 18:4986-4993. pSUPER-based constructs encoding small hairpin precursors for RNAi that target HPK1 transcripts were created by ligating the following primers and their complementary strands into the Bgl II/Xho I— sites of the linearized vector: HPKl-RNAi-1: 5′gatccccTAGAGACCCCCGGGACCACttcaagagaGTGGTC CCGGGGGTCTCTAtttttggaaa3′ (SEQ ID NO:5);

HPK1-RNAi-2: 5′gatccccTTCTGTGGGGCTGGTTCTCTtcaagagaGAGAACCAGCCCCACAGAAtttttggaaa3′ (SEQ ID NO:6). Upper case letters denote the predicted upper and lower strands, respectively, of the processed RNAi duplex.

Antibodies and Other Reagents

The following anti-HPK1 antibodies were used in these studies: rabbit pAb #47 (Sawasdikosol et al., 2003, Blood 101:3687) was used to immunoprecipitate and Western blot human HPK1; rabbit pAb #5 and 6 (Kiefer et al., 1996, EMBO J. 15:7013) were used to immunoprecipitate murine HPK1, and pAb #7 (Kiefer et al., 1996, EMBO J. 15:7013) was used to detect murine HPK1 by Western blot (Kiefer et al., 1996). A 12CA5 anti-hemagglutinin mAb and the anti-human CD3ε (OKT3.14) mAb were generated in the laboratory using methods known in the art (see, e.g., Current Protocols in Immunology, Vol. 2, Coligan et al., National Institutes of Health, 1991). Anti-murine CD3 (OKT3.14 clone was used, which recognizes only the human CD3 molecule and does not cross react with the mouse CD3 molecule) and anti-murine CD28 antibodies were purchased from (BD/Pharmingen; San Jose, Calif.). Anti-hamster Ig was purchased from Southern Biotechnology (Birmingham, Ala.) γ-[³²P]-ATP was obtained from Perkin Elmer Life Sciences/NEN (Boston, Mass.). Histone H2A, the exogenous substrate used in in vitro kinase reactions, was purchased from Roche Applied Science (Indianapolis, Ind.).

Transient Transfection and Luciferase Reporter Assay

Constructs encoding a luciferase reporter gene whose transcription is regulated by either the −140 NFAT/AP-1 enhancer element of the human IL-2P (Chang et al., 1998, Mol. Cell. Biol. 18:4986-4993) or the entire human IL-2P (the promoter that controls the IL-2 gene transcription) were used as reporters of gene transcription. Jurkat cells (1.5×10⁷) were transfected as described in Chang et al. (1998, supra), using 2.5 μg of either of the two reporters described above, along with 100 ng of pNull Renilla luciferase reporter construct and 10 μg of the indicated experimental constructs. Transfectants were either not stimulated or stimulated with 12-myristate 13-acetate and CD3. In these experiments, 1 μg of the anti-human CD3 (OKT3 antibody) was prepared in the solution of phosphate buffered saline (PBS) (1 μg/ml) and used to coat a 48 well tissue culture plate. Stock PMA was added along with the cell suspension during the stimulation, resulting in a final concentration of 10 ng/ml. Incubation was for six hours. Dual luciferase assays were performed according to the manufacturer's instructions (Promega, Madison, Wis.) and the relative luciferase activity was measured using a Sirius luminometer (Berthold Detection System, Pforzheim, Germany). In experiments assaying the activity of the transfected HPK1, antibody recognizing the hemagglutinin (HA) tag was used to immunoprecipitate HA-tagged HPK1, which was subjected to an in vitro kinase reaction as, described in Kiefer et al., (1996, EMBO J. 15:7013-7025).

Cells and Stimulation Conditions

The J77 Jurkat T cell line was grown in RPMI 1640 complete medium (RPMI 1640 supplemented with 10% fetal bovine serum, 2 mM L-glutamine and 100 units of penicillin/streptomycin). For stimulation by anti-CD3-antibody-mediated TCR crosslinking, Jurkat cells were suspended in tissue culture plates coated with 1 μg/ml anti-human CD3ε (OKT3.14). For stimulation of murine splenocytes, cells were stimulated with 1 μg/ml plate-bound anti-CD3 Ab (2C11-145, 10), in the presence of 1 μg/ml soluble anti-CD28 Ab (BD/Pharmingen) or 1 μg/ml ConA (Sigma, St. Louis, Mo.). For cell expansion, equal numbers of cells were stimulated as described above in the presence of 20 iU/ml IL-2 (NIH, Rockville Md.) for one week. To stimulate IL-2 production, primed cells were re-stimulated as described above for 20 hours, and supernatants were recovered for determination of IL-2 by ELISA. Other cell cultures received GolgiStop™ (BD/Pharmingen) for the last six hours of stimulation to block cytokine secretion prior to intracellular staining.

Proliferation Assays

Cells were cultured in 96 well plates under different stimulatory conditions for three days and were pulsed with 1 μCi of [³H] thymidine/well for the last 18 hours of stimulation. Cells were then harvested on a glass-fiber filter (Wallac, Turuk, Finland) and incorporated [³H] thymidine was determined using a liquid scintillation counter (Wallac). The results were expressed as the mean of triplicate cpm±SD.

IL-2 ELISA and Intracellular IL-2 Staining

A pair of monoclonal antibodies recognizing different IL-2 epitopes (BD/Biosciences, San Diego, Calif.) were used to quantitate the level of IL-2 present in supernatants using a standard ELISA method. For intracellular IL-2 staining, cells were stained for cell surface markers (anti-CD4 FITC, antibody and anti-CD8 CyChrome antibody, Pharmingen, Franklin Lakes, N.J.) and then permeabilized and stained with an anti-IL-2 PE antibody. Cells were analyzed in a BD FACScalibur™, using the CellQuest™ program.

Results

Whether ubiquitin ligase-independent mechanisms are involved in Cbl-b-mediated inhibition of TCR-induced IL-2 gene transcription was investigated using Jurkat T cells as a model system. In addition to the regulation of the entire IL-2P the effect of Cbl-b on the NFAT/AP-1 enhancer element regulating a luciferase reporter gene was examined. A panel of constructs encoding Cbl-b, a ubiquitin ligase-defective mutant (C373A) or the C373A mutant with tyrosine to phenylalanine mutations at the major TCR-induced phosphorylation sites, residues 665 and 709 (C373A/2Y>F), were transfected along with either the IL-2P-regulated or the NFAT/AP-1-regulated luciferase reporter gene into Jurkat T cells. Transfectants were unstimulated or stimulated by plate-bound anti-CD3 mAb in the presence of the phorbol ester PMA (CD3+PMA). T cell stimulations were done using CD3+PMA because IL-2 gene transcription responds robustly to this form of stimulation. Furthermore, because PMA is known to bypass Cbl-mediated inhibition of AP-1 activity (Rellahan et al., 1997, J. Biol. Chem. 272:30806-30811), stimulation with PMA ensured that any inhibitory effect observed on NFAT/AP-1 activity would be attributable to the effect on NFAT transcriptional activity.

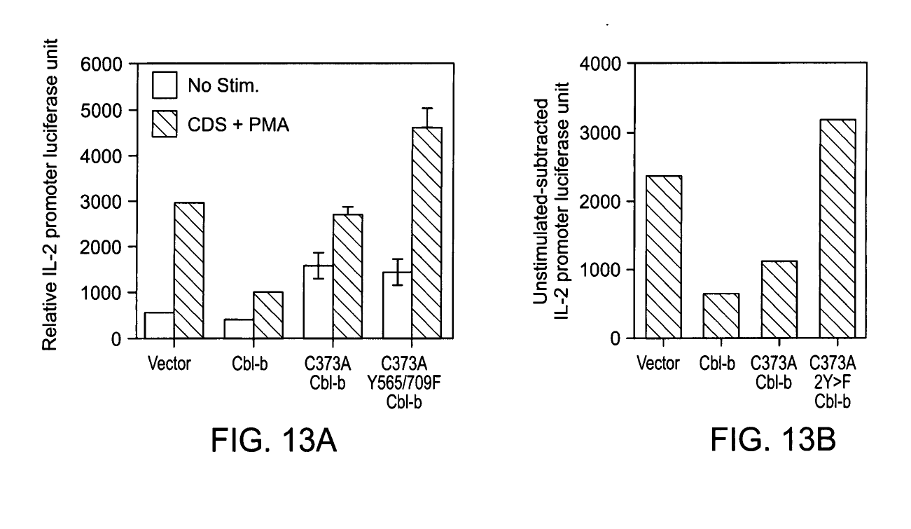

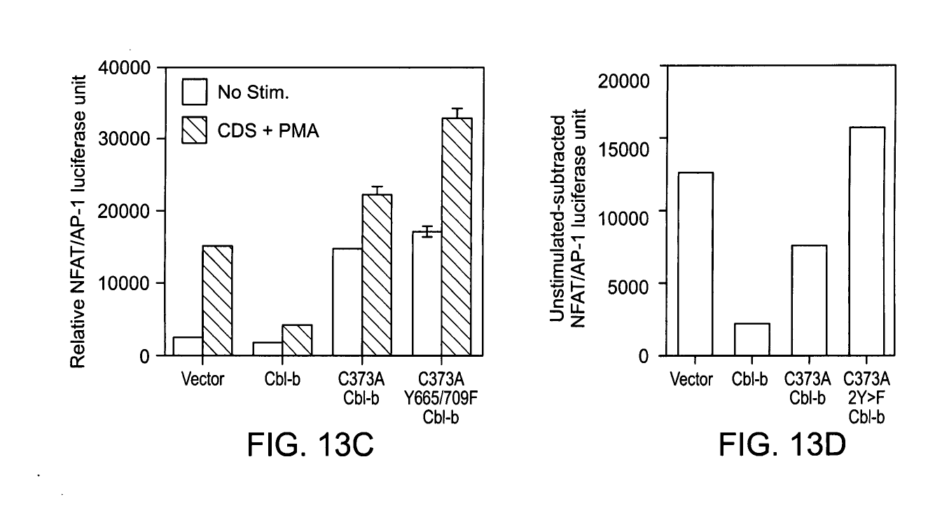

Comparison of the luciferase activities among transfectants revealed that the expression of the ligase-defective Cbl-b mutants caused cells to transcribe the IL-2 gene at a higher basal rate, even in the absence of any stimulation (FIG. 13A). These mutants still had their tyrosine kinase binding (TKB) domains intact. Therefore, they may interfere with the ability of endogenous Cbl to bind its target proteins. A similarly elevated basal NFAT activity was observed previously in unstimulated transfectants expressing the ligase defective oncogenic form of c-Cbl, 70Z (Liu et al., 1997, J. Biol. Chem. 272:168-173; Zhang et al., 1999, J. Biol. Chem. 274:4883-4889). Furthermore, it was observed that unstimulated T cells from c-Cbl and Cbl-b double knockout animals spontaneously proliferate (Naramura et al., 2002, Nat. Immunol. 3:1192-1199). These results suggest that Cbl-b plays a critical role in maintaining cellular homeostasis.

Upon stimulation by CD3⁺ PMA, transfectants expressing wild type Cbl-b were markedly inhibited in TCR-induced IL-2P activity compared to controls that were transfected with empty vector (FIG. 13A). While the CD3⁺ PMA-induced luciferase activity in transfectants expressing the C373A ligase defective Cbl-b regained some of their ability to produce IL-2 in response to TCR engagement, the level of TCR-induced IL-2P activity increased further when tyrosine to phenylalanine mutations at residues 665 and 709 were introduced into the C373A mutant (FIG. 13A). These tyrosine residues are the primary tyrosine phosphorylation sites in Cbl-b and facilitate its TCR-induced interaction with the Crk family of SH2/SH3-domain containing adapter proteins.

By subtracting the elevated activity observed in unstimulated transfectants from TCR-induced IL-2P activity, it becomes clear that the ligase-defective Cbl-b mutant maintains significant ability to inhibit TCR-induced IL-2P activity and that the Crk binding sites at tyrosines 665 and 709 make an important contribution to the inhibition of TCR activation (FIG. 13B). A similar pattern was also observed when identical experiments were performed using the NFAT/AP-1 reporter (FIG. 13C and FIG. 13D), suggesting that suppression of TCR-induced NFAT activity is a component of the ubiquitin ligase-independent, Cbl-b-mediated negative regulation of IL-2P activity.

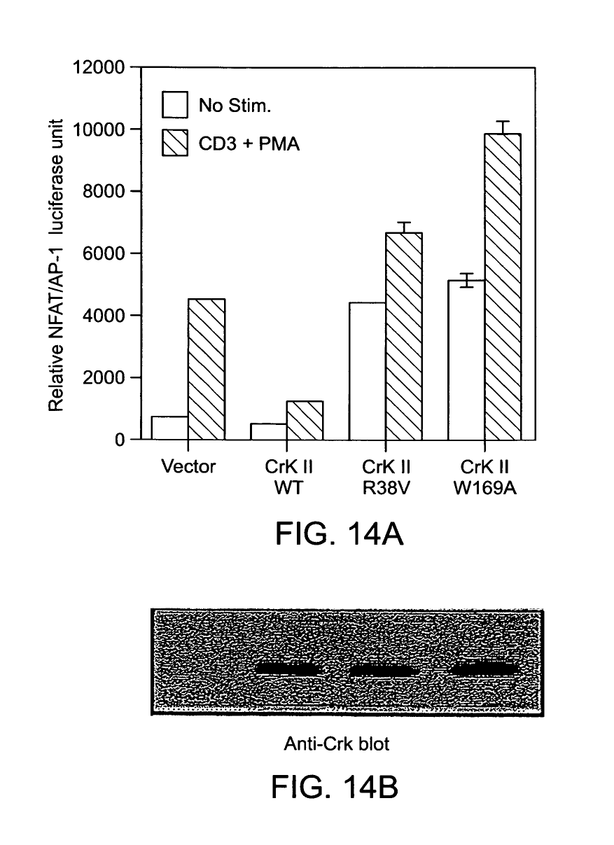

The Crk family of SH2 and SH3 domain-containing adapter proteins binds to tyrosine phosphorylated c-Cbl upon antibody-mediated TCR crosslinking (Buday et al., 1996, J. Biol. Chem. 16:6159-6163; Reedquist et al., 1996, J. Biol. Chem. 271:8435-8442; Sawasdikosol et al., 1996, J. Immunol. 157:110-116). Since identical TCR-induced associations also occur between Cbl-b and Crk, it was determined whether Crk plays a role in ubiquitin ligase-independent, Cbl-b-mediated negative regulation of TCR-induced signaling. Wild type Crk, or its mutants with defects in either their SH2 (R38V) or SH3 (W 169L) domain, were ectopically expressed and their effect on TCR-induced NFAT/AP-1 activity was determined. It was observed that ectopic expression of wild type Crk inhibited NFAT/AP-1-regulated luciferase activity, whereas overexpression of Crk mutants with defects in either the SH2 or SH3 domain augmented TCR-induced NFAT/AP-1 activity compared to a vector control (FIG. 14A). Western blot analysis revealed that comparable amounts of Crk protein were expressed in all transfectants (FIG. 14B). These data suggest that the selective loss of protein interactions by these Crk mutants enable them to function as “dominant interfering” molecules that can compete with endogenous Crk and perhaps with other members of the Crk family, interfering with Crk-dependent negative regulation of the NFAT/AP-1 enhancer element. Thus, overexpression of these mutants results in responses that are greater than seen with a vector control, suggesting they are inhibiting the binding of endogenous Crk.

Crk is one of the adapter proteins that couples the TCR-generated signal to HPK1. Increasing the amount of wild type Crk in the cell therefore increases the strength of the activation signal to HPK1. A Crk mutant that does not bind the upstream activator (Cbl-b) of HPK1 or a mutant that fails to bind HPK1 can act as dominant interfering molecule that sequesters HPK1 or Cbl-b from interaction with endogenous Crk protein. This results in a decrease in HPK1 kinase activity. Since HPK1 is a negative regulator of NFAT activity, the decrease in HPK1 kinase activity results in an increase in TCR-induced NFAT activation.

Adapter proteins are devoid of intrinsic catalytic activity and rely on their interacting partner(s) to carry out effector functions. The SH2 domain of Crk associates with tyrosine-phosphorylated Cbl-b upon TCR engagement (Elly et al., 1999, Oncogene 18:1147). Experiments were conducted to determine if there were Crk SH3-binding proteins other than C3G, the guanine nucleotide exchange factor for RaplA, capable of carrying out the inhibitory function of Cbl-b. RaplA is thought to inhibit Raf leading to inhibition of ERK MAPK (Kitayama et al., 1989, Cell 56:77) activity, but the Crk/C3G/RaplA pathway should not affect NFAT activity. Therefore, to identify other proteins that bind to the SH3 domain of Crk in T cells, Jurkat cells were labeled with ³⁵S-methionine and GST-Crk SH3 fusion proteins were used to pull down Crk SH3-associated proteins from labeled lysates. Crk-associated proteins were resolved by SDS-PAGE and then visualized by autoradiographic imaging.

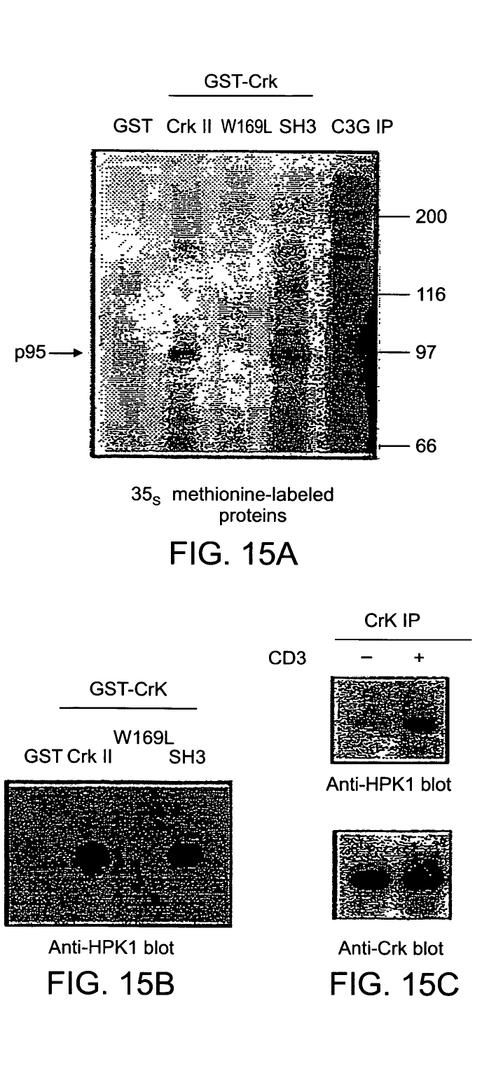

Analysis revealed that GST-Crk SH3 pulled down a prominent band that corresponded to a protein with a relative mobility of 95 kDa (FIG. 15A, lane 1), termed “p95”. GST-Crk with a non-functional SH3 domain (W 169L) did not bind this protein (FIG. 15A, lane 2), demonstrating that p95 was an SH3-associated protein (FIG. 15A, lane 3). The intensity of the p95 autoradiographic band, relative to the intensity of the well-characterized Crk-associated protein C3G (FIG. 15A, lane 4), suggested that more p95 bound to the Crk SH3 domain than did C3G. To identify the p95 protein, whole cell lysates prepared from approximately 1×10⁹ Jurkat cells were incubated with glutathione-bound GST-Crk SH3 domain. Proteins associated with the Crk SH3 domain were resolved by SDS-PAGE and visualized by silver staining. The 95 kDa band was excised and its identity was determined by MALDI-TOF. Analysis identified the 95 kDa protein as HPK1—a hematopoietic cell-restricted member of the Ste20 serine/threonine kinase family. Western blot analysis using an anti-HPK1 antibody confirmed that this protein co-precipitated with a GST-Crk SH3 pull down (FIG. 15B).

To further examine the intracellular interactions of HPK1, about 1×10⁹ Jurkat cells were stimulated by 1 μg of soluble anti-CD3-mediated receptor and crosslinked at 37° C. for 10 min. Cells were lysed and Crk adapter proteins were immunoprecipitated and analyzed for co-precipitated proteins. FIG. 15C demonstrates that HPK1 was present in the immunoprecipitate (using an anti-HPK1, upper panel) and therefore was associated with Crk (identified using an anti-Crk, lower panel). These data demonstrate a constitutive interaction between endogenous HPK1 and Crk in Jurkat cells. The interaction was enhanced upon TCR engagement (FIG. 15C). These data indicate that HPK1 plays a role in the regulation of T cell activity by Cbl via the Crk adapter protein.

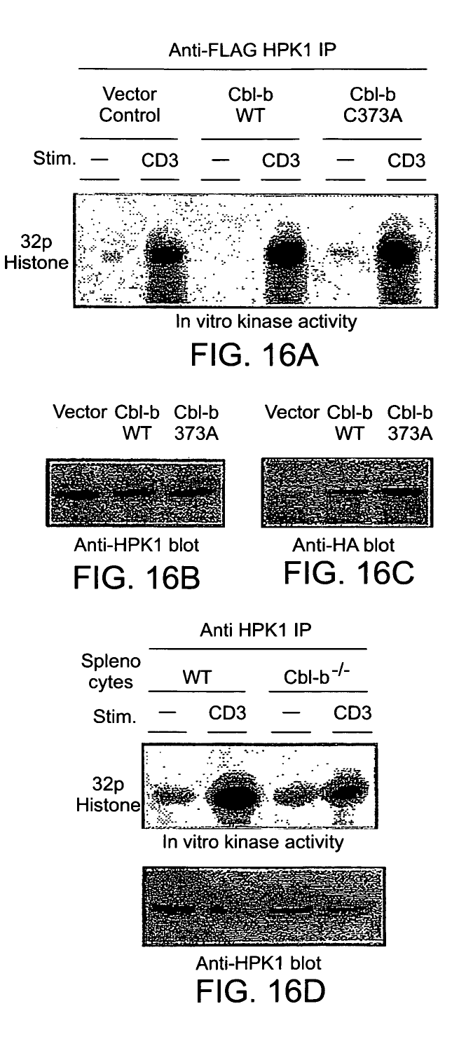

To further assess the relationship of Cbl-b and HPK1 activity, HPK1 was co-transfected with either the wild type or the ubiquitin ligase-defective Cbl-b construct. Whether TCR-induced HPK1 kinase activity was altered by Cbl-b overexpression was then examined. Transfected HPK1 was immunoprecipitated by its FLAG-epitope tag from whole cell lysates of unstimulated or TCR-stimulated transfectants. Immunoprecipitated HPK1 was subjected to an in vitro kinase assay to determine its kinase activity. Analysis revealed that overexpression of either form of Cbl-b rendered HPK1 catalytically more responsive to TCR signals (FIG. 16A), suggesting that HPK1 activation occurred by a mechanism independent of ligase activity. HPK1 Western blot analysis of immunoprecipitated HPK1 revealed that comparable amounts of HPK1 were present in all kinase reactions (FIG. 166B). Cbl-b Western blots of whole cell lysates from transfectants also revealed that comparable amounts of Cbl-b were expressed (FIG. 16C).

The experiments presented above in Jurkat cells show that Cbl-b regulates the TCR-induced activation of HPK1. To further assess this relationship, splenocytes were isolated from wild type or Cbl-b^{-/-} mice and the effect of the loss of endogenous Cbl-b on TCR-induced HPK1 kinase activity was assessed. Splenocytes were either untreated or were stimulated by anti-CD3-mediated antibody crosslinking. HPK1 was immunoprecipitated from splenocytes and HPK1 in vitro kinase activity was assessed. Analysis of HPK1 kinase activity from wild type and Cbl-b^{-/-} splenocytes revealed that HPK1 from Cbl-b^{-/-} splenocytes achieved only 28% of the activity of HPK1 from wild type cells when stimulated by TCR crosslinking (FIG. 16D, upper panel). Western blot analysis using an anti-murine HPK1 antibody revealed that comparable amounts of immunoprecipitated HPK1 were present in all lanes (FIG. 16D, lower panel). These data suggest that Cbl-b relays TCR-generated signals resulting in the activation of HPK1. Accordingly, compounds that modulate Cbl-b expression or activity are useful for modulating HPK1 activity.

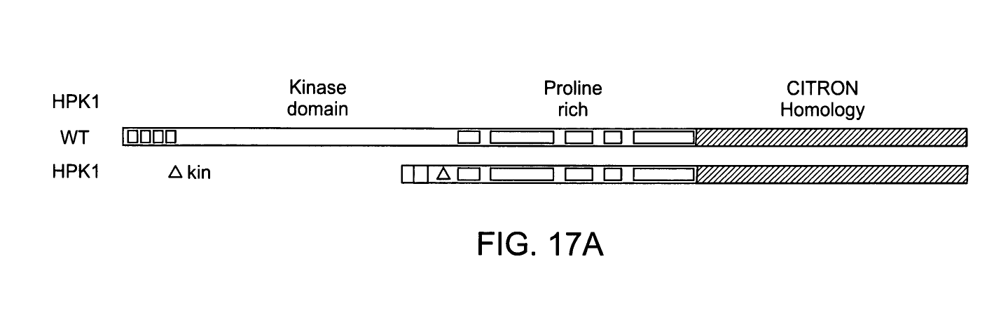

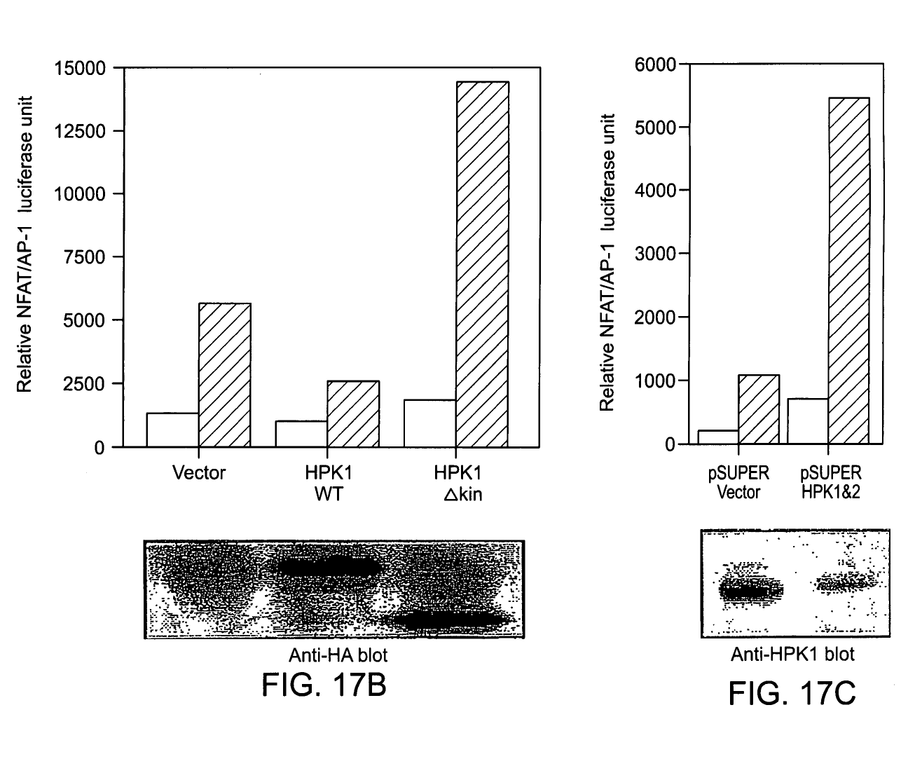

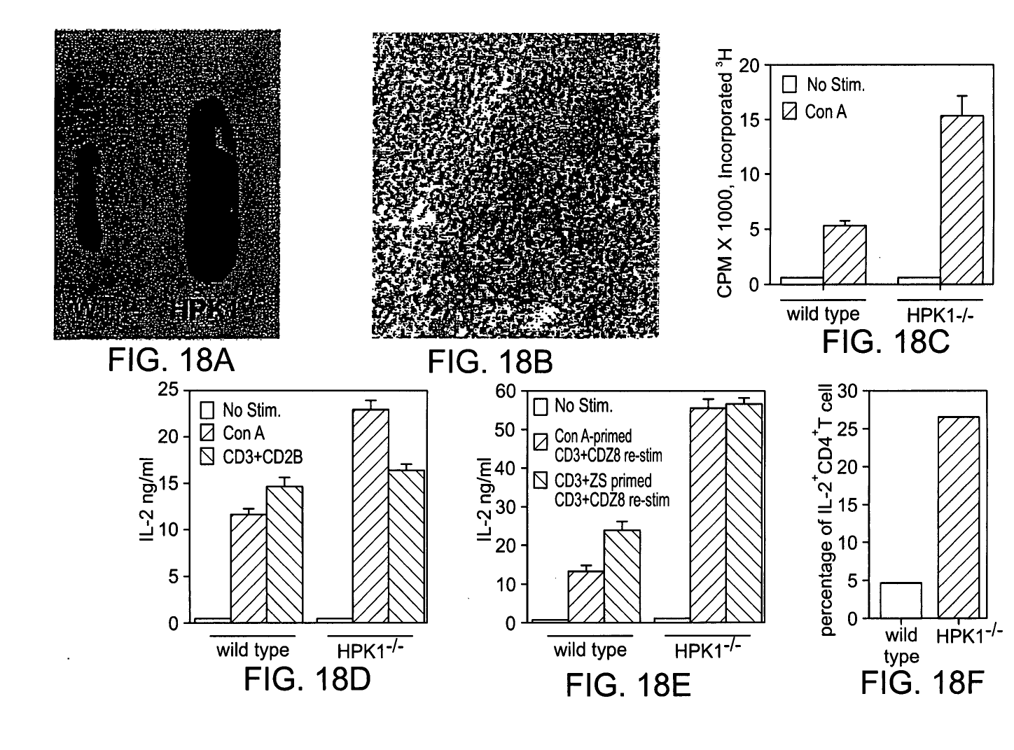

The effect of HPK1 overexpression on the NFAT/AP-1 response to TCR stimulation was assessed using a luciferase reporter construct. In these experiments, wild type HPK1 or its kinase domain-deleted mutant (HPK1 Δkin) (FIG. 17A) was transfected into Jurkat T cells and their effect on TCR-induced NFAT/AP-1 activation was determined by assessing luciferase expression. Analysis of the luciferase reporter activity revealed that overexpression of wild type HPK1 suppressed NFAT/AP-1 activity, whereas overexpression of the mutant HPK1 Δkin augmented the NFAT/AP-1 response to TCR activation signals (FIG. 17B). The mutant HPK1 appeared to interfere with endogenous HPK1 in a dominant negative fashion, since Jurkat cells expressing this mutant exhibited a stronger response to anti-CD3⁺PMA stimulation when compared to control transfectants that received the vector alone. Thus, these data suggested that the mutant was interfering with the function of endogenous HPK1.

To complement the overexpression study, an RNAi-mediated translational suppression system was used to assess the impact of the loss of endogenous HPK1 on TCR-induced NFAT/AP-1 activation. Using the pSUPER RNAi System™, a plasmid-based system, to express small hairpin RNAs (shRNAs) with complementarities to the human HPK1 mRNA transcript, the ability of the HPK1 RNAi transfectants to drive the NFAT/AP-1-regulated luciferase reporter in response to stimulation by anti-CD3⁺ PMA was assessed. Transfectants expressing HPK1 siRNA responded to TCR stimulation five fold better than transfectants that received a vector control (FIG. 17B). Whole cell lysates of these transfectants were resolved by SDS-PAGE and immunoblotted with the anti-HPK1 antibody to determine the expression levels of endogenous HPK1. Analysis revealed that cells that expressed HPK1 RNAi targeting constructs lost much of their HPK1 expression when compared to the control transfectants (FIG. 17B). Thus, the reduction of HPK1 expression correlated with enhanced TCR-induced NFAT/AP-1 activity, consistent with HPK1 functioning as a negative regulator of TCR-induced NFAT activation. HPK1^{-/-} mice were generated as described supra for these studies.

Thymocytes from HPK1^{-/-} animals were analyzed for possible defects in T cell development, and no skewing was found in cell surface markers (CD4, CD8, CD25, CD69, and CD45) when compared to wild type thymocytes. While no gross defects were found in thymic T cells, splenomegaly was noted in HPK1^{-/-} animals that were older than six months of age (FIG. 18A).

To further characterize the nature of splenic enlargement, spleens were sectioned for histologic analysis and stained with hematoxylin and eosin (H&E). Histological analysis revealed that mononuclear cell infiltrates comprised of lymphocytes, megakaryocytes, neutrophils, and plasma cells were found in the HPK1^{-/-} spleens. The “wall to wall” infiltration of these cells disrupted the normal red and white pulp architecture found in normal spleens (FIG. 18B). The presence of multiple cell types in the infiltrated spleen suggests that HPK1 may play an important role in controlling the proliferation of other hematopoietic cell types.

The impact of the loss of HPK1 on peripheral T cell function was also investigated. First, the ability of wild type and HPK1^{-/-} splenocytes to proliferate in response to stimulation by a T cell mitogen, concanavalin A (ConA) was determined. After 48 hours of stimulation by ConA, HPK1^{-/-} splenocytes had proliferated two-fold more than their wild type counterpart (FIG. 18C). A comparison was also made between the level of IL-2 produced by wild type and HPK1^{-/-} splenocytes using the same ConA stimulating conditions. It was found that HPK1^{-/-} splenocytes produced twice as much IL-2 as their wild type counterpart (FIG. 18D). In view of these findings, no significant differences in IL-2 production were found when HPK1^{-/-} splenic T cells were stimulated by antibody-mediated crosslinking of the CD3 and CD28 receptors (CD3⁺ CD28) (FIG. 18D). However, if primary T cells were first stimulated with either ConA or by CD3⁺ CD28 engagement for 5 days, the activated T cells from HPK1^{-/-} animals responded to anti-CD3⁺ anti-CD28 re-stimulation by producing four-fold more IL-2 than wild type T cells (FIG. 18E).

To determine whether the enhanced IL-2 production was due to greater production of IL-2 per cell or was a reflection of more cells producing IL-2, anti-CD3⁺ anti-CD28-primed wild type and HPK1^{-/-} CD4⁺ T cells were stained to detect intracellular levels of IL-2. FACS analysis revealed that there is an increase in the number of CD4⁺ HPK1^{-/-} T cells that produce IL-2 in response to anti-CD3⁺ anti-CD28 restimulation, compared to wild type T cells (FIG. 18F). Further analysis revealed that HPK1^{-/-} T cells also possessed a slightly higher mean fluorescent intensity than that exhibited by the CD4⁺ wild type T cells, suggesting that on a per cell basis HPK1^{-/-} T cells may also produce more IL-2.

The data in these experiments demonstrate that HPK1 is a downstream effector of Cbl-b and the activation of HPK1 results in the negative regulation of TCR-induced NFAT activation and IL-2 transcription. These results are consistent with the observation that HPK1^{-/-} T cells respond more robustly to stimulation by ConA or to CD3⁺CD28 antibody engagement.

In view of these data, compounds that decrease HPK1 expression or activity are useful for increasing TCR-induced NFAT activation and increasing IL-2 production. In addition, such compounds are useful for increasing T cell responses.

### Example 4

### HPK1 Modulation of PGE₂-Immune Suppression in Lymphocytes

As demonstrated herein, PGE₂ can induce HPK1 kinase activity in Jurkat T cells. To further investigate the role of HPK1, the effects of PGE₂ in primary lymphocytes were examined in the presence and absence of HPK1.

Antibodies, Media, and Reagents

Rabbit anti-murine HPK1 polyclonal antibodies #5 and #6 (described supra) were used as the immunoprecipitating antibodies, and the rabbit anti-murine HPK1 antibody #2 was used for detecting HPK1 in Western blot analysis. HRP-conjugated anti-rabbit polyclonal antibody was from Amersham Biosciences. Streptavidin conjugated HRP antibody was from R&D Systems. The following antibodies were from BD Pharmingen: anti-murine CD3, CD28, PE-conjugated IL-2, rat anti-mouse biotinylated IL-2 and purified rat anti-mouse IL-2. RPMI 1640 (Cellgro, Va.) supplemented with 10% serum (Gemini Bio-products, Cat#100-602, Woodland, Calif.), β-mercaptoethanol (2-ME, 50 mM) from Gibco (CA, USA), and L-glutamine (2 mM)/penicillin (100 U/ml) and streptomycin (100 μg/ml) from Gemini Bio-Products (Woodland, Calif.) was used as a complete medium. Quillaja bark saponin and E. coli lipopolyaccharide (LPS) were from Sigma-Aldrich.

In Vitro Kinase Assay (IVK)

Splenocytes from wild type C57BL/6 were harvested, lysed with RBC lysis buffer (Sigma) and stimulated with 1 μg/ml plate bound anti-CD3 and anti-CD28 or 10 nM PGE₂ alone (Calbiochem) for 5 min. at 37° C. Whole cell lysates were subjected to immunoprecipitation with anti-HPK1 antibodies #5 and #6, then subjected to IVK as described in Kiefer et al. (1996, EMBO J. 15:7013-7025). Anti-HPK1 antibody #2 was used as a blotting antibody.

Proliferation

Naïve T cells (2×10⁵) were seeded onto a 96-well plate and incubated with 1 μg/ml anti-CD3 and anti-CD28 or 10 μg/ml ConA for three days in the presence or absence of 100 pM PGE₂ and then pulsed with 1 μCi/well ³H thymidine (MP Biomedicals, Irvine, Calif.) for 18 hours before harvesting.

Mixed Leukocyte Reaction (MLR)

MLR was performed as described in Sallusto et al. (1994, J. Exp. Med. 179:1009-1118) with minor modifications. Naïve T cells from an allogeneic SJL mouse were purified using CD62L magnetic beads (Milentyi Biotech, Inc., CA). In a 96-well round bottom plate, T cells (1×10⁵) were used as responders and incubated with HPK1^{-/-} and wild type, immature or LPS-matured bone marrow derived dendritic cells (BMDCs) as stimulators. Naïve T cells plus 10 μg/ml ConA were used as a positive control.

Enzyme Linked Immunosorbent Assay (ELISA)

Supernatants from the proliferation assay were collected for ELISA prior to addition of ³H-thymidine. The protocol used for ELISA followed the instructions from BD Pharmingen.

Intracellular Staining

Purified naïve wild type and HPK1^{-/-} splenocytes were expanded with 10 μg/ml ConA for three days with or without 10 nM PGE₂. 10⁶ cells were seeded onto a 96-well plate and cells were either left unstimulated or were stimulated with 1 μg/ml anti-CD3 and anti-CD28 for 6 hours in the presence of GolgiStop™ (BD Pharmingen). Cells were washed with buffer containing 0.1% saponin and 0.1% BSA followed by incubation with anti-IL-2-PE for 30 min. IL-2 levels were read by flow cytometry using FACSCalibur™.

Results

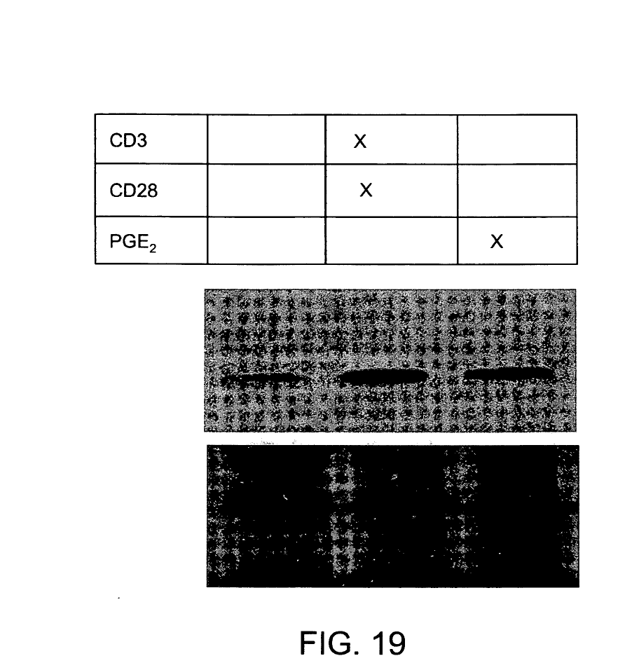

PGE₂ stimulation was demonstrated to induce HPK1 kinase activity in primary T lymphocytes (FIG. 19A, lane 3). Analysis indicated that PGE₂ stimulation induced a three-fold increase in HPK1 kinase activity compared to a four-fold induction when cells were stimulated by TCR engagement (FIG. 19A, lane 2).

To further assess the role of HPK1 in PGE₂-induced immune suppression, mice lacking HPK1 (HPK1^{-/-}) were created by disrupting exon 1 of the HPK1 gene using standard homologous recombination techniques.

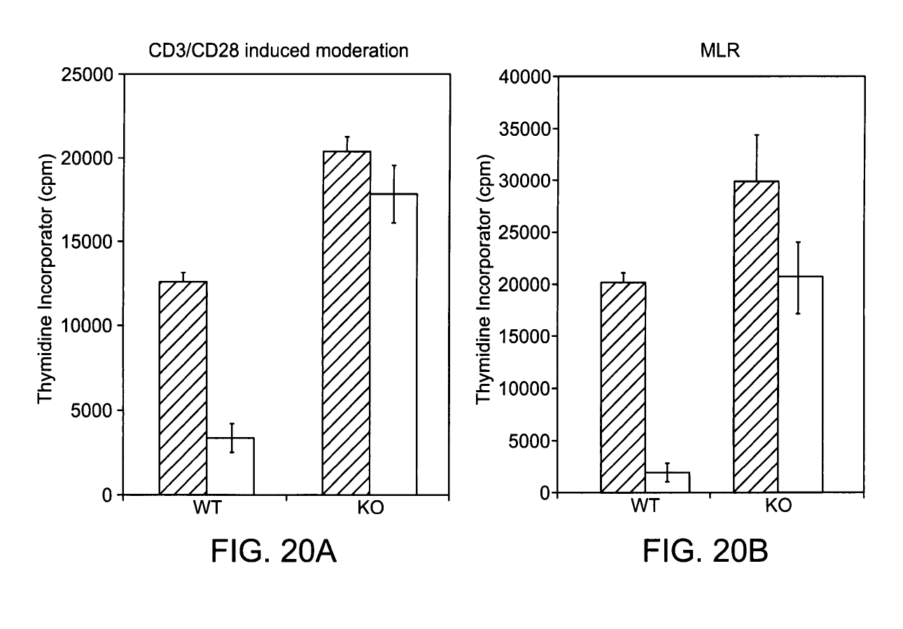

Cells from the mice were used to investigate the role of HPK1 as a negative regulator of TCR-induced gene transcription, and the role of HPK1 in PGE₂-mediated suppression of proliferation and IL-2 production. In these experiments, T cells were purified and subjected to a mixed leukocyte reaction (MLR) with wild type allogeneic BMDCs in the presence or absence of PGE₂. The level of thymidine incorporated by proliferating T cells from wild type and from HPK1^{-/-} mice indicated that the absence of HPK1 conferred T cells with a significant resistance to PGE₂-mediated inhibition (FIG. 20A). The addition of PGE₂ inhibited proliferation of the wild type T cells by ±90%, whereas HPK1^{-/-} T cells were only inhibited by ±25%. The stronger proliferative response exhibited by HPK1^{-/-} T cells may have placed these cells on a less sensitive part of the PGE₂ response curve. To assess this, the same study was repeated under conditions where the magnitude of the MLR response was similar in the two cell types by manipulating the T cell to DC ratio. It was found that the HPK1^{-/-} T cells remained resistant to PGE₂-mediated inhibition. Similar resistance to PGE₂ was observed when HPK1^{-/-} T cells were stimulated by T cell receptor activation with anti-CD3- and anti-CD28-mediated crosslinking in the presence of PGE₂ (FIG. 20B). With this form of stimulation, the addition of PGE₂ inhibited proliferation of wild type T cells by ±80%, whereas the proliferation of HPK1^{-/-} T cells was only inhibited by 18%. These data indicate that HPK1^{-/-} T cell proliferation is significantly resistant to the suppressive effects of PGE₂.

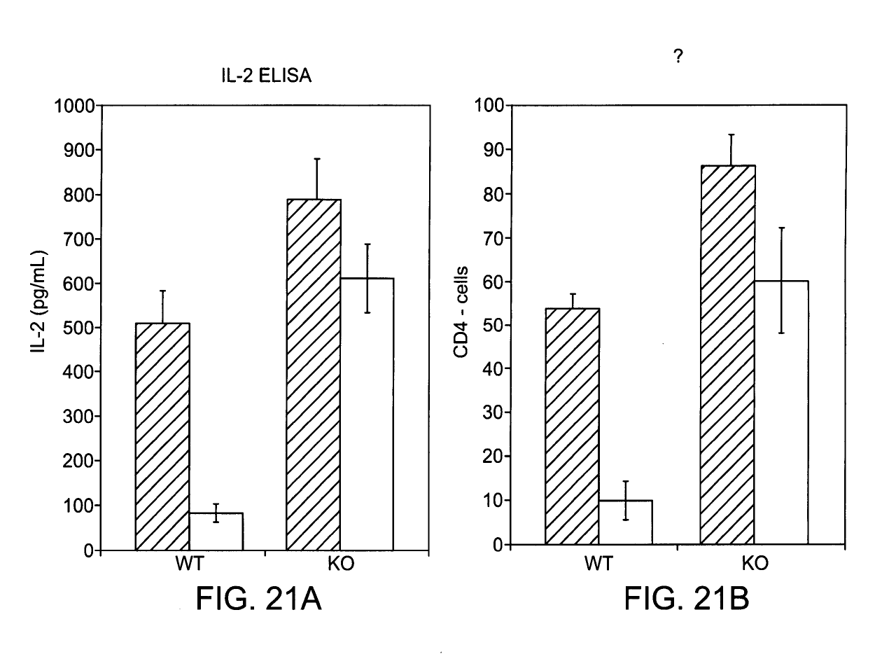

To further assess the role of HPK1 on PGE₂-mediated inhibition, supernatants from cells stimulated as described above were collected and further analyzed for IL-2 production by ELISA. The percentage of IL-2 inhibition after the addition of PGE₂ was ±30% in HPK1^{-/-} T cells compared to ±84% in wild type T cells (FIG. 21A). Intracellular levels of IL-2 (FIG. 21B) were also evaluated and the results revealed that over 45% of CD4+ HPK1^{-/-} cells still produced IL-2 after PGE₂ addition while less than 10% of wild type cells produced IL-2 after the same treatment. These findings confirm that the lack of HPK1 renders T cells resistant to PGE₂-mediated inhibition of IL-2 production.

This finding that HPK1^{-/-} T cells are significantly resistant to the suppressive effects of PGE₂ demonstrates that this hematopoietic cell-restricted serine/threonine kinase is a major component of PGE₂-induced immune suppression

These data provide further support for the role of HPK1 as a critical effector for the T cell response, e.g., to tumors producing PGE₂, and that manipulation of HPK1 expression or activity can significantly affect T cells, for example by decreasing the level of HPK1 expression or activity will make T cells more resistant to the effects of PGE₂.

### Example 5

### HPK^{-/-} Mice

The following materials and methods were used in the experiments described in Examples 6-10.

Mice, Antibodies, Media and Reagents

The establishment of HPK1 knockout mice (HPK1^{-/-}) is described supra.

Rabbit anti-murine HPK1 polyclonal antibodies numbers 5 and 6 were used as the immunoprecipitating antibodies, and the rabbit anti murine HPK1 antibody number 2 was used for detecting HPK1 in Western blot analysis. HRP-conjugated anti-rabbit polyclonal antibody was from Amersham Biosciences (Piscataway, N.J.). Streptavidin conjugated HRP antibody was from R&D systems (Minneapolis, Minn.). The following antibodies and reagents were from BD Pharmingen: anti-murine CD3, CD28, CD3-FITC, CD4-Cy5, CD8-PE, CD25-FITC, IL-2-PE, rat anti-mouse biotinylated IL-2, IFN-γ, IL-4 as well as purified rat anti-mouse IL-2, IFN-γ, IL-4, annexin V-PE and 7-amino-actinomycin D (7-AAD). RPMI 1640 (Cellgro, Herndon, Va.) supplemented with 10% serum (Gemini Bio-products, Cat#100-602), β-mercaptoethanol (2-ME, 50 uM) from Gibco (Carlsbad, Calif.), and L-glutamine (2 mM)/penicillin (100 U/ml) and streptomycin (100 ug/ml) from Gemini Bio-Products (Woodland, Calif.) was used as a complete medium. Quillaja bark saponin and concanavalin A (ConA) were from Sigma-Aldrich. Prostaglandin E 2 is from Calbiochem (San Diego, Calif.).

In Vitro Kinase Assay (IVK)

Splenocytes from wild type C57BL/6 were harvested, lysed with RBC lysis buffer (Sigma) and stimulated with 1 μg/ml plate bound anti-CD3 and 1 μg/ml soluble anti-CD28 or with 10 nM PGE₂ (Calbiochem) for 5 min. at 37° C. Whole cell lysates were subjected to immunoprecipitation with anti-HPK1 antibodies #5 and #6 and then subjected to IVK as described (1). Anti-HPK1 antibody #2 was used as a blotting antibody.

Proliferation Assay

T cells were negatively selected using the Pan T cell isolation kit (Miltenyi Biotech Inc., Auburn, Calif.). Isolated T cells were 97% pure, as assessed by anti CD3-FITC staining. 2×10⁵ Naïve T cells were seeded on a 96-well plate and incubated with various dilutions of anti-CD3 and 1 μg/ml of anti-CD28 for 72 hours in the presence or absence of 1 nM PGE₂ and then pulsed with 1 μCi/well ³H thymidine (MP Biomedicals, Irvine, Calif.) for 18 hours before harvest.

Enzyme Linked Immunosorbent Assay (ELISA)

Supernatants from the proliferation assay were collected for ELISA prior to addition of ³H thymidine. For profiling experiments, supernatants were collected as above, with variable time points. For secondary stimulation, cells were expanded with ConA for five days in the presence of 40 U/ml of IL-2 and dead cells were removed by Ficoll® gradient centrifugation (Ficoll-Paque™, Amersham Biosciences). Cells were then washed with PBS, re-seeded in fresh medium and stimulated with 2 μg/ml plate bound anti-CD3 and 1 μg/ml soluble anti-CD28 for eight hours and supernatants were collected for analysis. Protocol used for ELISA follows BD Pharmingen instructions.

Intracellular staining

RBC-lysed wild type and HPK1^{-/-} splenocytes were stimulated with or without 1 nM PGE₂ and 2 μg/ml anti-CD3 plus 1 μg/ml anti-CD28 for 48 hours in the presence of GolgiStop™ (BD Pharmingen) for the last five hours of culture. For profiling experiments, RBC-lysed splenocytes were stimulated with anti-CD3 and anti-CD28 for 24 hours, and GolgiStop was added in the last five hours. Cells were then fixed with 2% paraformaldehyde followed by permeabilization with a buffer containing 0.1% saponin and 0.1% BSA and the different conjugated antibodies for 30 min. Cytokine levels were read by flow cytometry using FACSCalibur™ and analyzed using Flowjo software.

Apoptosis

For measurement of apoptosis, HPK1^{-/-} and wild type T cells were stimulated as above, in the presence or absence of PGE2, and then stained with annexin V-PE and 7AAD.

3LL Tumor Model

Wild type Lewis Lung Carcinoma (LLC) cell line was obtained from ATCC. For subcutaneous (sc) tumors, 0.25×106 LLC cells were injected sc into the right flank of wild type or HPK1^{-/-} mice. Tumor growth over time was measured using digital calipers. Tumor volume was calculated using the formula (0.4)×(ab²), where a is the larger diameter and b is the smaller diameter. Mice were sacrificed when tumors reached 1.5 cm in diameter or 5% of the mouse size. This was used as the end point for all sc experiments due to IACUC institutional regulations. For intravenous (iv) tumor experiments, 0.5×10⁶ LLC cells were injected iv into the retro-orbital sinus. Mice were sacrificed two weeks post cell injection using carbon dioxide asphyxiation, and lung tissue was collected and fixed in 10% buffered formalin for histological analysis. Both sc and iv tumor experiments used the same protocol for the use of COX-2 inhibitor. 200 μl of 2.5 mg/kg of COX-2 inhibitor (COX-2 inhibitor II, Calbiochem) in PBS was administered to mice intraperitoneally three times per week.

Histology

Lung tissue was stained with hematoxylin and eosin (H&E), as well as anti-CD3, anti-CD4, and anti-CD8 antibodies for immunohistochemistry analysis. Tumor burden was assessed by microscopic examination of H&E stained sections that showed visible foci, and which were examine and compiled. The number of foci in the entire lung was determined using SigmaScan (Systat software, Richmond, Calif.) and the number was used as the total tumor burden.

### Example 6

### Additional Analysis of Mice Lacking HPK1

PGE₂ stimulation was found to induce HPK1 kinase activity in primary T lymphocytes (FIGS. 19A-19B). Densitometric analysis indicated that PGE₂ stimulation induced a three-fold increase in HPK1 kinase activity compared to a four-fold induction when cells are stimulated by T cell receptor (TCR) engagement.

To assess the possible role of HPK1 in PGE₂-induced immune suppression, mice lacking HPK1 (HPK1^{-/-}) were created using a homologous recombination technique as described supra.

The General Phenotype of HPK1^{-/-} Mice

Initial phenotyping of 6-8 week old HPK1^{-/-} mice revealed normal basic immune cell development and life span (Table 1). Although the life span of these mice is normal, we observed an age-related discrepancy in weight gain in mice older than 6 months, as compared to wild type mice. Concurrent with weight gain is an increase in spleen size (FIG. 18A), often leading to splenomegaly (FIG. 18B). Despite the occurrence of splenomegaly, T cell ratio in the spleen was normal, in comparison to wild type splenic T cells, as assessed by anti CD3, CD4 and CD8 staining (Table 2).

TABLE 1

| Phenotype | CD3 | CD4 | CD8 | CD3+CD4+ | CD3+CD8+ |
|---|---|---|---|---|---|
| HPK1^{+/+} | 26% | 14.80% | 8.70% | 40.17% | 33.12% |
| HPK1^{-/-} | 22% | 10.58% | 8.05% | 38.30% | 23.94% |

TABLE 2

| | Total Splenic | RBC-lysed | | | |
|---|---|---|---|---|---|
| | Cell | Splenic Cell | % CD4⁺ T | % CD8⁺ T | % Total T |
| Phenotype | Numbers | Numbers^{a} | Cells^{b} | Cells^{b} | cells (CD3⁺)^{b} |
| HPK1^{+/+} | 1.22 × 10⁸ ± 0.43 | 4.8 × 10⁷ ± 0.22 | 14.81 ± 3.41 | 8.70 ± 2.11 | 26.11 ± 2.66 |
| (n = 10; 6-12 | | | | | |
| weeks) | | | | | |
| HPK1^{-/-} | 1.42 × 10⁸ ± 0.60 | 5.5 × 10⁷ ± 0.41 | 11.25 ± 3.62 | 8.05 ± 1.06 | 22.56 ± 4.50 |
| (n = 10; 6-12 | | | | | |
| weeks) | | | | | |
| HPK1^{+/+} | 1.35 × 10⁸ ± 0.55 | 5.1 × 10⁷ ± 0.63 | 15.83 ± 3.70 | 9.33 ± 1.95 | 23.38 ± 3.78 |
| (n = 7; 6-12 | | | | | |
| months) | | | | | |
| HPK1^{-/-} (n = 7; | 2.31 × 10⁸ ± 0.77 | 9.5 × 10⁷ ± 0.81 | 17.43 ± 5.22 | 10.77 ± 21.44 | 25.86 ± 4.89 |
| 6-12 months) | | | | | |

^{a}Total numbers determined by microscopic counting.
^{b}Total percentage determined by flow cytometry.

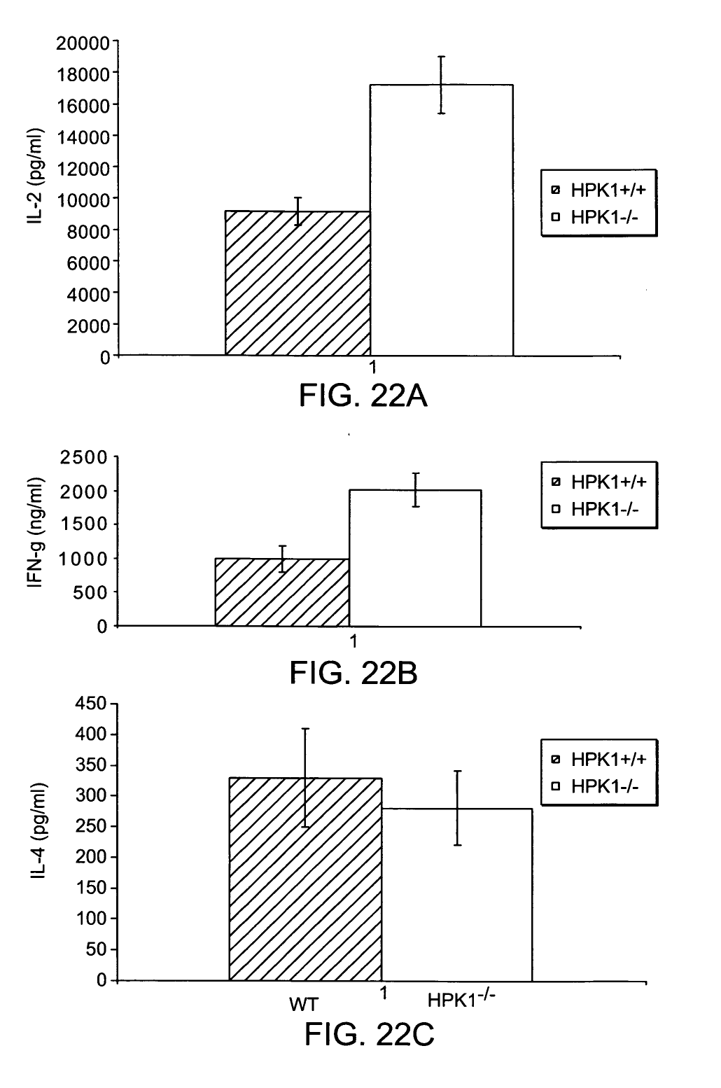

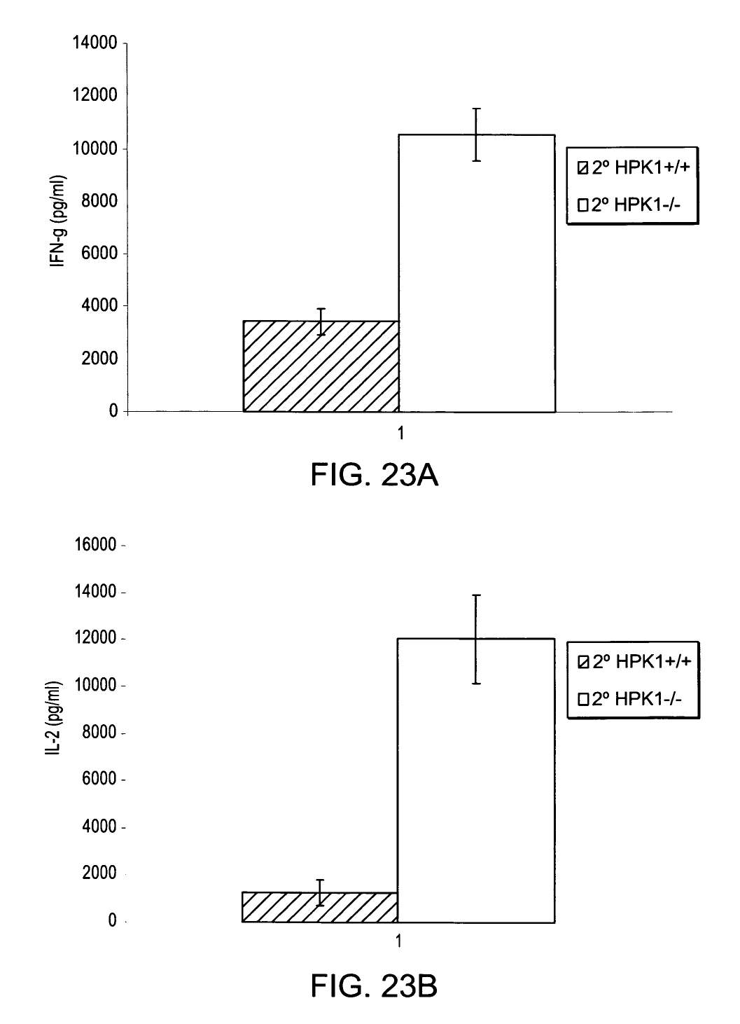

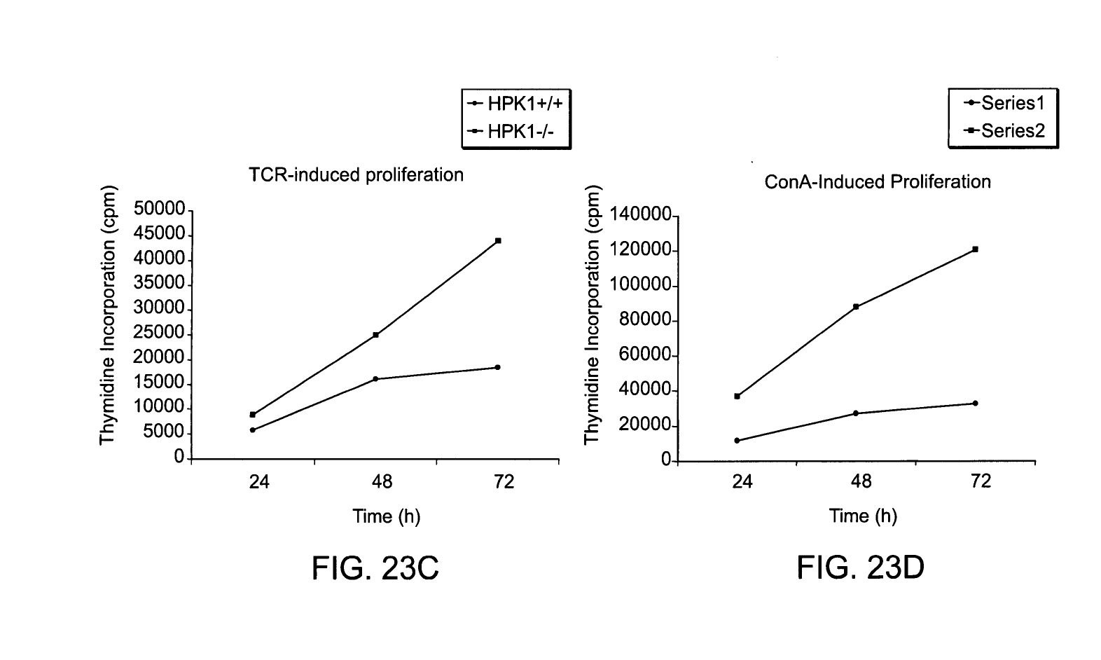

Basic cytokine analysis of IL-2, IFN-γ, and IL-4 production by T cells from HPK1^{-/-} mice revealed a weak primary skew toward a proinflammatory T helper 1 (Th1) profile (FIG. 22A-FIG. 22C), which was significantly enhanced during secondary stimulation (FIGS. 23A and 23B). HPK1 is known to be able to down regulate IL-2 gene transcription in Jurkat T cells. However, the increase in IFN-γ is a novel finding indicating a role for HPK1 in regulating pro-inflammatory cytokine production.

T cell proliferation is primarily controlled by the T cell growth factor, IL-2. An increase in IL-2 production by HPK1^{-/-} T cells indicates that these cells proliferate at a higher rate than wild type T cells. Indeed, when T cells were activated by TCR cross-linking or with concanavalin A as a positive control, thymidine incorporation by HPK1^{-/-} T cells was increased compared to that of wild type T cells (FIG. 23C and FIG. 23D). These data demonstrate that although HPK1^{-/-} mice are physically normal, T cell analysis revealed a superior Th1 skew coupled with stronger proliferative responses than in wild type C57BL/6 animals.

### Example 7

### PGE₂-Mediated Suppression of IL-2 Production in HPK^{-/-} Cells

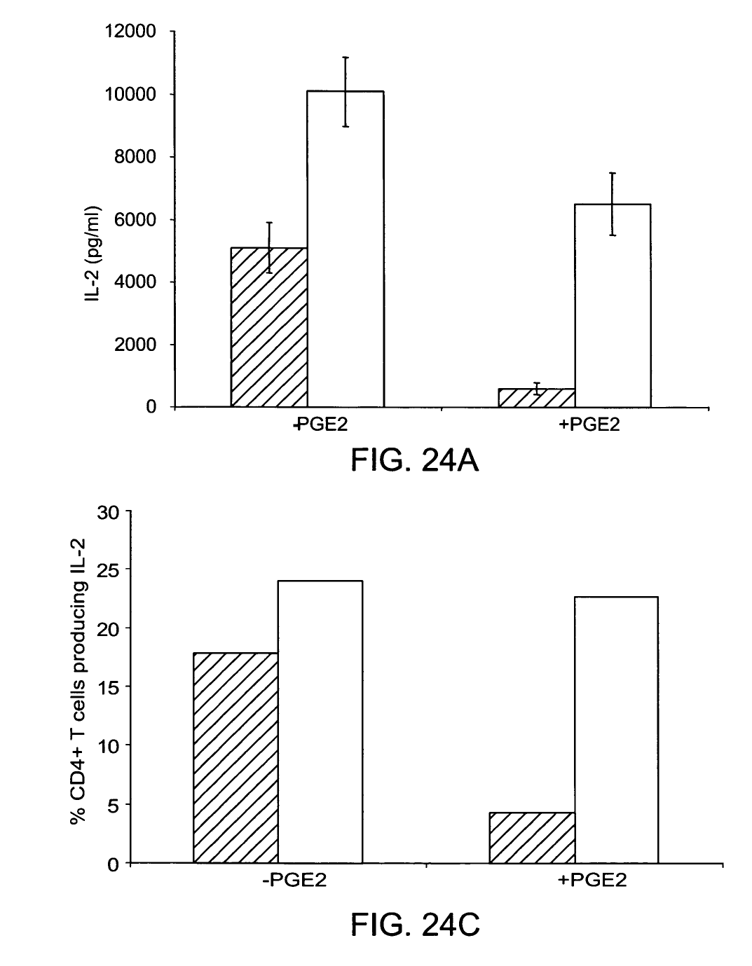

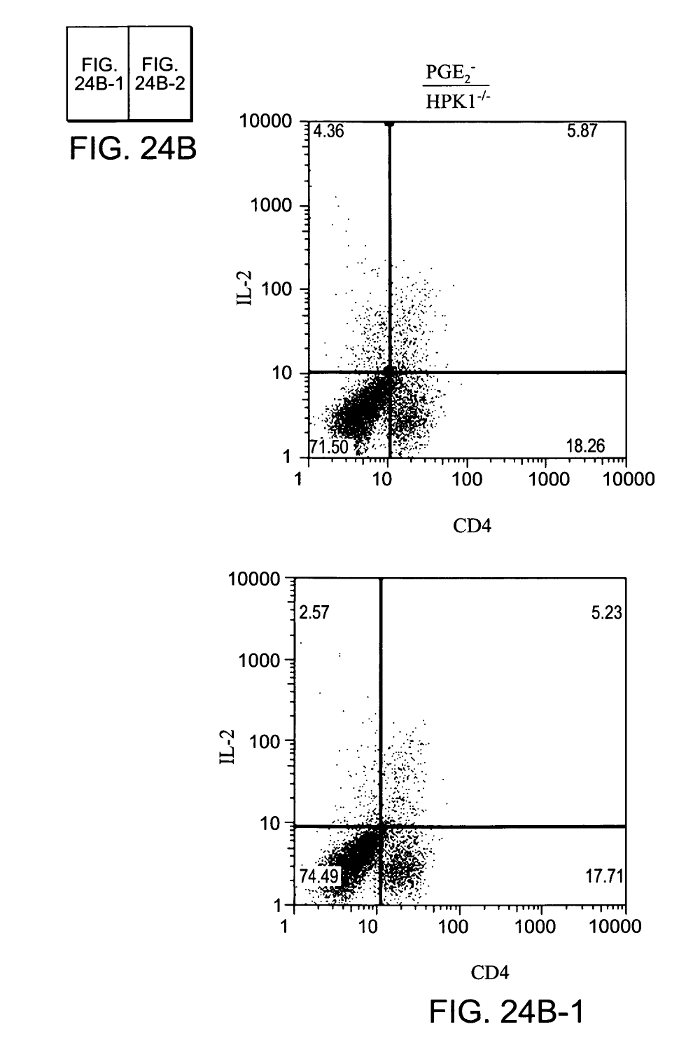

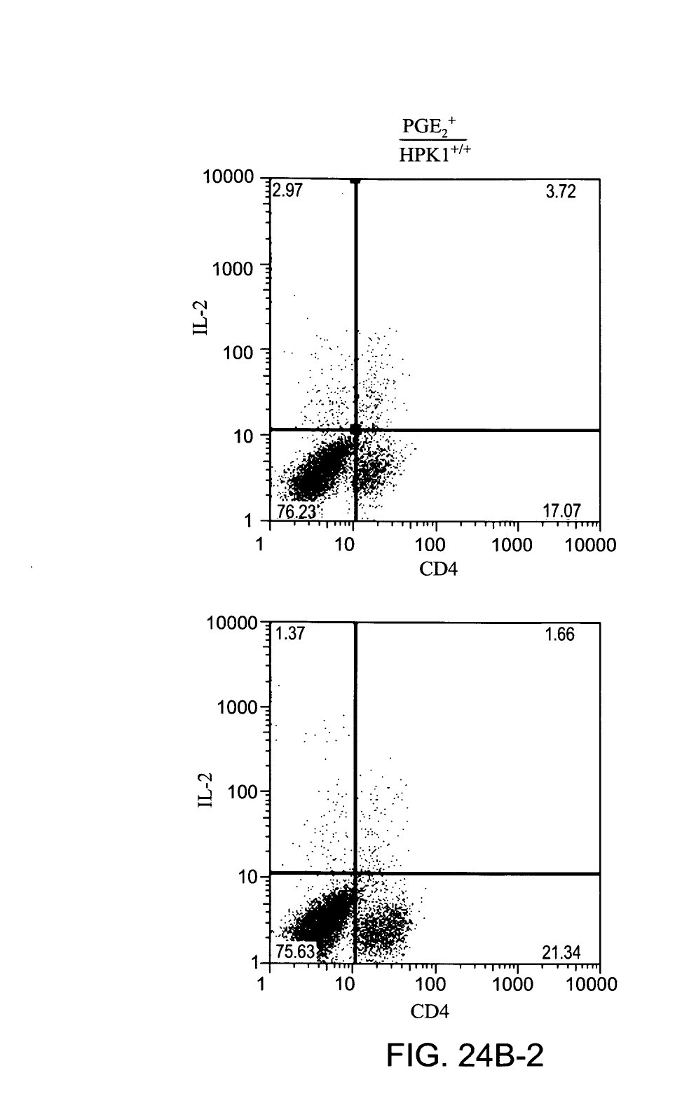

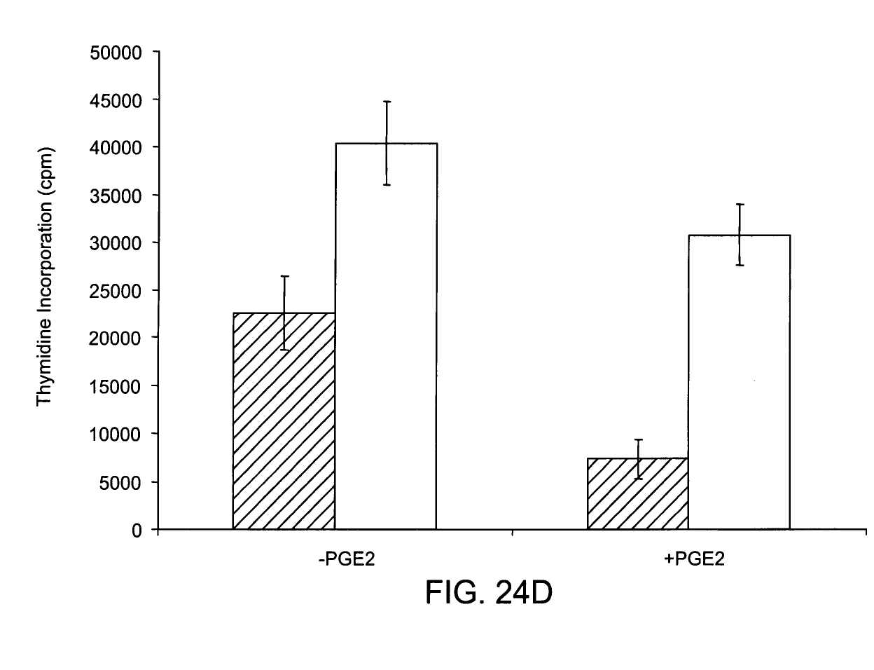

The role of HPK1 in PGE₂-mediated suppression of IL-2 production (6-9) was investigated. In these studies, purified T cells from wild type or HPK1^{-/-} mice were TCR cross-linked in the presence or absence of PGE₂, and supernatants were collected and analyzed for IL-2 production by ELISA. PGE₂ was found to inhibit IL-2 production by ±26% in HPK1^{-/-} T cells compared to ±88% in wild type cells (FIG. 24A) as assayed in supernatants from 3 day TCR stimulated cells that were collected prior to thymidine addition and assayed for IL-2 levels by ELISA. Intracellular levels of IL-2 (FIG. 24B) were also evaluated and the results revealed that over 22% of CD4+ HPK1^{-/-} T cells still produced IL-2 after co-culture with PGE₂, while fewer than 5% of wild type cells produced IL-2 under the same conditions (FIG. 24C).

These findings confirm that the lack of HPK1 renders T cells resistant (refractory) to PGE₂-mediated inhibition of IL-2 production. Thus, a compound that inhibits HPK1 expression or activity is useful for decreasing T cell response to PGE₂, and is useful for receiving PGE₂-mediated inhibition of IL-2 production.

### Example 8

### Resistance of HPK1^{-/-} T Cells to PGE₂-Mediated Inhibition of T Cell Proliferation

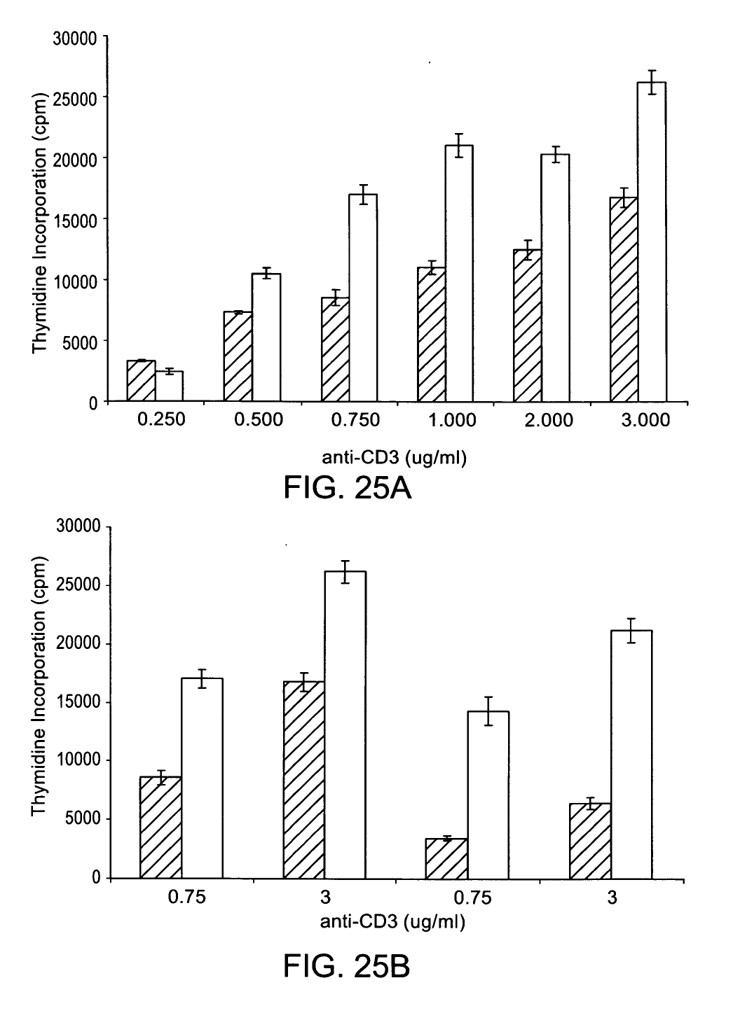

An additional immunosuppressive property of PGE₂ includes its ability to inhibit T cell proliferation. Not only does IL-2 play a critical role in regulating T cell proliferation, but, as demonstrated herein, the lack of HPK1 endogenously enhances the rate of T cell proliferation. The question of whether the addition of PGE₂ has an effect on the proliferation of T cells lacking HPK1 was examined. When T cells from wild type or HPK1 deficient mice were stimulated by TCR cross-linking, the level of thymidine incorporation indicated that the absence of HPK1 conferred T cells with a significant resistance to PGE₂-mediated inhibition (FIG. 24D). The addition of PGE₂ inhibited the proliferation of the wild type T cells by ±80%, whereas HPK1^{-/-} T cells were only inhibited by ±20%. Because it could be that the stronger proliferative response exhibited by HPK1^{-/-} T cells placed these cells on a less sensitive part of the PGE₂ response curve, the same study was repeated under conditions where the magnitude of proliferation was equivalent between wild type and HPK1^{-/-} T cells by titrating the concentration of anti-CD3 (FIG. 25A). It was found that at a concentration of 0.5 μg/ml of anti-CD3, HPK1^{-/-} T cells proliferate at a similar rate as wild type cells when stimulated with 3 μg/ml of anti-CD3. This condition was then used as a standard and the proliferation assay was repeated to include PGE₂. The results demonstrate that HPK^{-/-} T cells remained significantly resistant to PGE₂-mediated inhibition (FIG. 25B). These data demonstrate that HPK1^{-/-} T cell proliferation is significantly resistant to the suppressive effects of PGE₂. Thus, inhibition of HPK1 expression or activity is useful for increasing T cell proliferation.

### Example 9

### The Effect of PGE₂ Enhances on the Rate of Apoptosis

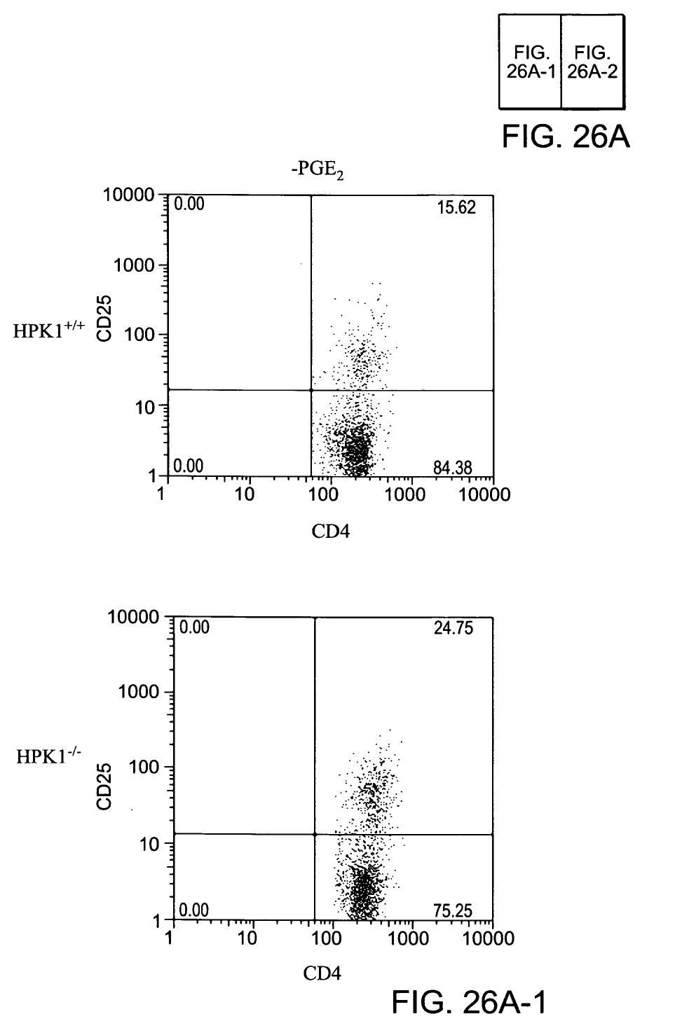

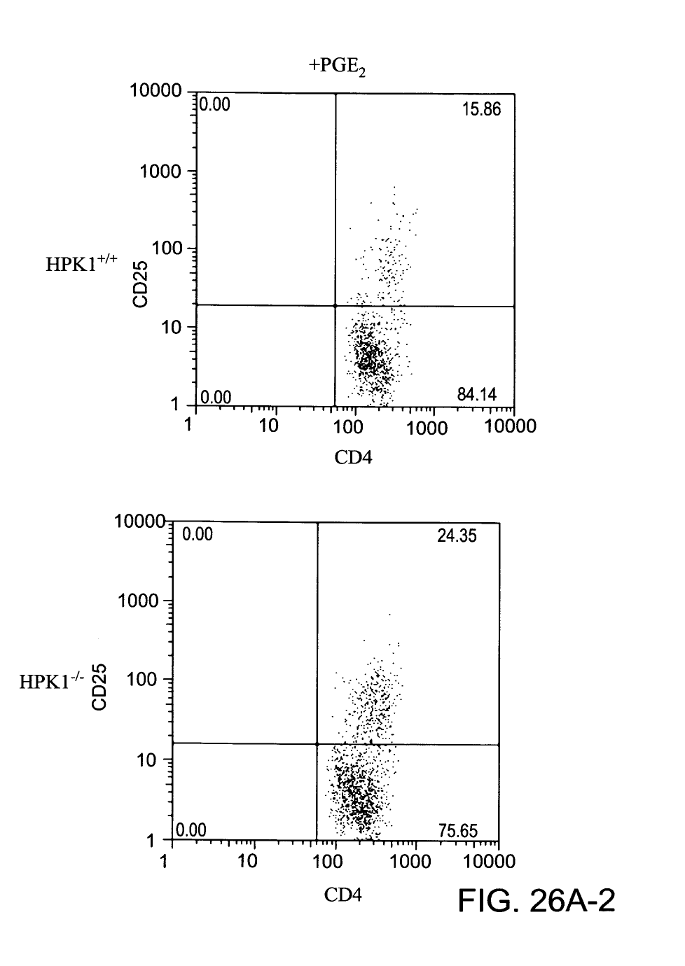

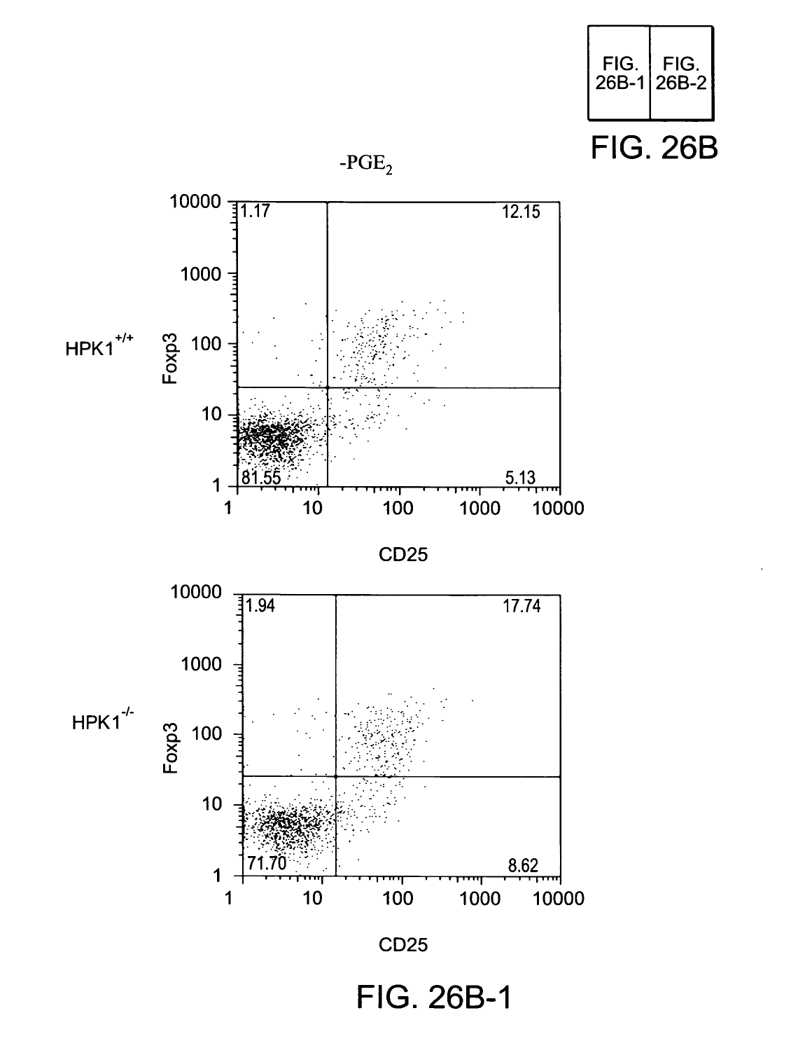

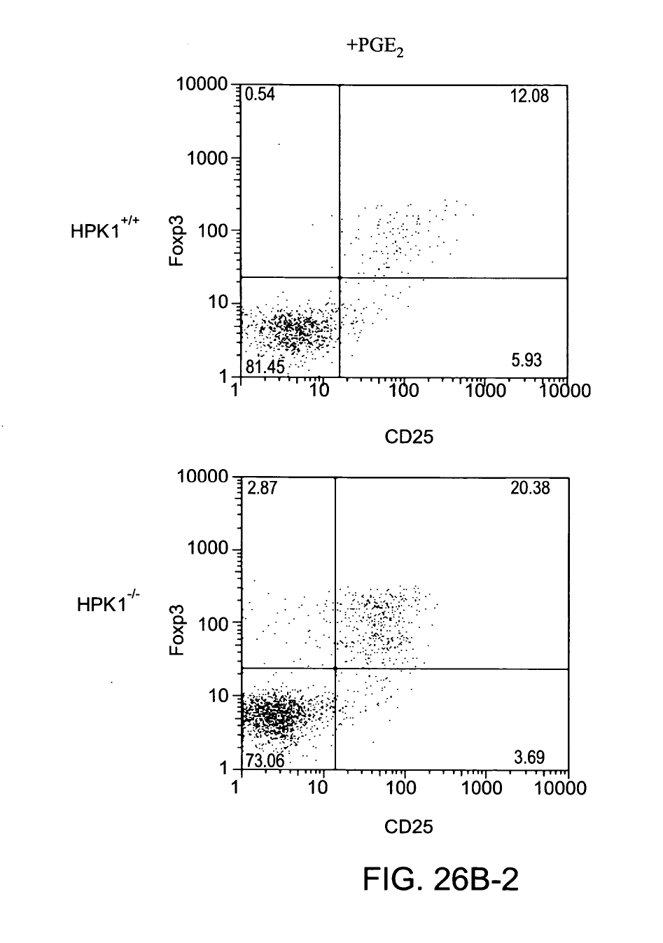

The finding that HPK1^{-/-} T cells are significantly resistant to the suppressive effects of PGE₂ implicates this hematopoietic cell-restricted serine/threonine kinase as a major component of PGE₂-induced immune suppression. However, after three days of culture with TCR cross-linking in the presence or absence of PGE₂, it was found that the percentage of CD4+ T cells expressing the IL-2 receptor, CD25, was higher in the HPK1^{-/-} culture than in control HPK1^{+/+} cells (FIG. 26A). This is due in part to the fact that HPK^{-/-} T cells produce more IL-2. However, other factors may play a role in the increased percentage of cells expressing CD25. To determine whether regulatory T cell (Treg) transcription factor, Foxp3, was a factor, the expression of CD25 in HPK^{-/-} cells was examined in this population. It has been shown that PGE₂ induces the expression of Foxp3 in both human and mouse T cells. Because of the inhibitory nature of these T cell subsets, there was a question of whether the conditions of stimulation generated fewer Tregs in the HPK1^{-/-} culture, as this might explain the enhanced functionality of these T cells.

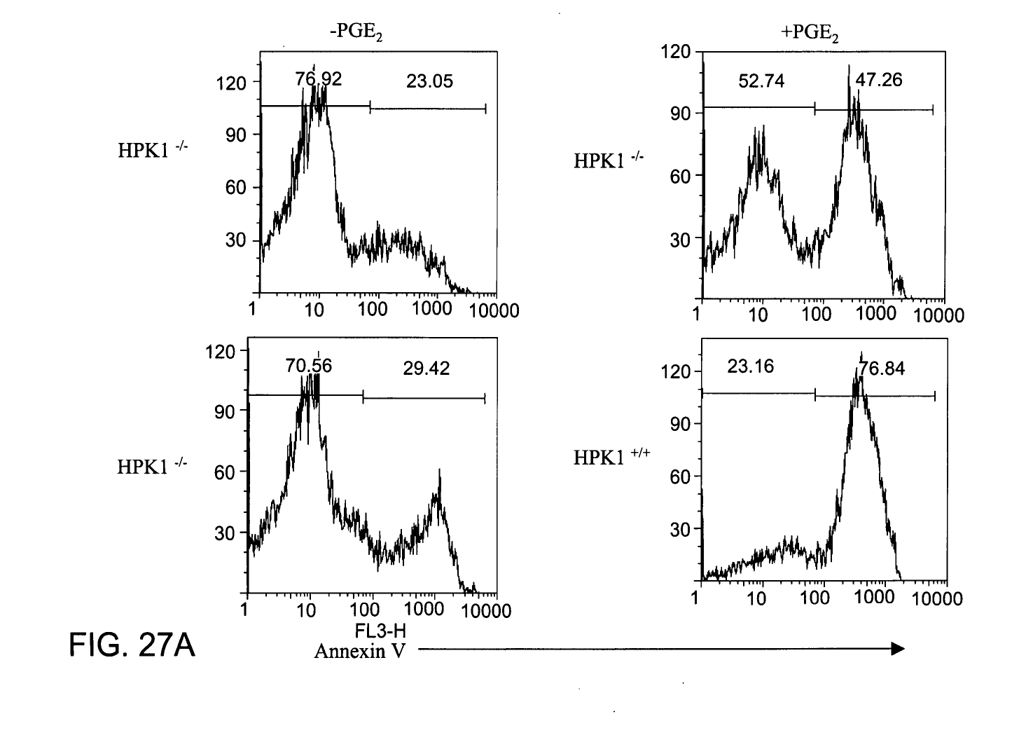

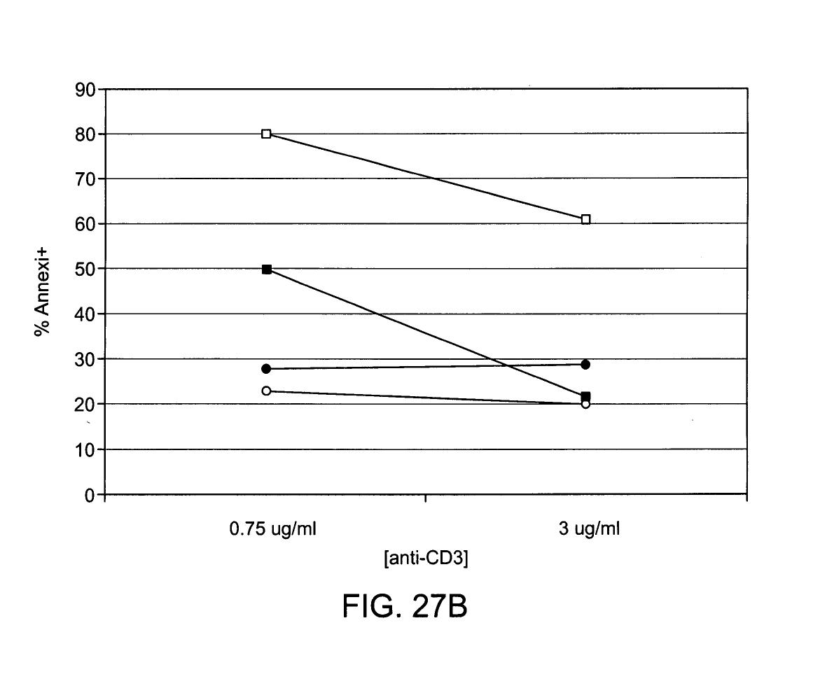

In experiments examining Foxp3 expression, PGE₂ did not induce a higher Foxp3 expression in HPK^{+/+} (wild type C57BL/6) or HPK^{-/-} cells as compared to conditions in which PGE₂ is not present (FIG. 26B). These data eliminated the involvement of Tregs as a mechanistic basis for the phenomenon of increased IL-2 expression. However, a decrease was detected in the number of cells in the side-scatter profile of wild type T cells exposed to PGE₂, indicating a decrease in cell viability. Remarkably, HPK1^{-/-} T cells showed no difference in the pattern of side-scatter with or without PGE₂. Indeed, when these cells were stained for annexin V to determine the amount of apoptosis, PGE₂ did increase the number of wild type cells undergoing apoptosis by over 75%, compared to 47% in cells not treated with PGE₂. HPK1^{-/-} T cells were significantly resistant to apoptosis after three days of TCR stimulation, since HPK1 has been shown to induce apoptosis of Jurkat T cells. The addition of PGE₂ did not significantly increase the rate of HPK1^{-/-} T cell apoptosis (FIG. 27A).

The ability of PGE₂ to enhance the apoptosis of wild type T cells, in combination with the resistance of HPK1 deficient T cells to this effect might not only explain the nature of PGE₂-induced suppression of T cell activity, but also the ability of HPK1^{-/-} T cells to withstand this inhibition. The question was examined of whether or not apoptosis is responsible for the PGE₂ resistant phenotype that HPK1^{-/-} T cells exhibit. Since HPK1^{-/-} T cells undergo less apoptosis in general, without the involvement of PGE₂, PGE₂ could not be used as a regulatory parameter for the amount of apoptosis. However, the addition of exogenous IL-2 to wild type T cells rescued their susceptibility to apoptosis, the amount of exogenous IL-2 was titrated to equate the rate of wild type and HPK1^{-/-} T cell apoptosis (FIG. 27B). The results demonstrated that the addition of IL-2 only partially rescues the proliferative phenotype of wild type T cells, and that HPK1^{-/-} T cells undergoing the same rate of apoptosis were still superior to wild type T cells in their incorporation of thymidine. These findings indicate that the involvement of HPK1 in PGE₂-induced immune suppression is partially dependent on its role as a pro-apoptotic molecule. Thus, compounds that can inhibit HPK1 are useful for decreasing apoptosis, e.g., of T cells.

### Example 10

### HPK^{-/-} T Cell Resistance to Tumor Development

To further examine the scope of HPK1^{-/-} status on T cells, a lung tumor engraftment model was used (as described in Example 5). Tumor engraftment was carried out in HPK1^{-/-} mice and in C57BL/6 (control) mice. The engrafted tumors were wild type 3LL cells (Lewis Lung cells). Tumor size was monitored using calipers to measure the dimensions of tumors, and histologic analysis of tumors was performed using H&E staining and anti-TCR, anti-CD4 or CD8 staining.

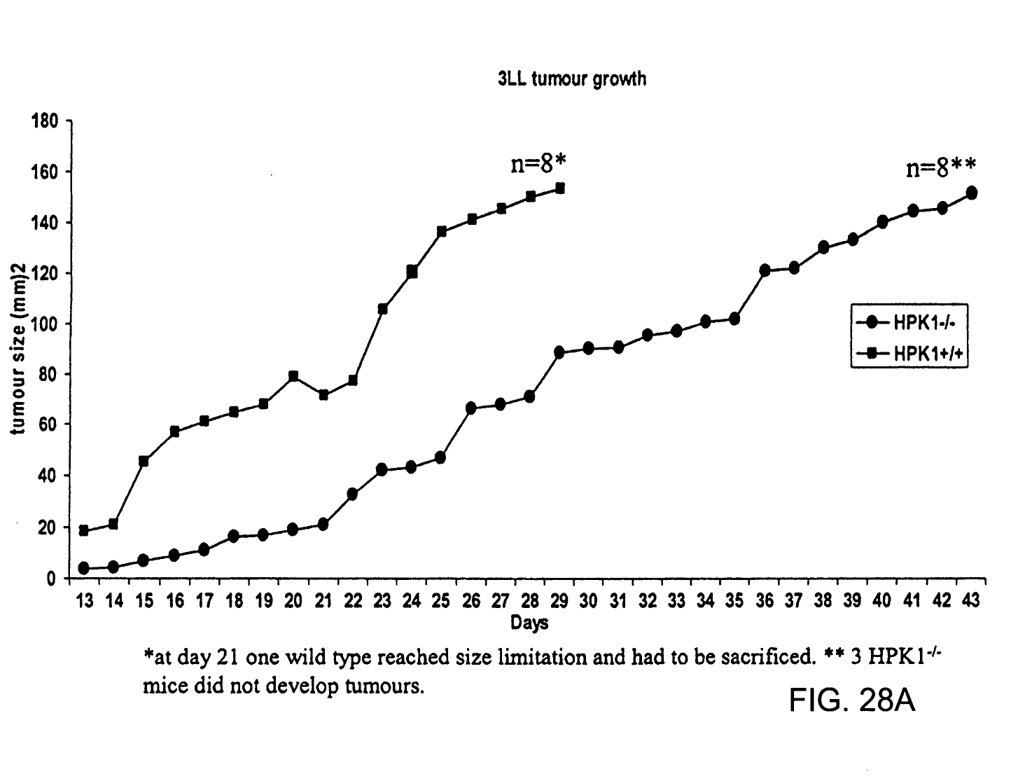

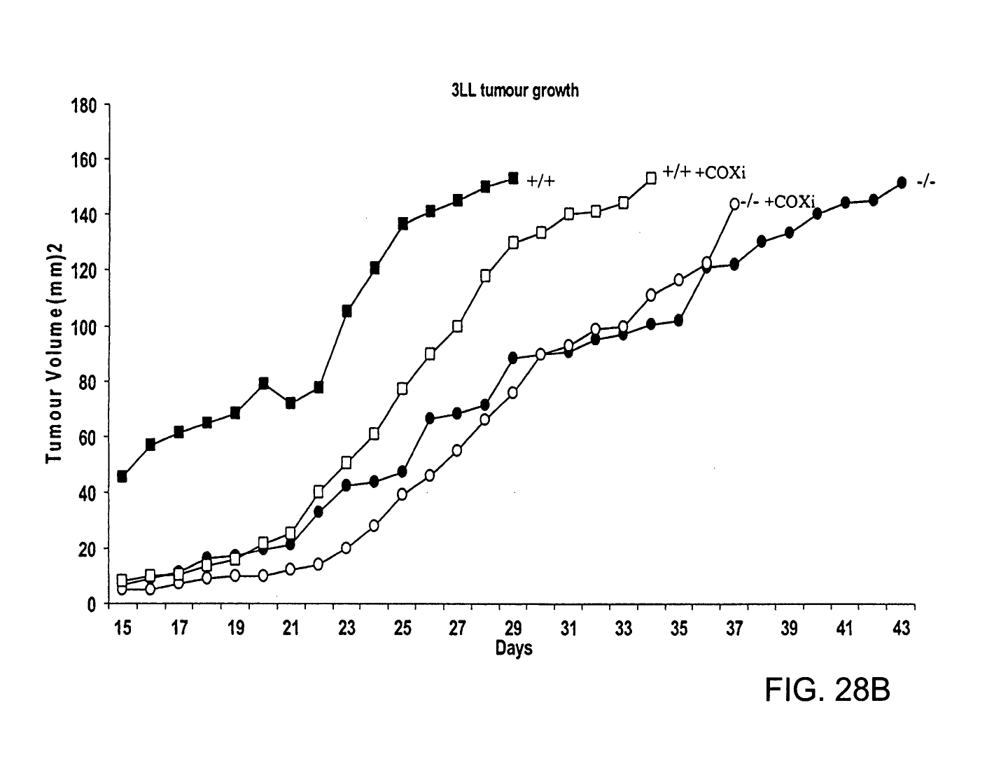

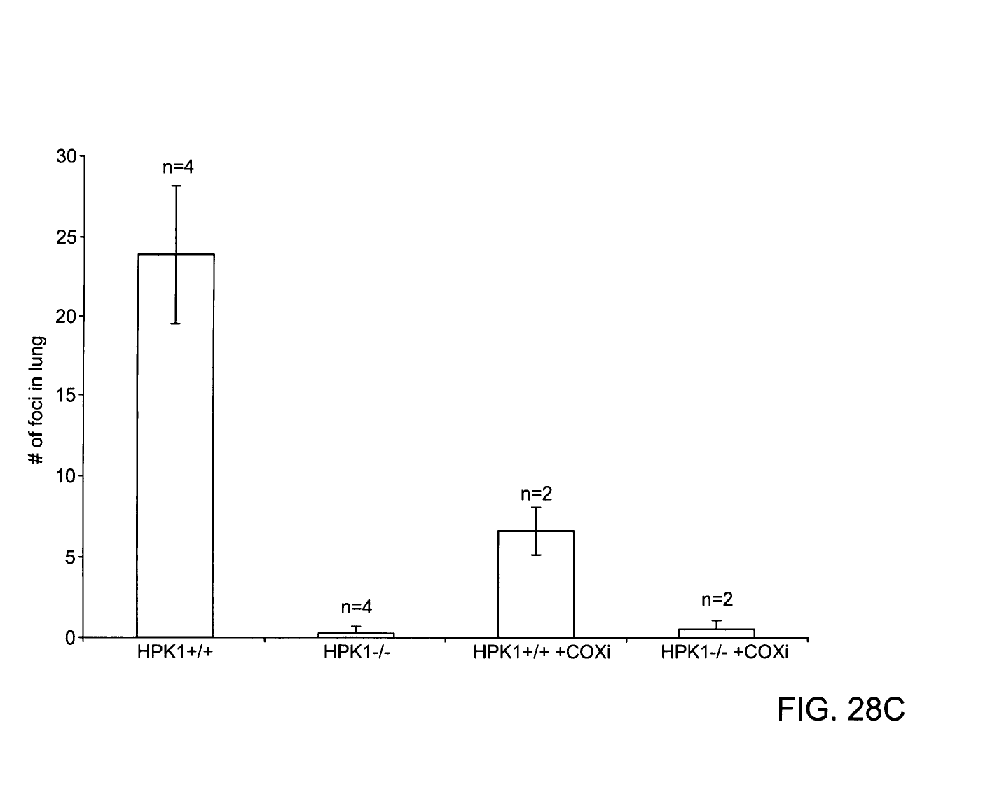

In general, it was found that tumors developed more slowly in HPK1^{-/-} mice (FIG. 28A and FIG. 28B). Also, there were generally more lymphocytic infiltrates in grafted tumors in HPK1^{-/-} mice compared to controls (FIG. 28C).

These data indicate that down regulation of HPK1 expression or activity can increase lymphocytic presence in tumors, thus providing a treatment option for treating tumors.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a candidate compound for modulating hematopoietic progenitor kinase 1 (HPK1) activity, the method comprising:
a. contacting an HPK1 or a fragment thereof comprising ser171 with a test compound; and
b. determining whether the test compound binds to the HPK1 or fragment thereof at a site that modulates protein kinase A (PKA) binding or phosphorylation of the ser171,
wherein a compound that binds to the HPK1 or fragment thereof at a site that modulates the PKA binding or phosphorylation of the ser171 is a candidate compound for modulating HPK1 activity.

2. The method of claim 1, wherein the test compound decreases PKA binding or phosphorylation of the ser171 and is a candidate compound for inhibiting HPK1 activity.

3. The method of claim 1, wherein the test compound increases PKA binding or phosphorylation of the ser171 and is a candidate compound for enhancing HPK1 activity.

4. The method of claim 1, wherein the compound inhibits HPK1 activation and enhances dendritic cell maturation or migration.

5. The method of claim 1, wherein the compound increases HPK1 activation and decreases dendritic cell maturation or migration.

6. The method of claim 1, wherein the compound is a cell permeable peptide, a pseudo substrate for HPK1, or a competitive inhibitor of ATP binding to a PKA kinase domain.

7. A method of identifying a candidate compound for enhancing dendritic cell maturation or migration, the method comprising:
a. contacting a cell with a test compound; and
b. determining whether the test compound decreases HPK1 expression or activity compared to a control cell that is not contacted with the test compound,
wherein a compound that decreases HPK1 expression or activity is a candidate compound for enhancing dendritic cell maturation or migration.

8. The method of claim 7 further comprising determining whether the test compound increases dendritic cell maturation or migration.

9. The method of claim 8, wherein dendritic cell maturation is induced by lipopolysaccharide (LPS) or a maturation stimulus.

10. The method of claim 7, wherein increased dendritic cell maturation is indicated by increased expression or increased activity of at least one of CD80 (B7.1), CD86 (B7.2), CD83, MHC class II, or CCR7.

11. A method of enhancing maturation or migration of dendritic cells, the method comprising
contacting a cell with a compound that decreases the level of HPK1 expression or HPK1 activity in a dendritic cell compared to the level of expression in a dendritic cell that is not contacted with the compound, wherein the decrease in the HPK1 expression or activity indicates at least one of increased maturation of dendritic cells or migration of dendritic cells.

12. The method of claim 11, wherein the contacted cell is a dendritic cell.

13. The method of claim 11, wherein dendritic cell maturation is induced by lipopolysaccharide (LPS).

14. The method of claim 11, wherein the compound is an siRNA.

15. The method of claim 11, wherein the expression or activity of CD80 (B7.1), CD86 (B7.2), CD83, MHC class II, or CCR7 is increased by contact with the compound.

16. The method of claim 11, wherein the dendritic cell is in a mammal.

17. The method of claim 11, wherein the dendritic cell is in a human.

18. A method of identifying a candidate compound for modulating HPK1 activity, the method comprising:
a) contacting a cell that expresses HPK1 with a test compound; and
b) determining whether the test compound modulates Cbl-b expression or activity in the contacted cell;
wherein the ability of the test compound to modulate Cbl-1 is indicative of the ability of the compound to modulate HPK1 activity.

19. The method of claim 18, further comprising determining whether the test compound can modulate HPK1 activity.

20. The method of claim 18, wherein the test compound decreases Cbl-b expression or activity and decreases HPK1 activity in the cell.

21. The method of claim 18, wherein the ubiquitin ligase activity of Cbl-b is not decreased by the test compound in the contacted cell.

22. The method of claim 18, wherein the test compound is an siRNA.

23. A method of identifying a candidate compound for modulating an immune response, the method comprising:
a. contacting a cell that expresses HPK1 with a test compound;
b. determining whether the test compound modulates Cbl-b expression or activity in the contacted cell; and
c. determining whether the test compound modulates HPK1 activity in the contacted cell,
wherein the test compound that modulates Cbl-1 and HPK1 activity is a candidate compound for modulating an immune response.

24. The method of claim 23, wherein the test compound decreases Cbl-1 expression or activity and decreases HPK1 activity in the contacted cell.

25. The method of claim 23, wherein the test compound does not decrease the ubiquitin ligase activity of Cbl-1 in the contacted cell.

26. A method of identifying a candidate compound for modulating a T cell response in an animal, the method comprising:
a. administering a test compound to an animal, the compound having the ability to decrease Cbl-b expression or activity in a cell that expresses HPK1; and
b. determining whether the animal has an increased T cell response to T cell receptor (TCR) stimulation compared to a wild type animal to which the test compound was not administered,
wherein an increased or decreased T cell response in the animal is indicative of the ability of the test compound to modulate T cell activity, and the test compound is a candidate compound for modulating HPK1 activity.

27. The method of claim 26, wherein the increased response to TCR stimulation is at least one of splenomegaly, hyperproliferation of at least one type of hematopoietic cell, resistance to PGE₂-induced immune suppression, or augmented dendritic cell function.

28. A method of identifying a compound that enhances an immune response, the method comprising:
a. administering a compound to a hematopoietic cell; and
b. assaying the hematopoietic cell for at least one indication of HPK1 inhibition,
wherein HPK1 inhibition is indicative of an enhanced immune response.

29. The method of claim 28, wherein the indication of HPK1 inhibition is splenomegaly, hyperproliferation of at least one type of hematopoietic cell, resistance to PGE₂-induced immune suppression, or augmented dendritic cell function.

30. The method of claim 28, wherein the hematopoietic cell is in an animal.

31. The method of claim 30, wherein augmented dendritic cell function is assayed by comparing dendritic cells from an animal that was administered the compound, with dendritic cells from an animal that was not administered the compound, and wherein augmented dendritic cell function is indicated by at least one of increased expression of at least one maturation marker, priming/activation of T cells, migration of cells to regional lymph nodes, secretion of IL-12, secretion of IL-6, and secretion of TNF-alpha.

32. The method of claim 31, wherein the maturation marker is CD80 (B7.1), CD86 (B7.2), CD83, MHC class I and II, CCR7, CD1a, CD1b, CD1c, CD1d, CD40, DC-LAMP, or DC-SIGN.

33. The method of claim 31, wherein the maturation marker is assayed using a Western blot, Northern blot, real time PCR, or FACS.

34. The method of claim 28, wherein HPK1 inhibition is indicated by at least one of enhanced migration of cultured dendritic cells toward a CCL-21 gradient, increased proliferation of T cells, increased stimulation of T cells, increased migration of dendritic cells in vivo, or increased Th1 cytokine production by T cells, or increased Th2 cytokine production by T cells.

35. The method of claim 31, wherein the enhanced immune response is increased T cell proliferation in the presence of an activator of TCR or ConA T cell mitogen.

36. The method of claim 32, wherein the enhanced immune response is assayed in T cells that are restimulated with an activator or TCR or ConA T cell mitogen.

37. The method of claim 28, wherein the level of PGE₂-induced immune suppression is assayed and a decrease in PGE₂-induced immune suppression indicates inhibition of HPK1.

38. A method of modulating an immune response, the method comprising contacting an immune system cell with a compound that modulates HPK1 expression or activity.

39. The method of claim 38, wherein the cell is a dendritic cell (DC).

40. The method of claim 39, wherein the compound decreases HPK1 activity and increases dendritic cell maturation relative to a dendritic cell that was not contacted with the compound.

41. The method of claim 39, wherein the compound increases HPK1 activity and decreases dendritic cell maturation is relative to a dendritic cell that was not contacted with the compound.

42. The method of claim 39, wherein the compound decreases HPK1 activity in the presence of a dendritic cell maturation factor relative to a dendritic cell that was not contacted with the compound.

43. The method of claim 41, wherein the dendritic cell maturation factor is a lipopolysaccharide (LPS).

44. The method of claim 38, wherein the immune system cell is a T cell or a B cell.

45. The method of claim 38, wherein the cell is a monocyte/macrophage, neutrophil, polymorph, natural killer cell, natural killer T cell, eosinophil, granulocytes, erythrocytes, or mast cell.

46. The method of claim 38, wherein the cell is in a human.

47. The method of claim 38, wherein the compound is an siRNA.

48. A method of identifying a subject at risk for, or having, an immune disorder or immune system cancer, the method comprising:
a) determining the level of HPK1 activity in a cell obtained from the subject; and
b) comparing the level of HPK1 activity in the cell to a non-immune disorder reference level of HPK1 or immune system cancer reference level of HPK1,
wherein a decreased level of HPK1 in the cell compared to the reference indicates that the subject is at risk for, or has, an immune disorder or an immune system cancer.

49. The method of claim 48, wherein the cell is a hematopoietic cell.

50. The method of claim 48, wherein the subject is a human.

51. The method of claim 48, wherein the immune disorder or immune system cancer is adult leukemia lymphoma (ALL), chronic myelogenous leukemia, Hodgkin's disease, Hodgkin's lymphoma, plasmacytoma, multiple sclerosis, rheumatoid arthritis, diabetes (type I), and lupus.

52. A method of identifying a subject at risk for or having a cancer, the method comprising:
a) determining the level of HPK1 activity in a cell obtained from the subject; and
b) comparing the level of HPK1 activity in the cell to a non-cancer reference level of HPK1,
wherein an increased level of HPK1 compared to the reference indicates that the subject is at risk for or has a cancer.

53. The method of claim 52, where in the cancer is lung cancer, breast cancer, prostate cancer, testicular cancer, a head or neck carcinoma, liver cancer, or bladder cancer.

54. The method of claim 52, wherein the cell is a hematopoietic cell.

55. The method of claim 52, wherein the subject is a human.

56. A method of treating a subject at risk for, or having, an immune disorder, the method comprising providing to a subject a pharmaceutically effective amount of a compound that inhibits HPK1 expression or activity, thereby decreasing the risk for having the immune disorder or treating the immune disorder.

57. The method of claim 56, wherein the compound is provided in a pharmaceutically acceptable excipient.

58. The method of claim 56, wherein the subject is a human.

59. A method of treating a subject at risk for, or having, a cancer, the method comprising providing to a subject a pharmaceutically effective amount of a compound that inhibits HPK1 expression or activity, thereby decreasing the risk for having the cancer or treating the cancer.

60. The method of claim 59, wherein the compound is provided in a pharmaceutically acceptable excipient.

61. The method of claim 59, wherein the subject is a human.

62. A method of altering at least one HPK1-mediated effect, the effect comprising increasing IL-2 production, increasing TNF secretion, increasing IFN-γ production increasing T cell proliferation, increasing B cell proliferation decreasing synthesis of an immunosuppressive cytokine, or decreasing apoptosis of T cells, decreasing tumor-induced apoptosis of hematopoetic cells, the method comprising,
a. providing a cell or organism that can express IL-2, TNF, IFN-γ, or an immunosuppressive cytokine, or providing a T cell that can proliferate, a B cell that can proliferate, or a tumor cell; and
b. contacting the cell or organism with a compound that inhibits HPK1 expression or activity in an amount and for a time sufficient to inhibit HPK1 expression or activity compared to a reference
thereby altering at least one HPK1-mediated effect, the effect comprising increasing IL-2 production, increasing TNF secretion, increasing IFN-γ production increasing T cell proliferation, increasing B cell proliferation, decreasing synthesis of an immunosuppressive cytokine, or inducing apoptosis of a tumor cell.

63. The method of claim 62, wherein the cell or organism is a non-small lung cancer cell or an organism having a non-small lung cancer.

64. A method of specifically altering PGE₂ modulation of the immune system, the method comprising contacting an immune cell with a modulator of HPK1, resulting in a change in a PGE2-modulated effect on the immune system.

65. The method of claim 64, wherein the modulator increases expression or activity of HPK1 and decreases immune activity of the immune cell.

66. The method of claim 64, wherein the modulator decreases expression or activity of HPK1 and increases immune activity of the immune cell.

67. The method of claim 64, wherein the immune cell is a T cell.

68. The method of claim 66, wherein the cell is a T cell and the increased immune activity comprises increased expression of at least one TH1 cytokine compared to a reference.

69. The method of claim 68, wherein the TH1 cytokine is Il-2 or gamma-interferon.

70. The method of claim 64, wherein PGE₂ activity is not substantially altered in non-immune system cells when the non-immune system cell is contacted with the modulator of HPK1.

71. The method of claim 64, wherein the modulator of HPK1 specifically binds HPK1.

72. A method of promoting anti-tumor immunity, the method comprising administering to a subject a compound that inhibits HPK1 expression or activity, thereby increasing anti-tumor immunity of the subject.

73. The method of claim 72, wherein the compound is a small molecule.

74. The method of claim 72, wherein the compound specifically binds to HPK1.

75. The method of claim 72, wherein the compound enhances a function selected from the group consisting of T cell effector function, natural killer cell function, and antigen presentation function.

76. The method of claim 72, wherein the enhanced function is in a dendritic cell.

77. The method of claim 37, wherein the level of PGE₂-induced immune suppression is determined by assaying IL-2 production in the presence of physiological concentration of PGE₂, and wherein sustained release of IL-2 in the presence of PGE₂ indicates HPK1 inhibition.

78. The method of claim 30, wherein splenomegaly in the animal is an indication of HPK1 inhibition.
